(19) United States
(12) Patent Application Publication
(10) Pub. No.: US 2014/0106354 A1
(43) Pub. Date: Apr. 17, 2014

(54) Method of Diagnosing Cancer

(71) Applicant: GARVAN INSTITUTE OF MEDICAL RESEARCH, Darlinghurst, New South Wales (AU)

(72) Inventors: Brian Gloss, Darlinghust (AU); Philippa O'Brien, Lilyfield (AU); Susan Clark, Darlinghurst (AU)

(73) Assignee: GARVAN INSTITUTE OF MEDICAL RESEARCH, Darlinghurst, New South Wales (AU)

(21) Appl. No.: 14/112,006

(22) PCT Filed: Apr. 18, 2012
(86) PCT No.: PCT/AU2012/000397

§ 371 Date: Dec. 27, 2013

(30) Foreign Application Priority Data
Apr. 18, 2011 (AU) 2011901456

(51) Int. Cl. C12Q 1/68

(52) U.S. Cl. CPC C12Q 1/6886
USPC 435/6.11

(57) ABSTRACT

The present invention provides methods for diagnosing cancer (such as ovarian, breast, or colon cancer) or a predisposition thereto in a subject, for monitoring the efficacy of treatment of a subject suffering from cancer, or for determining the likelihood of survival of a subject suffering from cancer, comprising detecting modified chromatin (such as DNA methylation) within a locus of the LOC134466 gene in a sample taken from the subject. The invention further provides kits for use in these methods, and methods of treating subjects based on a diagnosis performed using the methods of the invention.

This application is associated with and claims priority from Australian provisional patent application number 2011901456, filed on 18 Apr. 2011, entitled “Method of diagnosing cancer”, the subject matter of which is incorporated herein in its entirety.

### FIELD

The present invention relates to methods for diagnosing, prognosing or monitoring cancer.

### BACKGROUND

Cancer is the second leading cause of human death next to coronary disease. Worldwide, millions of people die from cancer every year. In the United States alone, cancer causes the death of well over a half-million people annually, with about 1.4 million new cases diagnosed per year. While deaths from heart disease have been declining significantly, those resulting from cancer generally are on the rise. In the early part of this century, cancer is predicted to become the leading cause of death.

Worldwide, several cancers stand out as the leading killers, including prostate cancer, breast cancer and ovarian cancer. These and virtually all other carcinomas share a common lethal feature. With very few exceptions, metastatic disease from a carcinoma is fatal. Moreover, even for those cancer patients who initially survive their primary cancers, they may suffer a recurrence or secondary cancer.

Worldwide, prostate cancer is the fourth most prevalent cancer in men. In North America and Northern Europe, it is the most common cancer in males and is the second leading cause of cancer death in men. In the United States alone, over 40,000 men die annually of this disease, second only to lung cancer. Early detection and diagnosis of prostate cancer currently relies on digital rectal examinations (DRE), prostate specific antigen (PSA) measurements, transrectal ultrasonography (TRUS), and transrectal needle biopsy (TRNB). At present, measurement of serum PSA levels in combination with DRE represents the most commonly used method used to detect and diagnose prostate cancer.

In general, a serum PSA level above 4 ng/ml is considered indicative of prostate cancer, and a serum PSA level above 10 ng/ml is considered highly suggestive of prostate cancer. However, PSA levels are also increased in benign prostatic hyperplasia, infection and chronic inflammation. Moreover, studies performed as part of the Prostate Cancer Prevention Trial demonstrated that 15% of men who had a “normal” PSA level suffered from prostate cancer (Thompson et al., N. Eng. J. Med., 350: 2239-2246, 2004). Two additional studies have also reported that PSA screening had little or no effect on mortality (Eckersberger et al., Rev. Urol., 11: 127-133, 2009).

Ovarian cancer is the fifth leading cause of cancer death in women, the leading cause of death from gynecological malignancy, and the second most commonly diagnosed gynecologic malignancy (The Merck Manual of Diagnosis and Therapy Section 18. Gynecology And Obstetrics Chapter 241. Gynecologic Neoplasms). More than 50% of women with ovarian cancer are diagnosed in the advanced stages of the disease because no cost-effective screening test for ovarian cancer exists. The five year survival rate for all stages is only 35% to 38%. If, however, diagnosis is made early in the disease, five-year survival rates can reach 90% to 98%.

Worldwide, breast cancer is the fifth most common cause of cancer death (after lung cancer, stomach cancer, liver cancer, and colon cancer). In 2005, breast cancer caused 502,000 deaths (7% of cancer deaths; almost 1% of all deaths) worldwide. Among women worldwide, breast cancer is the most common cancer and the most common cause of cancer death. In the United States, breast cancer is the third most common cause of cancer death (after lung cancer and colon cancer). Among women in the U.S., breast cancer is the most common cancer and the second most common cause of cancer death (after lung cancer). Women in the US have a 1 in 8 lifetime chance of developing invasive breast cancer and a 1 in 33 chance of breast cancer causing their death.

Breast cancer is diagnosed by the pathological (microscopic) examination of surgically removed breast tissue. A number of procedures can obtain tissue or cells prior to definitive treatment for histological or cytological examination. Such procedures include fine-needle aspiration, nipple aspirates, ductal lavage, core needle biopsy, and local surgical excision biopsy. These diagnostic steps, when coupled with radiographic imaging, are usually accurate in diagnosing a breast lesion as cancer. Occasionally, pre-surgical procedures such as fine needle aspirate may not yield enough tissue to make a diagnosis, or may miss the cancer entirely. Imaging tests are sometimes used to detect metastasis and include chest X-ray, bone scan, CT, MRI, and PET scanning. While imaging studies are useful in determining the presence of metastatic disease, they are not in and of themselves diagnostic of cancer. Only microscopic evaluation of a biopsy specimen can yield a cancer diagnosis.

As will be apparent to the skilled artisan from the foregoing description, there is a need in the art for methods for diagnosing cancer, such as ovarian cancer, breast cancer or prostate cancer.

### SUMMARY

The inventors have now determined that DNA within the LOC134466 gene is hypermethylated in cancer, e.g., in breast cancer, ovarian cancer, colon cancer (or “colorectal” cancer), head and neck cancer, lung cancer and pancreatic cancer and that LOC134466 RNA expression is reduced in cancer, e.g., breast cancer, ovarian cancer, colon cancer, head and neck cancer, lung cancer, pancreatic cancer and prostate cancer. For example, the inventors have determined that DNA within the LOC134466 gene is hypermethylated in ovarian cancer, colon cancer, and breast cancer. The inventors have shown that DNA methylation or reduced expression of LOC134466 is useful for distinguishing cancer cells from non-cancer cells. Thus, the level of DNA methylation or expression of LOC134466 is useful for distinguishing ovarian cancer, colon cancer, breast cancer, head and neck cancer, lung cancer, pancreatic cancer and/or prostate cancer cells from non-cancer cells. The level of DNA methylation or expression of LOC134466 is particularly useful for distinguishing ovarian cancer, colon cancer and/or breast cancer cells from non-cancer cells. The inventors have additionally shown that DNA methylation or reduced expression of LOC134466 RNA is useful for detecting various sub-types of cancer, e.g., epithelial ovarian cancer and sub-types thereof, such as clear cell carcinoma and serous carcinoma. The inventors have observed these changes in various distinct forms of cancer, meaning that the invention is generally applicable to cancer as well as to specific types of cancer. The inventors have also found that methods for detecting changes in, for example, methylation levels are robust, in so far as they permit detection in various samples treated in various manners, e.g., frozen, fixed and/or paraffin embedded. In addition, the inventors have found that such methods can be performed reliably in bodily fluid samples such as blood, plasma, or other bodily fluid samples to detect changes and/or status of, for example, methylation levels of any of the genes disclosed herein.

Based on the findings of the inventors, the present disclosure provides various methods for diagnosing, prognosing or monitoring cancer generally, as well as specific types of cancer, e.g., ovarian cancer, breast cancer, colon cancer or prostate cancer. In one particular example, the present disclosure provides various methods for diagnosing, prognosing or monitoring ovarian cancer.

For example, the present disclosure provides a method for diagnosing a cancer or a predisposition thereto in a subject, the method comprising detecting in a sample from the subject:

(i) modified chromatin relative to a non-cancerous sample the modified chromatin being positioned within a locus containing a LOC134466 gene and/or

(ii) modified expression relative to a non-cancerous sample of the LOC134466 RNA,

wherein the modified chromatin and/or the modified expression is diagnostic of a cancer or a predisposition thereto in the subject.

When used herein the phrase “locus containing a LOC134466 gene” includes reference to a locus which contains a LOC134466 gene and includes CpG #25 (chr5:150306098-150306387(hg18)) and extends upstream to CpG#23 (chr5:150264579-150264828 (hg18)) and downstream to CpG#43 (chr5:150264579-150264828(hg18)).

The present disclosure additionally or alternatively provides a method for monitoring the efficacy of treatment of a subject receiving treatment for a cancer, the method comprising identifying and/or detecting in a sample from the subject:

(i) unmodified chromatin or less-modified chromatin relative to a non-cancerous sample the unmodified chromatin or less-modified chromatin being positioned within a locus containing a LOC134466 gene; and/or

(ii) unmodified expression relative to a non-cancerous sample of the LOC134466 RNA,

wherein the unmodified chromatin or less-modified chromatin and/or the unmodified expression indicates that the treatment is effective.

The present disclosure additionally or alternatively provides a method for monitoring the efficacy of treatment of a subject receiving treatment for a cancer, the method comprising identifying and/or detecting in a sample from the subject:

(i) modified chromatin relative to a non-cancerous sample the modified chromatin being positioned within a locus containing a LOC134466 gene; and/or

(ii) modified expression relative to a non-cancerous sample of the LOC134466 RNA,

wherein the modified chromatin and/or the modified expression indicates that the treatment is not effective.

The present disclosure additionally or alternatively provides a method for determining the likelihood of survival of a subject suffering from a cancer, the method comprising identifying and/or detecting in a sample from the subject

(i) modified chromatin relative to a non-cancerous sample the modified chromatin being positioned within a locus containing a LOC134466 gene; and/or

(ii) modified expression relative to a non-cancerous sample of the LOC134466 RNA,

wherein the modified chromatin and/or the modified expression indicates that the subject is less likely to survive. The presence of an increased level of modified chromatin and/or expression in a sample taken from a subject relative to the level of modified chromatin and/or expression in a non-cancerous sample indicates a reduced likelihood of survival for the patient. Thus, the presence of modified chromatin and/or modified expression in a sample taken from the subject indicates that the subject is less likely to survive. Conversely, the presence of a reduced level of modified chromatin and/or expression in a sample taken from a subject relative to the level of modified chromatin and/or expression in a non-cancerous sample indicates an increased likelihood of survival for the patient.

In one example, the modified chromatin is within a LOC134466 gene. In another example, the modified chromatin is within a SGNE1 gene. In accordance with this example, any example included herein comprising detecting modified chromatin within a LOC134466 locus or gene or detecting modified expression of LOC134466 shall be taken to apply mutatis mutandis to detecting modified chromatin with a SGNE1 gene or modified SGNE1 expression.

In one example, a method of the disclosure is a multiplex or multi-analyte method. Thus, the method may comprise detecting modified chromatin and/or expression of other genes and/or pseudogenes in addition to the locus comprising LOC134466. For example, the method may additionally comprise detecting modified chromatin nucleic acid positioned within a locus containing a (or within a) gene or pseudogene and/or modified expression of the gene or pseudogene in the sample. In one example, the gene or pseudogene is selected from the group consisting of ARMCX1, ICAM4, IL18, PEG3, PYCARD, SGNE1, ZNF177, HSPA2, KLF4, LTBP2, PAPLN, PARVA, PTGER, SCIN1, SPOCK2, TLE4, ZNF542, BMP6, CST6, SOCS1 and combinations thereof. Preferred genes are selected from the group consisting of ARMCX1, ICAM4, IL18, PEG3, PYCARD, SGNE1, ZNF177 and combinations thereof. For example, preferred genes may be selected from ARMCX1, ICAM4, PEG3, PYCARD and SGNE1. In one example, the gene is PYCARD. In another preferred example, the gene is SGNE1.

The inventors have also identified panels of markers useful for diagnosing/prognosing cancer in a subject.

In one example, the method of the present disclosure comprises additionally detecting modified chromatin positioned within ARMCX1, ICAM4, IL18, PEG3, PYCARD, SGNE1 and ZNF177 and/or modified expression of ARMCX1, ICAM4, IL18, PEG3, PYCARD, SGNE1 and ZNF177 in the sample. In another example, the method of the present disclosure comprises additionally detecting modified chromatin positioned within ARMCX1, ICAM4, PEG3, PYCARD and SGNE1 and/or modified expression of ARMCX1, ICAM4, PEG3, PYCARD and SGNE1 in the sample. In another example, the method of the present disclosure comprises additionally detecting modified chromatin positioned within SGNE1, or within ARMCX1, or within ICAM4, or within PEG3, or within PYCARD, or within any combination of two or more of these genes, and/or modified expression of SGNE1, or ARMCX1, or ICAM4, or PEG3, or PYCARD, or any combination of two or more of these genes.

In one example, the method of the present disclosure comprises detecting modified chromatin positioned within ARMCX1, ICAM4, LOC134466, PEG3, PYCARD and SGNE1 and/or modified expression of ARMCX1, ICAM4, LOC134466, PEG3, PYCARD and SGNE1 in the sample.

In one example, the modified expression is associated with the modified chromatin or the unmodified expression is associated with (or caused by) the unmodified chromatin or less-modified chromatin.

The present disclosure encompasses diagnosis, prognosis or monitoring of any cancer. Exemplary cancers are ovarian cancer, breast cancer, colon cancer, prostate cancer, head and neck cancer, lung cancer and pancreatic cancer. Particular examples are ovarian cancer, breast cancer and/or colon cancer.

In one example, the cancer is ovarian cancer and/or breast cancer.

In one example, the ovarian cancer is clear cell carcinoma or serous carcinoma.

In one example, the breast cancer is invasive breast cancer or ductal carcinoma in situ.

In one exemplified form of the present disclosure, the modified chromatin or unmodified chromatin or less-modified chromatin is detected by performing a process comprising detecting the level of methylation of nucleic acid in the sample from the subject relative to a non-cancerous sample. In one example, the nucleic acid is DNA.

For example, the level of methylation may be detected by performing a process comprising:

(i) detecting the level of methylation of a nucleic acid within the pseudogene or gene in the sample derived from the subject;
(ii) detecting the level of methylation of the nucleic acid within the pseudogene or gene in the control sample; and
(iii) comparing the level of methylation at (i) and (ii).

For example, the level of methylation is detected by one or more of the following:

(i) performing methylation-sensitive endonuclease digestion of DNA;
(ii) treating nucleic acid from the sample with an amount of a compound that selectively mutates non-methylated cytosine residues in nucleic acid under conditions sufficient to induce mutagenesis thereof and produce a mutant nucleic acid and amplifying the mutant nucleic acid using at least one primer that selectively hybridizes to the mutant nucleic acid and/or amplifying the methylated nucleic acid using at least one primer that selectively hybridizes to the methylated nucleic acid;
(iii) treating nucleic acid from the sample with an amount of a compound that selectively mutates non-methylated cytosine residues in nucleic acid under conditions sufficient to induce mutagenesis thereof and produce a mutant nucleic acid, hybridizing a nucleic acid probe or primer capable of specifically hybridizing to the mutant nucleic acid or the methylated nucleic acid and detecting the hybridized probe or primer; and
(iv) treating nucleic acid from the sample with an amount of a compound that selectively mutates non-methylated cytosine residues in nucleic acid under conditions sufficient to induce mutagenesis thereof and produce a mutant nucleic acid and determining the nucleotide sequence of the mutant nucleic acid;

In one example, the level of methylation is detected using a headloop PCR-based method.

In one example, the compound that selectively mutates non-methylated cytosine residues is a sodium salt of bisulphite.

Exemplary nucleic acids in which methylation is detected comprise any nucleic sequence as disclosed herein. For example, nucleic acids in which methylation is detected may comprise a sequence set forth in any one or more of SEQ ID NOs: 1, 3, 4, 6, 8 and 12. For example, nucleic acid in which methylation is detected may comprise a sequence set forth in SEQ ID NO: 1 or 3. In addition, methylation may be detected in any sequence within of any of the sequences disclosed herein. For example, methylation may be detected in a sequence within any one or more of SEQ ID NOs: 1, 3, 4, 6, 8 and 12. The specific length of the sequence within any of the sequences disclosed herein may vary. In one example, the sequence within any of the sequences disclosed herein comprises one or more CpG islands (i.e., the sequence within any of the sequences disclosed herein may comprise one or more CpG dinucleotides). Methylation may be detected at, within or near a promoter region and/or a transcription start site of any of the genes or pseudogenes disclosed herein. In one example, methylation can be detected in a sequence comprising nucleic acids 103 to 120 of SEQ ID NO: 3. In another example, methylation may be detected in a sequence comprising nucleic acids 160 to 185 of SEQ ID NO: 3.

In one example, the level of expression of a nucleic acid is detected by performing a process comprising:

(i) detecting the level of expression of the pseudogene or gene in the sample from the subject; and

(ii) detecting the level of expression of the pseudogene or gene in the non-cancerous sample.

For example, the level of expression of the nucleic acid is detected by performing a process comprising hybridizing a probe or primer capable of specifically hybridizing to a transcript of the pseudogene or gene to the nucleic acid in a sample and detecting the level of hybridization by a detection means, wherein the level of hybridization of the probe or primer is indicative of the level of expression of the pseudogene or gene.

In one example, the level of expression is detected by performing a process comprising contacting the sample with an antibody or antigen binding fragment thereof capable of specifically binding to a polypeptide encoded by the pseudogene or gene for a time and under conditions for a complex to form and detecting the level of the complex by a detection means, wherein the level of the complex is indicative of the level of expression of the pseudogene or gene.

In one example, the sample comprises tissue and/or a body fluid suspected of comprising a cancer cell. For example, the sample may comprise tissue or cells from a breast, an ovary, a colon or a prostate. Exemplary body fluids are selected from the group consisting of whole blood, a fraction of blood (such as blood plasma, serum, or another fraction of blood), urine, saliva, breast milk, pleural fluid, sweat, tears and mixtures thereof. Preferably, the sample is blood or a derivative thereof, such as blood plasma or serum.

In one example, the sample is a tumor sample. In one example, the sample was frozen following collection. In one example, the sample was fixed, e.g., in formalin following collection. In one example, the sample is embedded in a solid or semi-solid substrate, e.g., paraffin.

In one example, the non-cancerous sample selected from the group consisting of:

(i) a sample comprising a non-cancerous cell;

(ii) a sample from a normal tissue;

(iii) a sample from a healthy tissue;

(iv) an extract of any one of (i) to (iii);

(v) a data set comprising measurements of modified chromatin and/or expression for a healthy individual or a population of healthy individuals;

(vi) a data set comprising measurements of modified chromatin and/or expression for a normal individual or a population of normal individuals; and

(vii) a data set comprising measurements of the modified chromatin and/or expression from the subject being tested wherein the measurements are determined in a matched sample having normal cells.

In one example, a method of the present disclosure additionally comprises providing the result of the method, e.g., in paper or computer readable form.

The present disclosure additionally provides a method of treating or preventing cancer comprising performing a method described herein according to any example to diagnose cancer or a predisposition thereto and performing or recommending therapeutic or prophylactic treatment for cancer.

### BRIEF DESCRIPTION OF THE DRAWINGS

FIG. 1 comprises two graphical representations showing methylation profiles of gene candidate lists relative to random gene selections. Panel A shows MeDIP-chip signals from candidate genes identified from A2780 cells. Panel B shows MeDIP-chip signals from candidate genes identified from CaOV3 genes. Averaged MeDIP-Chip signal (y axes) of gene candidate lists over the promoter (2 kb upstream) of the TSS (x-axes) in EOC cell lines A2780, CaOV3 and OSE. Methylation distributions of 1000 random selections of genes were plotted (grey shading) for comparison.

FIG. 2A is a series of diagrammatic and graphical representations. Panel (a) shows CpG methylation at 7 candidate gene promoters (as indicated) in cell lines A2780 and CaOV3. Average promoter methylation (percentage and greyscale) was calculated from all informative CpG units excluding overlapping peaks and CpG dinucleotides within primer sequences. Transcriptional start sites (TSS) are shown as arrows, * indicates non-canonical CpG island.

FIG. 2B includes two graphical representations showing the correlation of average DNA methylation measured by Sequenom (x-axis) and MAT analysis for gene promoters in A2780 (top panel) and CaOV3 (bottom Panel), Pearsons correlation coefficent (r) is calculated and line of best fit is plotted (dotted line).

FIG. 3A is a diagrammatic representation showing the average promoter methylation, sorted by average methylation in all samples, of the sixteen genes with evidence of methylation in the profiled cell lines as determined by Sequenom analysis in a panel of primary OSE, immortalized OSE (HOSE) and EOC cell lines.

FIG. 3B is a graphical representation showing relative mRNA levels normalized to 18s RNA of 8 methylated genes (y axis) measured against average promoter methylation (x-axis) for immortalized OSE and EOC cell lines. A line of best fit is plotted and Pearson's correlation coefficient (r) and p value are included.

FIG. 4A is a diagrammatic representation showing hierarchical clustering of average promoter methylation for 15 candidate genes in a panel of 20 serous EOC. Eight genes (enclosed by box) exhibit evidence of methylation in multiple tumor samples.

FIG. 4B is a graphical representation showing receiver operating characteristic (ROC) curves, indicating area under the curve (AUC) and significance (Mann-Whitney U test p value) for eight genes displaying methylation in ovarian tumors. and logistic regression model of six genes identified as best fitted to the data by ANOVA (black broken line).

FIGS. 5A and 5B show results of clonal bisulphite sequencing of DNA surrounding the TSS of LOC134466 from 5 tumours, 4 OSE, 4 cancer cell lines (A2780, CaOV3, OV90, UWB1.289) and one immortalised OSE (HOSE 17.1) along with controls (CpGenomoe & Roche). The transcription start site (TSS) is indicated by the arrow. Black circles indicate methylated sites and white circles indicate non-methylated sites.

FIG. 5C is a graphical representation showing expression of LOC134466 (relative to 18s) in cell lines compared with average methylation of CpG units at the TSS of the gene.

FIG. 5D is a diagrammatic representation showing hierarchical clustering of Sequenom methylation levels at CpG units flanking the TSS of LOC134466 for 69 Type II EOC tumors and 14 OSE.

FIG. 5E is a graphical representation showing averaged methylation for the region surrounding the TSS of LOC134466 in tumor samples versus OSE.

FIG. 5F is a graphical representation showing a ROC, which demonstrates low false-positive rate for cancer discrimination at high methylation levels in regions surrounding the TSS of LOC143366.

FIG. 6A is a graphical representation showing the location, relative to the TSS of LOC143366 (arrow), of primers (indicated) and CpGs (circles) interrogated by the headloop and probe (indicated) (B) Output of headloop suppression PCR by agarose gel and dissociation curve.

FIG. 6B is a graphical representation showing amplification on unmethylated template is suppressed by the headloop but can be visualized by its dissociation characteristics.

FIG. 6C is a series of graphical representations showing an exemplary dissociation curve of methylated sample and unmethylated sample with peak dissociation shown along with Tm cutoff for methylation used to in the method of determining headloop positivity (table).

FIG. 7 is a graphical representation showing the percentage of samples positive for headloop suppression PCR assay designed to interrogate CpGs at the TSS of LOC143366 for 15 OSE and 100 archival FFPE Type II EOC samples.

FIG. 8A is a graphical representation showing the proportion of matched primary breast tumor and lymph node metastasis samples positive for methylation of LOC134466.

FIG. 8B is a graphical representation showing relative methylation for primary tumours compared to the lymph node metastasis for the same patients.

FIG. 9 is a graphical representation showing relative methylation levels in samples derived from nine matched normal, DCIS and carcinoma samples.

FIG. 10 is a diagrammatic representation showing the level of methylation in LOC134466 in cell lines from breast normal epithelium and tumours as measured by both Sequenom MassARRAY and headloop suppression PCR. The region assayed using head-loop PCR is highlighted in grey.

FIGS. 11A-D are a series of graphical representations showing the level of methylation in LOC134466 in fresh frozen ovarian cancer samples (A), normal ovarian surface epithelium (B), immortalized ovarian surface epithelium (HOSE) ovarian cancers and clear cell ovarian cancer samples (C) and paraffin embedded ovarian cancer samples (D) as measured by both Sequenom MassARRAY.

FIGS. 12A and B are a series of graphical representations showing results of quantitative Sequenom analysis of methylation (bars) in cell lines A2780 and CaOV3 (A) and EOC cell lines and immortalised OSE (HOSE) (B) for CpG islands surrounding LOC134466. Genes are sorted relative to location in the genome.

FIG. 12C shows methylation patterns of CpG#23, CpG#25 and CpG#43 in the cell lines A2780 and CaOV3 relative to ovarian surface epithelium (OSE) cells as measured by MeDIP-chip.

FIG. 13A shows Receiver Operating Characteristic (ROC) curves for each of six genes LOC134466, SGNE1, ARMCX1, ICAM4, PEG3 and PYCARD, as well as a two gene panel (LOC134466 and SGNE1), whose methylation status is used to distinguish EOC from OSE.

FIG. 13B illustrates the distribution of methylation status of the six gene panel in EOC vs OEC. The left hand y axis indicates the number of samples with n genes of the six gene panel methylated. The right hand y axis indicates the cumulative percentage of samples containing n methylated genes of the six gene panel.

FIG. 13C shows the calculated sensitivity and specificity for each of the genes or combinations studied.

FIG. 14 shows the results of two experiments determining LOC134466 methylation status (one comparing plasma from 26 patients with ovarian cancer to plasma from 11 patients with non-ovarian cancers (Nov); the other comparing plasma from 12 healthy control subjects, 10 patients with ovarian cancer and 10 patients with non-ovarian cancers (Oct)).

FIG. 15 shows the proportion of plasma samples from healthy patients or cancer patients positive for LOC134466 or SGNE1 methylation, comparing plasma from 12 healthy control subjects, 10 patients with ovarian cancer and 10 patients with non-ovarian cancers.

FIG. 16A shows the methylation status of both LOC134466 and SGNE1 in various cell lines (1, dark shading=methylated; 0, light shading=unmethylated).

FIG. 16B shows the proportion of samples from various cancer cell lines containing methylated LOC134466.

FIG. 17 shows LOC134466 expression as determined by TaqMan® qRTPCR relative to DNA methylation as determined by MSH-PCR in various cell lines.

FIG. 18 shows LOC134466 methylation status in colon cancer samples compared to normal tissue (lowest sloping line).

### DETAILED DESCRIPTION

### Key to Sequence Listing

SEQ ID NO 1: nucleotide sequence of Homo sapiens zinc finger protein 300 pseudogene
SEQ ID NO 2: amino acid sequence of Homo sapiens KRAB domain-containing protein LOC134466
SEQ ID NO 3: nucleotide sequence of a region of LOC134466 for methylation analysis
SEQ ID NO 4: nucleotide sequence of Homo sapiens paternally expressed 3 (PEG3) (transcript variant 1)
SEQ ID NO 5: amino acid sequence of Homo sapiens paternally expressed 3 (PEG3) (transcript variant 1)
SEQ ID NO 6: nucleotide sequence of Homo sapiens armadillo repeat containing, X-linked 1 (ARMXC 1)
SEQ ID NO 7: amino acid sequence of Homo sapiens armadillo repeat containing, X-linked 1 (ARMXC 1)
SEQ ID NO 8: nucleotide sequence of Homo sapiens intracellular adhesion molecule 4 (ICAM4) (transcript variant 1)
SEQ ID NO 9: amino acid sequence of Homo sapiens intracellular adhesion molecule 4 (ICAM4) (transcript variant 1)
SEQ ID NO 10: nucleotide sequence of Homo sapiens interleukin 18 (IL-18)
SEQ ID NO 11: amino acid of Homo sapiens interleukin 18 (IL-18)
SEQ ID NO 12: nucleotide sequence of Homos sapiens PYD and CARD domain containing (PYCARD) (transcript variant 1)
SEQ ID NO 13: amino acid sequence of Homos sapiens PYD and CARD domain containing (PYCARD) (transcript variant 1)
SEQ ID NO 14: nucleotide sequence of Homo sapiens secretogranin V (7B2 protein/SGNE1 protein) (transcript variant 1)
SEQ ID NO 15: amino acid sequence of Homo sapiens secretogranin V (7B2 protein/SGNE1 protein) (transcript variant 1)
SEQ ID NO 16: nucleotide sequence of Homo sapiens zinc finger protein 177 (ZNF177) (transcript variant 1)
SEQ ID NO 17: amino acid sequence of Homo sapiens zinc finger protein 177 (ZNF177) (transcript variant 1)
SEQ ID NO 18: nucleotide sequence of Homo sapiens heat shock protein 2 (HSPA2)
SEQ ID NO 19: amino acid sequence of Homo sapiens heat shock protein 2 (HSPA2)
SEQ ID NO 20: nucleotide sequence of Homo sapiens kruppel-like factor 4 (KLF4)
SEQ ID NO 21: amino acid sequence of Homo sapiens kruppel-like factor 4 (KLF4)
SEQ ID NO 22: nucleotide sequence of Homo sapiens latent transforming growth factor beta binding protein 2 (LTBP2)
SEQ ID NO 23: amino acid sequence of Homo sapiens latent transforming growth factor beta binding protein 2 (LTBP2)
SEQ ID NO 24: nucleotide sequence of Homo sapiens papilin, proteoglycan-like sulphated glycoprotein (PAPLN)
SEQ ID NO 25: amino acid sequence of Homo sapiens papilin, proteoglycan-like sulphated glycoprotein (PAPLN)
SEQ ID NO 26: nucleotide sequence of Homo sapiens parvin alpha (PARVA)
SEQ ID NO 27: amino acid sequence of Homo sapiens parvin alpha (PARVA)
SEQ ID NO 28: nucleotide sequence of Homo sapiens prostaglandin E receptor 3 (subtype 3) (PTGER3 isoform 5)
SEQ ID NO 29: amino acid sequence of Homo sapiens prostaglandin E receptor 3 (subtype 3) (PTGER3 isoform 5)
SEQ ID NO 30: nucleotide sequence of Homo sapiens scinderin (SCIN) (transcript variant 1)
SEQ ID NO 31: amino acid sequence of Homo sapiens scinderin (SCIN) (transcript variant 1)
SEQ ID NO 32: nucleotide sequence of Homo sapiens sparc/osteonectin cwcv, and kazal-like domains (SPOCK2) (transcript variant 1)
SEQ ID NO 33: amino acid sequence of Homo sapiens sparc/osteonectin cwcv, and kazal-like domains (SPOCK2) (transcript variant 1)
SEQ ID NO 34: nucleotide sequence of Homo sapiens transducin-like enhancer of split 4 (E(sp1)) homolog (TLE4)
SEQ ID NO 35: amino acid sequence of Homo sapiens transducin-like enhancer of split 4 (E(sp1)) homolog (TLE4)
SEQ ID NO 36: RNA sequence of Homo sapiens zinc finger protein 542 (ZNF542) (transcript variant 1)
SEQ ID NO 37: ORF sequence of Homo sapiens zinc finger protein 542 (ZNF542)
SEQ ID NO 38: nucleotide sequence of Homo sapiens bone morphogenetic protein 6 (BMP6)
SEQ ID NO 39: amino acid sequence of Homo sapiens bone morphogenetic protein 6 (BMP6)
SEQ ID NO 40: nucleotide sequence of Homo sapiens cystatin E/M (CST6)
SEQ ID NO 41: amino acid sequence of Homo sapiens cystatin E/M (CST6)
SEQ ID NO 42: nucleotide sequence of Homo sapiens suppressor of cytokine signaling 1 (SOCS1)
SEQ ID NO 43: amino acid sequence of Homo sapiens suppressor of cytokine signaling 1 (SOCS1)
SEQ ID NO 44: BSP-T7 forward primer ARMCX1
SEQ ID NO 45: BSP-T7 reverse primer ARMCX1
SEQ ID NO 46: BSP-T7 forward primer BMP6
SEQ ID NO 47: BSP-T7 reverse primer BMP6
SEQ ID NO 48: BSP-T7 forward primer CST6
SEQ ID NO 49: BSP-T7 reverse primer CST6
SEQ ID NO 50: BSP-T7 forward primer HSPA2
SEQ ID NO 51: BSP-T7 reverse primer HSPA2
SEQ ID NO 52: BSP-T7 forward primer ICAM4
SEQ ID NO 53: BSP-T7 reverse primer ICAM4
SEQ ID NO 54: BSP-T7 forward primer IL18
SEQ ID NO 55: BSP-T7 reverse primer IL18
SEQ ID NO 56: BSP-T7 forward primer KLF4
SEQ ID NO 57: BSP-T7 reverse primer KLF4
SEQ ID NO 58: BSP-T7 forward primer LOC134466 RC
SEQ ID NO 59: BSP-T7 reverse primer LOC134466 RC
SEQ ID NO 60: BSP-T7 forward primer LTBP2 RC
SEQ ID NO 61: BSP-T7 reverse primer LTBP2 RC
SEQ ID NO 62: BSP-T7 forward primer PAPLN
SEQ ID NO 63: BSP-T7 reverse primer PAPLN
SEQ ID NO 64: BSP-T7 forward primer PARVA
SEQ ID NO 65: BSP-T7 reverse primer PARVA
SEQ ID NO 66: BSP-T7 forward primer PEG3 long
SEQ ID NO 67: BSP-T7 reverse primer PEG3 long
SEQ ID NO 68: BSP-T7 forward primer PEG3 short
SEQ ID NO 69: BSP-T7 reverse primer PEG3 short
SEQ ID NO 70: BSP-T7 forward primer PTGER3
SEQ ID NO 71: BSP-T7 reverse primer PTGER3
SEQ ID NO 72: BSP-T7 forward primer PYCARD
SEQ ID NO 73: BSP-T7 reverse primer PYCARD
SEQ ID NO 74: BSP-T7 forward primer SCIN
SEQ ID NO 75: BSP-T7 reverse primer SCIN
SEQ ID NO 76: BSP-T7 forward primer SGNE1
SEQ ID NO 77: BSP-T7 reverse primer SGNE1
SEQ ID NO 78: BSP-T7 forward primer SOCS3
SEQ ID NO 79: BSP-T7 reverse primer SOCS3
SEQ ID NO 80: BSP-T7 forward primer SPOCK2 RC
SEQ ID NO 81: BSP-T7 reverse primer SPOCK2 RC
SEQ ID NO 82: BSP-T7 forward primer TLE4
SEQ ID NO 83: BSP-T7 reverse primer TLE4
SEQ ID NO 84: BSP-T7 forward primer ZNF177
SEQ ID NO 85: BSP-T7 reverse primer ZNF177
SEQ ID NO 86: BSP-T7 forward primer ZNF542
SEQ ID NO 87: BSP-T7 reverse primer ZNF542
SEQ ID NO 88: SYBR forward primer ARMCX1
SEQ ID NO 89: SYBR reverse primer ARMCX1
SEQ ID NO 90: SYBR forward primer HSPA2
SEQ ID NO 91: SYBR reverse primer HSPA2
SEQ ID NO 92: SYBR forward primer ICAM4
SEQ ID NO 93: SYBR reverse primer ICAM4
SEQ ID NO 94: SYBR forward primer IL18
SEQ ID NO 95: SYBR reverse primer IL18
SEQ ID NO 96: SYBR forward primer LOC13446
SEQ ID NO 97: SYBR reverse primer LOC13446
SEQ ID NO 98: SYBR forward primer PEG3
SEQ ID NO 99: SYBR reverse primer PEG3
SEQ ID NO 100: SYBR forward primer PYCARD
SEQ ID NO 101: SYBR reverse primer PYCARD
SEQ ID NO 102: SYBR forward primer SCIN
SEQ ID NO 103: SYBR reverse primer SCIN
SEQ ID NO 104: SYBR forward primer TLE4
SEQ ID NO 105: SYBR reverse primer TLE4
SEQ ID NO 106: SYBR forward primer ZNF177
SEQ ID NO 107: SYBR reverse primer ZNF177
SEQ ID NO 108: ARMCXHL F 1 primer
SEQ ID NO 109: ARMCX_HL_R1 primer
SEQ ID NO 110: ARMCX_HL_R1HL primer
SEQ ID NO 111: ARMCX_HLcomPrb primer
SEQ ID NO 112: ICAM4_HL_F1 primer
SEQ ID NO 113: ICAM4_HL_F1HL primer
SEQ ID NO 114: ICAM4_HL_R1 primer
SEQ ID NO 115: ICAM4_HL_R1HL primer
SEQ ID NO 116: ICAM4_HLcomPrb primer
SEQ ID NO 117: LOC_ts_R4HL primer
SEQ ID NO 118: LOC_ts_F4 primer
SEQ ID NO 119: LOC_ts_R4 primer
SEQ ID NO 120: LOC_HLcomPrb primer
SEQ ID NO 121: PEG3_HL_F 1 primer
SEQ ID NO 122: PEG3_HL_R1 primer
SEQ ID NO 123: PEG3_HL_R1HL primer
SEQ ID NO 124: PEG3_HLcomPrb primer
SEQ ID NO 125: PYCARD_HL_μl primer
SEQ ID NO 126: PYCARD_HL_R1 primer
SEQ ID NO 127: PYCARD_HL_R1HL primer
SEQ ID NO 128: PYCARD_HLcomPrb primer
SEQ ID NO 129: SGNE1_HL_F1 primer
SEQ ID NO 130: SGNE1_HL_R1 primer
SEQ ID NO 131: SGNE1_HL_R1HL primer
SEQ ID NO 132: SGNE1_HLcomPrb primer

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and DI; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Müller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

The present invention shall be taken to encompass any markers collectively or individually as provided in any list herein. By “collectively” is meant that the invention encompasses any number or combination of the recited proteins or markers or groups of proteins or markers, and that, notwithstanding that such numbers or combinations of proteins or markers or groups of proteins or markers may not be specifically listed herein the accompanying claims may define such combinations or sub-combinations separately and divisibly from any other combination of proteins or markers or groups of proteins or markers. By “individually” is meant that the invention encompasses the recited proteins or markers or groups of proteins or markers separately, and that, notwithstanding that individual proteins or markers or groups of proteins or markers may not be separately listed herein the accompanying claims may define such protein or marker or groups of proteins or markers separately and divisibly from each other.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SELECTED DEFINITIONS

As used herein, the term “pseudogene” will be understood to mean a sequence in the genome of a subject that is a copy of a genomic gene. The pseudogene may be transcribed and/or translated or transcribe a non-coding RNA. This term also encompasses intervening intronic sequences (if present) in addition to regulatory regions that control the expression of the pseudogene (if present).

As used herein, the term “gene” means nucleic acid in the genome of a subject capable of being expressed to produce a mRNA and/or protein in addition to intervening intronic sequences and in addition to regulatory regions that control the expression of the gene, e.g., a promoter or fragment thereof.

As used herein, discussion of a nucleic acid “within” a pseudogene or a gene, will be understood to encompass a nucleic acid within a protein and/or mRNA coding region, or an intronic region or a regulatory region or other non-coding region (upstream or downstream of a mRNA or protein coding region). In one example, a nucleic acid within a LOC134466 gene that is methylated is within the regulatory region (e.g., a promoter or enhancer), optionally including a transcriptional start site.

The term “within a locus containing a pseudogene [or gene]” will be understood to mean within a region of a genome containing the pseudogene [or gene], for example a region of the genome containing pseudogene [or gene] and about 100 Mb or 50 Mb or 20 Mb or 10 Mb upstream and/or downstream of the pseudogene [or gene]. In the case of LOC134466, a region containing the pseudogene can comprise the region spanning from TNIP1 to DCN4, or from GPX3 to MST150 or from GPX3 to ZNF300 or from GPX3 to LOC134466 or from LOC134466 to ZNF300.

As used herein, the term “diagnosis”, and variants thereof, such as, but not limited to “diagnose” or “diagnosing” shall include, but not be limited to, a primary diagnosis of a clinical state or any primary diagnosis of a clinical state. A diagnostic assay described herein is also useful for assessing the remission of a subject, or monitoring disease recurrence, or tumor recurrence, such as following surgery, radiation therapy, adjuvant therapy or chemotherapy, or determining the appearance of metastases of a primary tumor. All such uses of the assays described herein are encompassed by the present disclosure.

As used herein, the term “cancer” shall be taken to include a disease that is characterized by uncontrolled growth of cells within a subject. The term “cancer” shall not be limited to cancer of a specific tissue or cell type. Those skilled in the art will be aware that as a cancer progresses, metastases occur in organs and tissues outside the site of the primary cancer. For example, in the case of many cancers, metastases commonly appear in a tissue selected from the group consisting of lymph nodes, lung, breast, liver, kidney and/or bone. Accordingly, the term “cancer” as used herein shall be taken to include a metastasis of a cancer in addition to a primary tumor. Exemplary cancers include breast cancer, ovarian cancer, colon cancer, head and neck cancer, lung cancer, pancreatic cancer and/or prostate cancer. In a preferred embodiment, the cancer is selected from the group consisting of a colon cancer, a prostate cancer, a breast cancer, an ovarian cancer or a pancreatic cancer. For example, the cancer may be a colon cancer, a prostate cancer or a breast cancer. For example, the cancer may be an ovarian cancer, a breast cancer, or a colon cancer. For example, the cancer may be a colon cancer. Alternatively, the cancer may be a prostate cancer. Alternatively, the cancer may be a breast cancer. Alternatively, the cancer may be an ovarian cancer.

As used herein, the term “chromatin” shall be taken to mean nucleic acid in the context of the genome, including (but not limited to) in the context of a complex of nucleic acid (e.g., genomic DNA) and protein (e.g., one or more histones) such as a nucleosome. As will be understood by the skilled artisan, nucleic acid and protein e.g., histones, are generally packaged to fowl nucleosomes that form in the interphase nucleus of a cell. The term “chromatin” also encompasses “naked” DNA (i.e., not associated with a protein) as is observed at transcription start sites and/or promoters of transcriptionally active genes and/or pseudogenes. It will be apparent from the disclosure herein that the state of chromatin can be determined for nucleic acid bound to protein, e.g., histone or in its naked form, e.g., methylated DNA.

As used herein, the term “modified chromatin” shall be taken to mean a change in the relative amount of euchromatin and heterochromatin in a biological sample (e.g., a cell or a cell extract) from a subject detected on the basis of any means including reduced expression of a gene, hypermethylation or deacetylation of nucleic acid and/or histone. In one example, the term “modified chromatin” means an increased or reduced level of methylation of DNA. In the present context, modified chromatin is generally determined with reference to a baseline such as a non-cancerous sample, including a non-cancerous matched sample from a subject known to have a tumor.

The skilled artisan will be aware that the term “hypermethylated nucleic acid” and equivalents shall be taken to mean that a plurality of CpG dinucleotides in a specific or defined region of nucleic acid is methylated. In one example, the nucleic acid is DNA.

As used herein, the term “unmodified chromatin” shall be taken to mean, for example, that a gene is expressed at a level similar to or the same as a non-cancerous cell; and/or that the level of methylation of a nucleic acid is similar or the same as a non-cancerous cell; and/or that the level of acetylation/methylation of a histone that is the same or similar to a non-cancerous cell.

As will be apparent to the skilled person from the foregoing, “less-modified” chromatin will be altered to a smaller degree and/or only be altered in some aspects compared to modified chromatin. E.g., less modified chromatin may comprise nucleic acid that is methylated to a degree less than that observed in modified chromatin. The modified chromatin may include coding or non-coding nucleic acid. Non-coding nucleic acid is understood in the art to include an intron, a 5′-untranslated region, a 3′ untranslated region, a promoter region of a genomic gene, or an intergenic region.

As used herein, the term “non-cancerous sample” shall be taken to include any sample from or including a normal or healthy cell or tissue, or a data set produced using information from a normal or healthy cell or tissue. For example, the non-cancerous sample may be selected from the group consisting of:

(i) a sample comprising a non-cancerous cell;
(ii) a sample from a normal tissue;
(iii) a sample from a healthy tissue;
(iv) an extract of any one of (i) to (iii);
(v) a data set comprising measurements of modified chromatin and/or gene expression for a healthy individual or a population of healthy individuals;
(vi) a data set comprising measurements of modified chromatin and/or gene expression for a normal individual or a population of normal individuals; and
(vii) a data set comprising measurements of the modified chromatin and/or gene expression from the subject being tested wherein the measurements are determined in a matched sample having normal cells. Preferably, the non-cancerous sample is (i) or (ii) or (v) or (vii).

As used herein, the term “subject” shall be taken to mean any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human.

### Markers

The present disclosure encompasses methods involving detecting chromatin modifications (e.g., methylation) within a LOC134466 gene and/or expression of a LOC134466 gene. In one example, the LOC134466 gene is a human gene. For the purposes of nomenclature and not limitation a sequence of a LOC134466 gene comprises a sequence set forth in SEQ ID NO: 1 or 3. A polypeptide sequence encoded by the LOC134466 gene comprises a sequence set forth in SEQ ID NO: 2. In one example, a method of the disclosure comprises detecting the level of methylation of a nucleic acid comprising or consisting of a sequence set forth in SEQ ID NO: 3 or comprising a sequence within SEQ ID NO: 3. In one example, a method of the present disclosure comprises detecting the level of methylation of a nucleic acid comprising or consisting of residues 153 to 254 of SEQ ID NO: 3.

The present disclosure encompasses a method comprising detecting one or more additional markers selected from the group consisting of ARMCX1, ICAM4, IL18, PEG3, PYCARD, SGNE1, ZNF177, HSPA2, KLF4, LTBP2, PAPLN, PARVA, PTGER, SCIN1, SPOCK2, TLE4, ZNF542, BMP6, CST6 and SOCS1. For example, the methods disclosed herein may comprise the detection of chromatin modifications (e.g., methylation) within a LOC134466 gene and any one or more of ARMCX1, ICAM4, PEG3, PYCARD and/or SGNE1, in any combination. In one example, the methods comprise the detection of chromatin modifications (e.g., methylation) within a LOC134466 gene and SGNE1. For the purposes of nomenclature and not limitation, sequences of exemplary forms of those nucleic acids and/or polypeptides are set forth in the Sequence Listing as described in the above Key to Sequence Listing.

The present disclosure also encompasses naturally-occurring variants of the foregoing markers, e.g., transcript variants and/or allelic variants. The present disclosure also encompasses sequences having at least 80% or 85% or 90% or 95% or 96% or 96% or 98% or 99% identity to the recited sequence.

The % identity of a nucleic acid or polypeptide is determined by GAP (Needleman and Wunsch, 1970) analysis (GCG program) with a gap creation penalty=5, and a gap extension penalty=0.3. The query sequence is at least 50 residues in length, and the GAP analysis aligns the two sequences over a region of at least 50 residues. Even more preferably, the query sequence is at least 100 residues in length and the GAP analysis aligns the two sequences over a region of at least 100 residues. Most preferably, the two sequences are aligned over their entire length.

### Suitable Cancers

The present disclosure encompasses the diagnosis or prognosis of any cancer. For example, the present disclosure contemplates the diagnosis or prognosis of a cancer selected from the group consisting of a breast cancer, a prostate cancer, a lung cancer, a cancer of the bronchus, a colon cancer, a rectal cancer, a cancer of the urinary bladder, a kidney cancer, a cancer of the renal pelvis, a pancreatic cancer, a head and/or neck cancer, a laryngeal cancer, a oropharyngeal cancer, a cancer of the tongue, an ovarian cancer, a thyroid cancer, a stomach cancer, a brain tumor, a cancer of the brain, a multiple myeloma, a cancer of the esophagus, a liver cancer, a cancer of the intrahepatic bile duct, a cervical cancer, a chronic lymphocytic leukemia, a soft tissue cancer, a heart cancer, a Hodgkin lymphoma, a non-Hodgkin lymphoma, a testicular cancer, a cancer of the small intestine, a cancer of the anus, a cancer of the anal canal, a cancer of the anorectum, a vulval cancer, a cancer of the gallbladder, a malignant mesothelioma, a bone cancer, a Ewing's sarcoma, an osteosarcoma, a rhabdomyosarcoma, a soft-tissue sarcoma, a cancer of the hypopharynx, a cancer of the eye, an orbital cancer, a cancer of the nasal cavity, a cancer of the middle ear, a cancer of the ureter, a gastrointestinal carinoid tumor, an adrenal cancer, a parathyroid cancer, a pituitary cancer, a gastric cancer, a hepatoma, an endometrial cancer, a uterine cancer, a gestational trophoblastic disease, a choriocarcinoma, a vaginal cancer, a fallopian tube cancer, an acute lymphocytic leukemia (ALL), an acute myelogenous leukemia (AML), a chronic lymphocytic leukemia (CLL), a chronic myelogenous leukemia (CML), a hairy cell leukemia, a myeloproliferative disorder, a mesothelioma, a non-small cell lung cancer, a small-cell lung cancer, an AIDS related lymphoma, a cutaneous T-cell lymphoma, a mucosis fungoides, a Kaposi's sarcoma and a melanoma. As will be apparent to the skilled artisan several of the cancers listed supra encompass multiple forms of cancer.

In one example, the cancer is or comprises a solid tumour. Examples of cancers contemplated by the present disclosure include ovarian cancer and/or breast cancer and/or prostate cancer and/or colon cancer.

Exemplary ovarian cancers contemplated by the present disclosure include any cancer caused by a malignant cell derived from an ovary or fallopian tube. Such a cell is derived from, for example, the epithelium of an ovary, a germ cell (e.g. an ovum) or a stromal cell of the ovary. In one example, the ovarian cancer is a non-epithelial ovarian cancer. For example, the ovarian cancer is selected from the group consisting of a Brenner's tumor, an undifferentiated tumor, a transitional cell tumor, a dysgerminoma-type germ cell tumor, a nondysgerminoma-type germ cell tumor, and a sex cord-stromal tumor. Preferably, the ovarian cancer is an epithelial ovarian cancer. For example, an epithelial ovarian cancer selected from the group consisting of a serous tumor, an endometrioid tumor, a mucinous tumor, a clear cell tumor. In one example, the ovarian cancer is serous ovarian cancer or clear cell ovarian cancer.

Exemplary breast cancers include any cancer caused by a malignant cell derived from a breast. Exemplary breast cancers include basal breast cancer, Her2 positive breast cancer, progesterone receptor positive breast cancer, estrogen receptor positive breast cancer, ductal carcinoma in situ, lobular carcinoma in situ, early breast cancer, invasive breast cancer, Paget's disease of the nipple, inflammatory breast cancer, locally advanced breast cancer and secondary breast cancer. In one example, the breast cancer is invasive breast cancer or ductal carcinoma in situ.

Exemplary prostate cancers include any cancer caused by a malignant cell derived from a prostate. Exemplary prostate cancers include any adenocarcinoma small cell undifferentiated carcinoma and mucinous (colloid) cancer of the prostate tissue. This term encompasses primary cancer, relapsed forms of cancer and metastasized cancers.

### Diagnostic Assay Formats

### I. Detection of Methylation of Nucleic Acid

The present inventors have demonstrated modified levels of methylation at specific loci in cancer cells compared to control non-cancerous cells. Accordingly, a method for detecting modified chromatin described herein shall be taken to include detecting the level of methylation of nucleic acid and/or hypermethylation of nucleic acid in the chromatin. Suitable methods for the detection of methylation levels are known in the art and/or described herein.

The term “methylation of nucleic acid” shall be taken to mean the addition of a methyl group by the action of a DNA methyl transferase enzyme to a CpG island of nucleic acid, e.g., genomic DNA. As described herein, there are several methods known to those skilled in the art for determining the level or degree of methylation of nucleic acid. By “enhanced” is meant that there are a significantly larger number of methylated CpG dinucleotides in the subject diagnosed than in a suitable control sample. The present invention is not to be limited by a precise number of methylated residues that are considered to be diagnostic of cancer in a subject, because some variation between patient samples will occur. The present invention is also not limited by positioning of the methylated residue.

The term “hypermethylated nucleic acid” and equivalents shall be taken to mean that a plurality of CpG dinucleotides in a specific or defined region of nucleic acid is methylated.

In one example, the degree of methylation is determined in a nucleic acid comprising a nucleotide sequence set forth in SEQ ID NO: 2.

a. Probe or Primer Design and/or Production

Several methods described herein for the diagnosis of a cancer use one or more probes and/or primers. Methods for designing probes and/or primers for use in, for example, PCR or hybridization are known in the art and described, for example, in Dieffenbach and Dveksler (Eds) (In: PCR Primer: A Laboratory Manual, Cold Spring Harbor Laboratories, NY, 1995). Furthermore, several software packages are publicly available that design optimal probes and/or primers for a variety of assays, e.g. Primer 3 available from the Center for Genome Research, Cambridge, Mass., USA.

The potential use of the probe or primer should be considered during its design. For example, should the probe or primer be produced for use in, for example, a methylation specific PCR or ligase chain reaction (LCR) assay the nucleotide at the 3′ end (or 5′ end in the case of LCR) should correspond to a methylated nucleotide in a nucleic acid.

Probes and/or primers useful for detection of a marker associated with a cancer are assessed, for example, to determine those that do not form hairpins, self-prime or form primer dimers (e.g. with another probe or primer used in a detection assay).

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references cited therein.

Probes comprising locked nucleic acid (LNA) are synthesized as described, for example, in Nielsen et al, J. Chem. Soc. Perkin Trans., 1: 3423, 1997; Singh and Wengel, Chem. Commun. 1247, 1998. While, probes comprising peptide-nucleic acid (PNA) are synthesized as described, for example, in Egholm et al., Am. Chem. Soc., 114: 1895, 1992; Egholm et al., Nature, 365: 566, 1993; and Orum et al., Nucl. Acids Res., 21: 5332, 1993.

b. Methylation-Sensitive Endonuclease Digestion of DNA

In one example, the enhanced methylation in a sample is determined using a process comprising treating the nucleic acid with an amount of a methylation-sensitive restriction endonuclease enzyme under conditions sufficient for nucleic acid to be digested and then detecting the fragments produced. Exemplary methylation-sensitive endonucleases include, for example, HpaI or HpaII.

In one example, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization (Kawai et al., Mol. Cell. Biol. 14, 7421-7427, 1994; Gonzalgo et al., Cancer Res. 57, 594-599, 1997).

The term “selectively hybridizable” means that the probe is used under conditions where a target nucleic acid hybridizes to the probe to produce a signal that is significantly above background (i.e., a high signal-to-noise ratio). The intensity of hybridization is measured, for example, by radiolabeling the probe, e.g. by incorporating [α-³⁵S] and/or [α-³²P]dNTPs, [γ-³²P]ATP, biotin, a dye ligand (e.g., FAM or TAMRA), a fluorophore, or other suitable ligand into the probe prior to use and then detecting the ligand following hybridization.

The skilled artisan will be aware that optimum hybridization reaction conditions should be determined empirically for each probe, although some generalities can be applied. Preferably, hybridizations employing short oligonucleotide probes are performed at low to medium stringency.

For the purposes of defining the level of stringency to be used in these diagnostic assays, a low stringency is defined herein as being a hybridization and/or a wash carried out in about 6×SSC buffer and/or about 0.1% (w/v) SDS at about 28° C. to about 40° C., or equivalent conditions. A moderate stringency is defined herein as being a hybridization and/or washing carried out in about 2×SSC buffer and/or about 0.1% (w/v) SDS at a temperature in the range of about 45° C. to about 65° C., or equivalent conditions.

In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used and/or constituents of any buffer used in a hybridization. For example, formamide reduces the melting temperature of a probe or primer in a hybridization or an amplification reaction.

Conditions for specifically hybridizing nucleic acid, and conditions for washing to remove non-specific hybridizing nucleic acid, are understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

In accordance with the present example, a difference in the fragments produced for the test sample and a negative control sample is indicative of the subject having cancer. Similarly, in cases where the control sample comprises data from a tumor, cancer tissue or a cancerous cell or pre-cancerous cell, similarity, albeit not necessarily absolute identity, between the test sample and the control sample is indicative of a positive diagnosis (i.e. cancer).

In an alternative example, the fragments produced by the restriction enzyme are detected using an amplification system, such as, for example, polymerase chain reaction (PCR), rolling circle amplification (RCA), inverse polymerase chain reaction (iPCR), in situ PCR (Singer-Sam et al., Nucl. Acids Res. 18, 687, 1990), strand displacement amplification (SDA) or cycling probe technology.

Methods of PCR are known in the art and described, for example, by McPherson et al., PCR: A Practical Approach. (series eds, D. Rickwood and B. D. Hames), IRL Press Limited, Oxford. pp 1-253, 1991 and by Dieffenbach (ed) and Dveksler (ed) (In: PCR Primer: A Laboratory Manual, Cold Spring Harbour Laboratories, NY, 1995), the contents of which are each incorporated in their entirety by way of reference. Generally, for PCR two non-complementary nucleic acid primer molecules comprising at least about 18 nucleotides in length, and more preferably at least 20-30 nucleotides in length are hybridized to different strands of a nucleic acid template molecule at their respective annealing sites, and specific nucleic acid molecule copies of the template that intervene the annealing sites are amplified enzymatically. Amplification products may be detected, for example, using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA).

Strand displacement amplification (SDA) utilizes oligonucleotide primers, a DNA polymerase and a restriction endonuclease to amplify a target sequence. The oligonucleotides are hybridized to a target nucleic acid and the polymerase is used to produce a copy of the region intervening the primer annealing sites. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Cycling Probe Technology uses a chimeric synthetic primer that comprises DNA-RNA-DNA that is capable of hybridizing to a target sequence. Upon hybridization to a target sequence the RNA-DNA duplex formed is a target for RNaseH thereby cleaving the primer. The cleaved primer is then detected, for example, using mass spectrometry or electrophoresis.

For primers that flank or are adjacent to a methylation-sensitive endonuclease recognition site, it is preferred that such primers flank only those sites that are hypermethylated in cancer to ensure that a diagnostic amplification product is produced. In this regard, an amplification product will only be produced when the restriction site is not cleaved, i.e., when it is methylated. Accordingly, detection of an amplification product indicates that the CpG dinucleotide/s of interest is/are methylated.

This form of analysis may be used to determine the methylation status of a plurality of CpG dinucleotides provided that each dinucleotide is within a methylation sensitive restriction endonuclease site.

In these methods, one or more of the primers may be labeled with a detectable marker to facilitate rapid detection of amplified nucleic acid, for example, a fluorescent label (e.g. Cy5 or Cy3) or a radioisotope (e.g. ³²P).

The amplified nucleic acids are generally analyzed using, for example, non-denaturing agarose gel electrophoresis, non-denaturing polyacrylamide gel electrophoresis, mass spectrometry, liquid chromatography (e.g. HPLC or dHPLC), or capillary electrophoresis. (e.g. MALDI-TOF). High throughput detection methods, such as, for example, matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or DNA chip technology (e.g., WO98/49557; WO 96/17958; Fodor et al., Science 767-773, 1991; U.S. Pat. No. 5,143,854; and U.S. Pat. No. 5,837,832, the contents of which are all incorporated herein by reference).

Alternatively, amplification of a nucleic acid may be continuously monitored using a melting curve analysis method as described herein and/or in, for example, U.S. Pat. No. 6,174,670, which is incorporated herein by reference.

c. Selective Mutagenesis of Non-Methylated DNA

In an alternative example of the present disclosure, the enhanced methylation in a subject sample is determined using a process comprising treating the nucleic acid with an amount of a compound that selectively mutates a non-methylated cytosine residue within a CpG dinucleotide under conditions sufficient to induce mutagenesis.

Exemplary compounds mutate cytosine to uracil or thymidine, such as, for example, a salt of bisulfite, e.g., sodium bisulfite or potassium bisulfite (Frommer et al., Proc. Natl. Acad. Sci. USA 89, 1827-1831, 1992). Bisulfite treatment of DNA is known to distinguish methylated from non-methylated cytosine residues, by mutating cytosine residues that are not protected by methylation, including cytosine residues that are not within a CpG dinucleotide or that are positioned within a CpG dinucleotide that is not subject to methylation.

c(i) Sequence Based Detection

In one example, the presence of one or more mutated nucleotides or the number of mutated sequences is determined by sequencing mutated DNA. One form of analysis comprises amplifying mutated nucleic acid or methylated nucleic acid using an amplification reaction described herein, for example, PCR. The amplified product is then directly sequenced or cloned and the cloned product sequenced. Methods for sequencing DNA are known in the art and include for example, the dideoxy chain termination method or the Maxam-Gilbert method (see Sambrook et al., Molecular Cloning, A Laboratory Manual (2nd Ed., CSHP, New York 1989) or Zyskind et al., Recombinant DNA Laboratory Manual, (Acad. Press, 1988)).

As the treatment of nucleic acid with a compound, such as, for example, bisulfite results in non-methylated cytosines being mutated to uracil or thymidine, analysis of the sequence determines the presence or absence of a methylated nucleotide. For example, by comparing the sequence obtained using a control sample or a sample that has not been treated with bisulfite, or the known nucleotide sequence of the region of interest with a treated sample facilitates the detection of differences in the nucleotide sequence. Any thymine residue detected at the site of a cytosine in the treated sample compared to a control or untreated sample may be considered to be caused by mutation as a result of bisulfite treatment. Suitable methods for the detection of methylation using sequencing of bisulfite treated nucleic acid are described, for example, in Frommer et al., Proc. Natl. Acad. Sci. USA 89: 1827-1831, 1992 or Clark et al., Nucl. Acids Res. 22: 2990-2997, 1994. One example of a commercially available kit for carrying out such methods is the CpGenome™ DNA modification Kit (Millipore). Other suitable kits are available from MDX Health SA (Belgium).

In another example, the presence of a mutated or non-mutated nucleotide in a bisulfite treated sample is detected using pyrosequencing, such as, for example, as described in Uhlmann et al., Electrophoresis, 23: 4072-4079, 2002. Essentially this method is a form of real-time sequencing that uses a primer that hybridizes to a site adjacent or close to the site of a cytosine that is methylated in a cancer cell. Following hybridization of the primer and template in the presence of a DNA polymerase each of four modified deoxynucleotide triphosphates are added separately according to a predetermined dispensation order. Only an added nucleotide that is complementary to the bisulfite treated sample is incorporated and inorganic pyrophosphate (PPi) is liberated. The PPi then drives a reaction resulting in production of detectable levels of light. Such a method allows determination of the identity of a specific nucleotide adjacent to the site of hybridization of the primer.

A related method for determining the sequence of a bisulfite treated nucleotide is methylation-sensitive single nucleotide primer extension (Me-SnuPE) or SNaPmeth. Suitable methods are described, for example, in Gonzalgo and Jones Nucl. Acids Res., 25: 2529-2531 or Uhlmann et al., Electrophoresis, 23: 4072-4079, 2002.

Clearly other high throughput sequencing methods are encompassed by the present invention. Such methods include, for example, solid phase minisequencing (as described, for example, in Syvämen et al, Genomics, 13: 1008-1017, 1992), or minisequencing with FRET (as described, for example, in Chen and Kwok, Nucleic Acids Res. 25: 347-353, 1997).

c(ii) Restriction Endonuclease-Based Assay Format

In one example, the presence of a non-mutated sequence is detected using combined bisulfite restriction analysis (COBRA) essentially as described in Xiong and Laird, Nucl. Acids Res., 25: 2532-2534, 2001. This method exploits the differences in restriction enzyme recognition sites between methylated and unmethylated nucleic acid after treatment with a compound that selectively mutates a non-methylated cytosine residue, e.g., bisulfite.

Following bisulfite treatment a region of interest comprising one or more CpG dinucleotides that are methylated in a cancer cell and are included in a restriction endonuclease recognition sequence is amplified using an amplification reaction described herein, e.g., PCR. The amplified product is then contacted with the restriction enzyme that cleaves at the site of the CpG dinucleotide for a time and under conditions sufficient for cleavage to occur. A restriction site may be selected to indicate the presence or absence of methylation. For example, the restriction endonuclease TagI cleaves the sequence TCGA, following bisulfite treatment of a non-methylated nucleic acid the sequence will be TTGA and, as a consequence, will not be cleaved. The digested and/or non-digested nucleic acid is then detected using a detection means known in the art, such as, for example, electrophoresis and/or mass spectrometry. The cleavage or non-cleavage of the nucleic acid is indicative of cancer in a subject.

Clearly, this method may be employed in either a positive read-out or negative read-out system for the diagnosis of a cancer.

(c)(iii) Positive Read-Out Assay Format

In one example, the assay format of the invention comprises a positive read-out system in which DNA from a cancer sample that has been treated, for example, with bisulfite is detected as a positive signal. For example, the non-hypermethylated DNA from a healthy or normal control subject is not detected or only weakly detected.

In one example, the enhanced methylation in a subject sample is determined using a process comprising:
(i) treating the nucleic acid with an amount of a compound that selectively mutates a non-methylated cytosine residue under conditions sufficient to induce mutagenesis thereby producing a mutated nucleic acid;
(ii) hybridizing a nucleic acid to a probe or primer comprising a nucleotide sequence that is complementary to a sequence comprising a methylated cytosine residue under conditions such that selective hybridization to the non-mutated nucleic acid occurs; and
(iii) detecting the selective hybridization.

In this context, the term “selective hybridization” in this context means that hybridization of a probe or primer to the non-mutated nucleic acid occurs at a higher frequency or rate, or has a higher maximum reaction velocity, than hybridization of the same probe or primer to the corresponding mutated sequence. Preferably, the probe or primer does not hybridize or detectably hybridize (e.g., does not hybridize at a level significantly above background) to the non-methylated sequence carrying the mutation(s) under the reaction conditions used.

In one example, the hybridization is detected using Southern, dot blot, slot blot or other nucleic acid hybridization means (Kawai et al., Mol. Cell. Biol. 14, 7421-7427, 1994; Gonzalgo et al., Cancer Res. 57, 594-599, 1997). Subject to appropriate probe selection, such assay formats are generally described herein above and apply mutatis mutandis to the presently described selective mutagenesis approach.

In one example, a ligase chain reaction format is employed to distinguish between a mutated and non-mutated nucleic acid. Ligase chain reaction (described in EP 320,308 and U.S. Pat. No. 4,883,750) uses at least two oligonucleotide probes that anneal to a target nucleic acid in such a way that they are juxtaposed on the target nucleic acid such that they can be linked using a ligase. The probes that are not ligated are removed by modifying the hybridization stringency. In this respect, probes that have not been ligated will selectively hybridize under lower stringency hybridization conditions than probes that have been ligated. Accordingly, the stringency of the hybridization can be increased to a stringency that is at least as high as the stringency used to hybridize the longer probe, and preferably at a higher stringency due to the increased length contributed by the shorter probe following ligation. One exemplary method melts the target-probe duplex, elute the dissociated probe and confirm that is has been ligated, e.g., by determining its length using electrophoresis, mass spectrometry, nucleotide sequence analysis, gel filtration, or other means known to the skilled artisan.

Methylation specific microarrays (MSO) are also useful for differentiating between a mutated and non-mutated sequence. A suitable method is described, for example, in Adorjan et al, Nucl. Acids Res., 30: e21, 2002. MSO uses nucleic acid that has been treated with a compound that selectively mutates a non-methylated cytosine residue (e.g., bisulfite) as template for an amplification reaction that amplifies both mutant and non-mutated nucleic acid. The amplification is performed with at least one primer that comprises a detectable label, such as, for example, a fluorophore, e.g., Cy3 or Cy5. The labeled amplification products are then hybridized to oligonucleotides on the microarray under conditions that enable detection of single nucleotide differences. Following washing to remove unbound amplification product, hybridization is detected using, for example, a microarray scanner. Not only does this method allow for determination of the methylation status of a large number of CpG dinucleotides, it is also semi-quantitative, enabling determination of the degree of methylation at each CpG dinucleotide analyzed. As there may be some degree of heterogeneity of methylation in a single sample, such quantification may assist in the diagnosis of cancer.

In an alternative example, the hybridization is detected using an amplification system. In methylation-specific PCR formats (MSP; Herman et al. Proc. Natl. Acad. Sci. USA 93: 9821-9826, 1992), the hybridization is detection using a process comprising amplifying the bisulfite-treated DNA. By using one or more probe or primer that anneals specifically to the unmutated sequence under moderate and/or high stringency conditions an amplification product is only produced using a sample comprising a methylated nucleotide.

Any amplification assay format described herein can be used, such as, for example, polymerase chain reaction (PCR), rolling circle amplification (RCA), inverse polymerase chain reaction (iPCR), in situ PCR (Singer-Sam et al., Nucl. Acids Res. 18, 687, 1990), strand displacement amplification, or cycling probe technology.

PCR techniques have been developed for detection of gene mutations (Kuppuswamy et al., Proc. Natl. Acad. Sci. USA 88:1143-1147, 1991) and quantitation of allelic-specific expression (Szabo and Maim, Genes Dev. 9: 3097-3108, 1995; and Singer-Sam et al., PCR Methods Appl. 1: 160-163, 1992). Such techniques use internal primers, which anneal to a PCR-generated template and terminate immediately 5′ of the single nucleotide to be assayed. Such as format is readily combined with ligase chain reaction as described herein above.

Methylation-specific melting-curve analysis (essentially as described in Worm et al., Clin. Chem., 47: 1183-1189, 2001) is also contemplated by the present invention. This process exploits the difference in melting temperature in amplification products produced using bisulfite treated methylated or unmethylated nucleic acid. In essence, non-discriminatory amplification of a bisulfite treated sample is performed in the presence of a fluorescent dye that specifically binds to double stranded DNA (e.g., SYBR Green I). By increasing the temperature of the amplification product while monitoring fluorescence the melting properties and thus the sequence of the amplification product is determined. A decrease in the fluorescence reflects melting of at least a domain in the amplification product. The temperature at which the fluorescence decreases is indicative of the nucleotide sequence of the amplified nucleic acid, thereby permitting the nucleotide at the site of one or more CpG dinucleotides to be determined. As the sequence of the nucleic acids amplified using the present invention

The present disclosure also encompasses the use of real-time quantitative forms of PCR, such as, for example, TaqMan (Holland et al., Proc. Natl. Acad. Sci. USA, 88, 7276-7280, 1991; Lee et al., Nucleic Acid Res. 21, 3761-3766, 1993) to perform this embodiment. For example, the MethylLight method of Eads et al., Nucl. Acids Res. 28: E32, 2000 uses a modified TaqMan assay to detect methylation of a CpG dinucleotide.

Alternatively, rather than using a labeled probe that requires cleavage, a probe, such as, for example, a Molecular Beacon™ is used (see, for example, Mhlang and Malmberg, Methods 25: 463-471, 2001). Molecular beacons are single stranded nucleic acid molecules with a stem-and-loop structure. The loop structure is complementary to the region surrounding the one or more CpG dinucleotides that are methylated in a cancer sample and not in a control sample. The stem structure is formed by annealing two “arms” complementary to each other, which are on either side of the probe (loop). A fluorescent moiety is bound to one arm and a quenching moiety that suppresses any detectable fluorescence when the molecular beacon is not bound to a target sequence is bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base is determined by the level of fluorescence detected. Such an assay facilitates detection of one or more unmutated sites (i.e. methylated nucleotides) in a nucleic acid.

As exemplified herein, another amplification based assay useful for the detection of a methylated nucleic acid following treatment with a compound that selectively mutates a non-methylated cytosine residue makes use of headloop PCR technology (e.g., as described in published PCT Application No. PCT/AU03/00244; WO 03/072810). This form of amplification uses a probe or primer that comprises a region that binds to a nucleic acid and is capable of amplifying nucleic acid in an amplification reaction whether the nucleic acid is methylated or not. The primer additionally comprises a region that is complementary to a portion of the amplified nucleic acid enabling this region of the primer to hybridize to the amplified nucleic acid incorporating the primer thereby forming a hairpin. The now 3′ terminal nucleotide/s of the annealed region (i.e. the most 5′ nucleotide/s of the primer) hybridize to the site of one or more mutated cytosine residues (i.e., unmethylated in nucleic acid from a cancer subject). Accordingly, this facilitates self priming of amplification products from unmethylated nucleic acid, the thus formed hairpin structure blocking further amplification of this nucleic acid. In contrast, the complementary region may or may not by capable of hybridizing to an amplification product from methylated (mutated) nucleic acid, but is unable to “self prime” thereby enabling further amplification of this nucleic acid (e.g., by the inability of the now 3′ nucleotide to hybridize to the amplification product). This method may be performed using a melting curve analysis method to determine the amount of methylated nucleic acid in a biological sample from a subject.

Other amplification based methods for detecting methylated nucleic acid following treatment with a compound that selectively mutates a non-methylated cytosine residue include, for example, methylation-specific single stranded conformation analysis (MS-SSCA) (Bianco et al., Hum. Mutat., 14: 289-293, 1999), methylation-specific denaturing gradient gel electrophoresis (MS-DGGE) (Abrams and Stanton, Methods Enzymol., 212: 71-74, 1992) and methylation-specific denaturing high-performance liquid chromatography (MS-DHPLC) (Deng et al, Chin. J. Cancer Res., 12: 171-191, 2000). Each of these methods use different techniques for detecting nucleic acid differences in an amplification product based on differences in nucleotide sequence and/or secondary structure. Such methods are clearly contemplated by the present invention.

(c)(iv) Negative Read-Out Assays

In an alternative example, the assay format comprises a negative read-out system in which reduced methylation of DNA from a healthy/normal control sample is detected as a positive signal and preferably, methylated DNA from a cancer sample is not detected or is only weakly detected.

In one example, the reduced methylation is determined using a process comprising:
(i) treating the nucleic acid with an amount of a compound that selectively mutates a non-methylated cytosine residue within a CpG island under conditions sufficient to induce mutagenesis thereby producing a mutated nucleic acid;
(ii) hybridizing the nucleic acid to a probe or primer comprising a nucleotide sequence that is complementary to a sequence comprising the mutated cytosine residue under conditions such that selective hybridization to the mutated nucleic acid occurs; and
(iii) detecting the selective hybridization.

In this context, the term “selective hybridization” means that hybridization of a probe or primer to the mutated nucleic acid occurs at a higher frequency or rate, or has a higher maximum reaction velocity, than hybridization of the same probe or primer to the corresponding non-mutated sequence. In one example, the probe or primer does not hybridize or detectably hybridize to the methylated sequence (or non-mutated sequence) under the reaction conditions used.

The skilled artisan will be able to adapt a positive read-out assay described above to a negative read-out assay, e.g., by producing a probe or primer that selectively hybridizes to non-mutated DNA rather than mutated DNA.

### d) Additional Method Steps

The methods disclosed herein may further comprise one or more steps of enriching methylated DNA in a sample. Thus, the methods disclosed herein may further comprise one or more steps of isolating methylated DNA from a sample. The enrichment/isolation step may be performed prior to or concomitant with any other step in the method for detecting the level of methylation of a marker as disclosed herein.

Any suitable enriching/isolating step known in the art may be used. For example, the methods disclosed herein may comprise a step of enriching methylated DNA in a sample using a commercially available kit such as the CpG MethylQuest DNA Isolation Kit (Millipore), which provides a recombinant protein comprising the methyl binding domain (MBD) of the mouse MBD2b protein fused to a glutathione-S-transferase (GST) protein from S. japonicum via a linker containing a thrombin cleavage site, the recombinant protein being immobilized to a magnetic bead. The MBD binds to methylated CpG sites with high affinity and in a sequence-independent manner, thereby allowing enrichment of methylated DNA in a sample.

It will be appreciated that alternative or additional methods known in the art for enrichment/isolation of methylated DNA in a sample can be used in the methods disclosed herein.

A method disclosed herein according to any example may also comprise selecting a patient based on the result of a method disclosed herein and performing an additional diagnostic method or recommending performance of an additional diagnostic method. For example, for a patient diagnosed as suffering from ovarian cancer, the additional diagnostic method may be an ultrasound. For a patient diagnosed as suffering from colorectal cancer, the additional diagnostic method may be a colonoscopy or a biopsy.

### II. Detection of Reduced Gene Expression

The present inventors have also demonstrated that the level of expression of nucleic acids within any of a number of loci described herein is reduced in cancer subjects and in cancer cell lines.

### Nucleic Acid Detection

In one example, the level of expression is determined by detecting the level of mRNA transcribed from a gene or pseudogene.

In one example, the mRNA is detected by hybridizing a nucleic acid probe or primer capable of specifically hybridizing to a transcript of a gene or pseudogene described herein to a nucleic acid in a biological sample derived from a subject and detecting the hybridization by a detection means. Preferably, the detection means is an amplification reaction, or a nucleic acid hybridization reaction, such as, for example, as described herein.

In this context, the term “selective hybridization” means that hybridization of a probe or primer to the transcript occurs at a higher frequency or rate, or has a higher maximum reaction velocity, than hybridization of the same probe or primer to any other nucleic acid. Preferably, the probe or primer does not hybridize to another nucleic acid at a detectable level under the reaction conditions used.

As transcripts of a gene or pseudogene described herein are detected using mRNA or cDNA derived therefrom, assays that detect changes in mRNA are preferred (e.g. Northern hybridization, RT-PCR, NASBA, TMA or ligase chain reaction).

Methods of RT-PCR are known in the art and described, for example, in Dieffenbach (ed) and Dveksler (ed) (In: PCR Primer: A Laboratory Manual, Cold Spring Harbour Laboratories, NY, 1995). Essentially, this method comprises performing a PCR reaction using cDNA produced by reverse transcribing mRNA from a cell using a reverse transcriptase. Methods of PCR described supra are to be taken to apply mutatis mutandis to this embodiment of the invention.

Similarly LCR may be performed using cDNA. Preferably, one or more of the probes or primers used in the reaction specifically hybridize to the transcript of interest. Method of LCR are described supra and are to be taken to apply mutatis mutandis to this embodiment of the invention.

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

The present disclosure also contemplates the use of a microarray to determine the level of expression of one or more nucleic acids described herein. Such a method enables the detection of a number of different nucleic acids, thereby providing a multi-analyte test and improving the sensitivity and/or accuracy of the diagnostic assay of the invention.

### Polypeptide Detection

In an alternative example, the level of expression is determined by detecting the level of a protein encoded by a nucleic acid within a gene or pseudogene described herein.

In this respect, the present invention is not necessarily limited to the detection of a protein comprising the specific amino acid sequence recited herein. Rather, the present invention encompasses the detection of variant sequences (e.g., having at least about 80% or 90% or 95% or 98% amino acid sequence identity) or the detection of an immunogenic fragment or epitope of said protein.

The amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one example, the assay used to determine the amount or level of a protein is a semi-quantitative assay. In another example, the assay used to determine the amount or level of a protein in a quantitative assay. As will be apparent from the preceding description, such an assay may require the use of a suitable control, e.g. from a normal individual or matched normal control.

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide). An antibody that specifically binds to a protein described herein is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

In another form, an ELISA or FLISA comprises immobilizing an antibody or ligand that specifically binds a protein described supra on a solid matrix, such as, for example, a membrane, a polystyrene or polycarbonate microwell, a polystyrene or polycarbonate dipstick or a glass support. A sample is then brought into physical relation with said antibody, and the polypeptide is bound or ‘captured’. The bound protein is then detected using a labeled antibody. For example, a labeled antibody that binds to an epitope that is distinct from the first (capture) antibody is used to detect the captured protein. Alternatively, a third labeled antibody can be used that binds the second (detecting) antibody.

In another example, the presence or level of a protein is detected in a body fluid using, for example, a biosensor instrument (e.g., BIAcore™, Pharmacia Biosensor, Piscataway, N.J.). In such an assay, an antibody or ligand that specifically binds a protein is immobilized onto the surface of a receptor chip. For example, the antibody or ligand is covalently attached to dextran fibers that are attached to gold film within the flow cell of the biosensor device. A test sample is passed through the cell. Any antigen present in the body fluid sample, binds to the immobilized antibody or ligand, causing a change in the refractive index of the medium over the gold film, which is detected as a change in surface plasmon resonance of the gold film.

In another example, the presence or level of a protein or a fragment or epitope thereof is detected using a protein and/or antibody chip. To produce such a chip, an antibody or ligand that binds to the antigen of interest is bound to a solid support such as, for example glass, polycarbonate, polytetrafluoroethylene, polystyrene, silicon oxide, gold or silicon nitride. This immobilization is either direct (e.g. by covalent linkage, such as, for example, Schiff's base formation, disulfide linkage, or amide or urea bond formation) or indirect.

To bind a protein to a solid support it is often necessary to treat the solid support so as to create chemically reactive groups on the surface, such as, for example, with an aldehyde-containing silane reagent or the calixcrown derivatives described in Lee et al, Proteomics, 3: 2289-2304, 2003. A streptavidin chip is also useful for capturing proteins and/or peptides and/or nucleic acid and/or cells that have been conjugated with biotin (e.g. as described in Pavlickova et al., Biotechniques, 34: 124-130, 2003). Alternatively, a peptide is captured on a microfabricated polyacrylamide gel pad and accelerated into the gel using microelectrophoresis as described in, Arenkov et al. Anal. Biochem. 278:123-131, 2000.

Other assay formats are also contemplated, such as flow-trough immunoassays (PCT/AU2002/01684), a lateral flow immunoassay (US20040228761, US20040248322 or US20040265926), a fluorescence polarization immunoassay (FPIA) (U.S. Pat. Nos. 4,593,089, 4,492,762, 4,668,640, and 4,751,190), a homogeneous microparticles immunoassay (“HMI”) (e.g., U.S. Pat. Nos. 5,571,728, 4,847,209, 6,514,770, and 6,248,597) or a chemiluminescent microparticle immunoassay (“CMIA”).

### III Multiplex Assay Formats

The present disclosure also contemplates multiplex or multianalyte format assays to improve the accuracy or specificity of a diagnosis of cancer. Such assays may also improve the population coverage by an assay.

Methods for determining the sensitivity of an assay will be apparent to the skilled artisan. For example, an assay described herein is used to analyze a population of test subjects to determine those that will develop cancer. Post-mortem analysis is then used to determine those subjects that did actually determine cancer. The number of “true positives” (i.e., subjects that developed cancer and were positively identified using the method of the disclosure) and “true negatives” (i.e., subjects that did not develop cancer and were not identified using the method of the disclosure) are determined.

Sensitivity of the assay is then determined by the following formula:

No. of true positives/(No. of true positives+No. of true negatives).

In one example, a method of the invention has a high degree of sensitivity. For example, in a test population of individuals, the assay detects at least about 50% of subjects developing or suffering from cancer, for example, at least about 60% of subjects developing or suffering from cancer, for example, at least about 65% of subjects developing or suffering from cancer, for example, at least about 70% of subjects developing or suffering from cancer, for example, at least about 75% of subjects developing or suffering from cancer, for example, at least about 80% of subjects developing or suffering from cancer, for example, at least about 85% of subjects developing or suffering from cancer, for example, at least about 90% of subjects developing or suffering from cancer, for example, at least about 95% of subjects developing or suffering from cancer.

Specificity is determined by the following formula:

No. of true negatives/(No. of true negatives+No. of false positives).

An exemplary multiplex assay comprises, for example, detecting hypermethylation of one or more CpG dinucleotides in a LOC134466 pseudogene and in any one or more of ARMCX1, ICAM4, IL18, PEG3, PYCARD, SGNE1 and ZNF177. In one example, the method comprises detecting the level of methylation of one or more CpG dinucleotides in a LOC134466 pseudogene and in each of ARMCX1, ICAM4, PEG3, PYCARD and SGNE1. In another example, the method comprises detecting the level of methylation of one or more CpG dinucleotides in a LOC134466 pseudogene and in SGNE1.

The multiplex assay of the invention is not to be limited to the detection of methylation at a single CpG dinucleotide within a region of interest. Rather, the present disclosure contemplates detection of methylation at a sufficient number of CpG dinucleotides in each nucleic acid to provide a diagnosis. For example, the invention contemplates detection of methylation at 1 or 2 or 3 or 4 or 5 or 7 or 9 or 10 or 15 or 20 or 25 or 30 CpG dinculeotides in each nucleic acid.

As will be apparent from the foregoing description a methylation specific microarray is amenable to such high density analysis. Previously, up to 232 CpG dinucleotides have been analyzed using such a microarray (Adorján et al., Nucl. Acids Res. 30: e21, 2002).

In another example, the method determines the level of expression of LOC134466 and any one or more of ARMCX1, ICAM4, IL18, PEG3, PYCARD, SGNE1 and ZNF177 to diagnose cancer. For example, the method may comprise detecting the level of expression of LOC134466 and each of ARMCX1, ICAM4, PEG3, PYCARD and SGNE1. In another example, the method comprises detecting the level of expression of LOC134466 and SGNE1. The level of mRNA or protein may be detected. Alternatively, the level of mRNA transcribed from one or more genes and the level of one or more proteins expressed by the same or different genes may be determined.

Each of the previously described detection techniques can be used independently of one another to diagnose cancer. Accordingly, a single sample may be analyzed to determine the level of methylation of one or more CpG dinculeotides in one or more nucleic acids and the level of expression of one or more nucleic acids and/or proteins is also determined. In accordance with this example, enhanced methylation and reduced gene expression is indicative of cancer.

Based on the teachings provided herein, a variety of combinations of assays will be apparent to the skilled artisan.

The present disclosure also contemplates the use of a known diagnostic assay in combination with an assay described herein. For example, the level of serum PSA and/or CA125 may be determined in combination with an assay described herein to diagnose cancer (such as prostate cancer and/or ovarian cancer). Alternatively, a mutation in a BRCA gene and an assay described herein may be used to diagnose breast cancer.

### Samples

A sample useful for the method of the present invention is, for example, from a tissue suspected of comprising a cancer or cancer cell. For example, the cell is from a region of a tissue thought to comprise a cancer or cancer cell. This does not exclude cells that have originated in a particular tissue but are isolated from a remote source, for example, a body fluid or a stool sample in the case of a colon cancer or urine in the case of a urogenital cancer.

The sample may be taken from a subject suspected of having or being at risk of developing cancer. For example, the subject may have a family history of cancer, may have been subjected to tests identifying elevated levels of CA125 (which, in one example, may be deemed to indicate an increased likelihood of having or being susceptible to developing ovarian cancer), may have been subjected to tests identifying elevated levels of PSA (which, in one example, may be deemed to indicate an increased likelihood of having or being susceptible to developing prostate cancer), may have been subjected to a fecal occult blood test (FOBT), a fecal immunochemical test (FIT) and/or a stool DNA test indicating an increased likelihood of having or being susceptible to developing colon cancer, or may have been subjected to any other test for detecting and/or determining the likelihood of developing any form of cancer. The sample may be taken from a subject who has been subjected to any combination of any known test for detecting and/or determining the likelihood of developing any form of cancer. Alternatively, the sample may be taken from a subject not previously suspected of having cancer.

In one example, the sample comprises a body fluid or a derivative of a body fluid or a body secretion. For example, the body fluid is selected from the group consisting of whole blood, urine, saliva, breast milk, pleural fluid, sweat, tears and mixtures thereof. An example of a derivative of a body fluid is selected from the group consisting of plasma, serum or buffy coat fraction. In one example, the sample comprises a whole blood sample, a serum sample or a plasma sample.

In one example DNA is isolated from either; whole blood, plasma, serum, peripheral blood mononucleated cells (PBMC) or enriched epithelial cells derived from the blood of patients diagnosed with ovarian cancer, patients undergoing oopherectomy for non ovarian malignancies or healthy controls. DNA may then be bisulfite converted and gene-specific methylated sequences may be detected by either; methylation specific headloop suppression PCR, MALDI-TOF mass spectrometry (sequenom) or other bisulfite based PCR assay.

In another example, a body secretion comprises stool.

Preferably, the sample comprises a nucleated cell or an extract thereof. More preferably, the sample comprises a cancer cell or an extract thereof.

In another example, the sample comprises nucleic acid and/or protein from a cancer cell. The nucleic acid and/or protein may be separate need not be isolated with a cell, but rather may be from, for example, a lysed cell.

As the present invention is particularly useful for the early detection of cancer in the medium to long term, the term cancer cell is not to be limited by the stage of a cancer in the subject from which said cancer cell is derived (i.e. whether or not the patient is in remission or undergoing disease recurrence or whether or not the cancer is a primary tumor or the consequence of metastases). Nor is the term “cancer cell” to be limited by the stage of the cell cycle of said cancer cell.

In one example, the sample comprises a cell or a plurality of cells derived from a tissue selected from the group consisting of a breast, a colon, an ovary and a prostate.

In one example, the biological sample has been isolated previously from the subject. In accordance with this example, a method of the present disclosure is performed ex vivo. In such cases, the sample may be processed or partially processed into a nucleic acid sample that is substantially free of contaminating protein. All such examples are encompassed by the present disclosure.

Methods for isolating a sample from a subject are known in the art and include, for example, surgery, biopsy, collection of a body fluid, for example, by paracentesis or thoracentesis or collection of, for example, blood or a fraction thereof. All such methods for isolating a biological sample shall be considered to be within the scope of providing or obtaining a sample.

For example, a sample from a prostate, the sample is collected, for example, by surgery (e.g., a radical prostatectomy) or a biopsy. In the case of a breast cancer, a sample is collected, for example, using a fine needle aspiration biopsy, a core needle biopsy, or a surgical biopsy.

It will be apparent from the preceding description that methods provided by the present disclosure involve a degree of quantification to determine elevated or enhanced methylation of nucleic acid in tissue that is suspected of comprising a cancer cell or metastases thereof, or reduced gene expression in tissue that is suspected of comprising a cancer cell or metastases thereof. Such quantification is readily provided by the inclusion of appropriate control samples in the assays as described below.

As will be apparent to the skilled artisan, when internal controls are not included in each assay conducted, the control may be derived from an established data set.

Data pertaining to the control subjects are selected from the group consisting of:

1. a data set comprising measurements of the degree of methylation and/or gene expression for a typical population of subjects known to have a particular form of cancer that is currently being tested or a typical population of subjects known to have cancer generally;
2. a data set comprising measurements of the degree of methylation and/or gene expression for the subject being tested wherein said measurements have been made previously, such as, for example, when the subject was known to healthy or, in the case of a subject having cancer, when the subject was diagnosed or at an earlier stage in disease progression;
3. a data set comprising measurements of the degree of methylation and/or gene expression for a healthy individual or a population of healthy individuals;
4. a data set comprising measurements of the degree of methylation and/or gene expression for a normal individual or a population of normal individuals; and
5. a data set comprising measurements of the degree of methylation and/or gene expression from the subject being tested wherein the measurements are determined in a matched sample.

Those skilled in the art are readily capable of determining the baseline for comparison in any diagnostic assay of the present invention without undue experimentation, based upon the teaching provided herein.

In the present context, the term “typical population” with respect to subjects known to have cancer shall be taken to refer to a population or sample of subjects diagnosed with a specific form of cancer that is representative of the spectrum of subjects suffering from that cancer. Alternatively, a panel of subjects suffering from a variety of cancers (e.g., of the same tissue or cancer generally) that is representative of the spectrum of subjects suffering from cancer is used. This is not to be taken as requiring a strict normal distribution of morphological or clinicopathopathological parameters in the population, since some variation in such a distribution is permissible. Preferably, a “typical population” will exhibit a spectrum of cancers at different stages of disease progression and with tumors at different stages and having different morphologies or degrees of differentiation.

In the present context, the term “healthy individual” shall be taken to mean an individual who is known not to suffer from cancer, such knowledge being derived from clinical data on the individual. It is preferred that the healthy individual is asymptomatic with respect to the any symptoms associated with cancer.

The term “normal individual” shall be taken to mean an individual having a normal level of chromatin modification and/or gene expression as described herein in a particular sample derived from said individual.

As will be known to those skilled in the art, data obtained from a sufficiently large sample of the population will normalize, allowing the generation of a data set for determining the average level of a particular parameter. Accordingly, the level of chromatin modification and/or gene expression as described herein can be determined for any population of individuals, and for any sample derived from said individual, for subsequent comparison to levels determined for a sample being assayed. Where such nomialized data sets are relied upon, internal controls are preferably included in each assay conducted to control for variation.

The term “matched sample” shall be taken to mean that a control sample is derived from the same subject as the test sample is derived, at approximately the same point in time. In one example, the control sample shows little or no morphological and/or pathological indications of cancer. Matched samples are not applicable to blood-based or serum-based assays. Accordingly, it is preferable that the matched sample is from a region of the same tissue as the test sample, however does not appear to comprise a cancer cell. For example, the matched sample does not include malignant cells or exhibit any symptom of the disease. For example, the sample comprises less than about 20% malignant cells, such as less than about 10% malignant cells, for example less than about 5% malignant cells, e.g., less than about 1% malignant cells. Morphological and pathological indications of malignant cells are known in the art and/or described herein.

The relative modification of chromatin and/or expression of LOC134466 and (optionally) one or more additional markers compared to a control is indicative of cancer or the response to therapy or the progression or recurrence of disease or metastasis.

In one example, the level(s) of modification of chromatin and (optionally) expression of LOC134466 and/or one or more additional markers are subjected to multivariate analysis to create an algorithm which enables the determination of an index of probability of the presence or absence of cancer, or metastasis or progression of cancer or response to treatment. Hence, in one example, the present disclosure provides a rule based on the application of a comparison of levels of biomarkers to control samples. In another example, the rule is based on application of statistical and machine learning algorithms. Such an algorithm uses the relationships between biomarkers and disease status observed in training data (with known disease status) to infer relationships which are then used to predict the status of patients with unknown status. Practitioners skilled in the art of data analysis recognize that many different forms of inferring relationships in the training data may be used without materially changing the present invention.

The term “status” shall be taken to include whether or not a subject suffers from cancer (i.e., diagnostic status), whether or not a cancer has progressed, whether or not a cancer has metastasized, and/or whether or not a subject is responding to treatment for a cancer.

Analysis as described in the preceding paragraphs can also consider clinical parameters or traditional laboratory risk factors.

Information as discussed above can be combined and made more clinically useful through the use of various formulae, including statistical classification algorithms and others, combining and in many cases extending the performance characteristics of the combination beyond that of any individual data point. These specific combinations show an acceptable level of diagnostic accuracy, and, when sufficient information from multiple markers is combined in a trained formula, often reliably achieve a high level of diagnostic accuracy transportable from one population to another.

Several statistical and modeling algorithms known in the art can be used to both assist in biomarker selection choices and optimize the algorithms combining these choices. Statistical tools such as factor and cross-biomarker correlation/covariance analyses allow more rational approaches to panel construction. Mathematical clustering and classification tree showing the Euclidean standardized distance between the biomarkers can be advantageously used. Pathway informed seeding of such statistical classification techniques also may be employed, as may rational approaches based on the selection of individual biomarkers (e.g., LOC134466) based on their participation across in particular pathways or physiological functions or individual performance.

Ultimately, formulae such as statistical classification algorithms can be directly used to both select biomarkers and to generate and train the optimal formula necessary to combine the results from multiple biomarkers into a single index. Often techniques such as forward (from zero potential explanatory parameters) and backwards selection (from all available potential explanatory parameters) are used, and information criteria are used to quantify the tradeoff between the performance and diagnostic accuracy of the panel and the number of biomarkers used. The position of the individual biomarkers on a forward or backwards selected panel can be closely related to its provision of incremental information content for the algorithm, so the order of contribution is highly dependent on the other constituent biomarkers in the panel.

Any formula may be used to combine biomarker results into indices or indexes useful in the practice of the invention. As indicated herein, and without limitation, such indices may indicate, among the various other indications, the probability, likelihood, absolute or relative risk, time to or rate of disease, conversion from one to another disease states, or make predictions of future biomarker measurements of cancer. This may be for a specific time period or horizon, or for remaining lifetime risk, or simply be provided as an index relative to another reference subject population.

The actual model type or formula used may itself be selected from the field of potential models based on the performance and diagnostic accuracy characteristics of its results in a training population. The specifics of the formula itself may commonly be derived from biomarker results in the relevant training population. Amongst other uses, such formula may be intended to map the feature space derived from one or more biomarker inputs to a set of subject classes (e.g. useful in predicting class membership of subjects as normal, at risk for having cancer, recurrence or metastasis thereof or responding/not-responding to treatment), to derive an estimation of a probability function of risk using a Bayesian approach (e.g. the risk of cancer or a metastatic or recurrence event), or to estimate the class-conditional probabilities, then use Bayes' rule to produce the class probability function as in the previous case.

Following analysis and determination of an index of probability of the presence or absence of cancer, or metastasis or progression of cancer or response to treatment, the index can be transmitted or provided to a third party, e.g., a medical practitioner for assessment. The index may be used by the practitioner to assess whether or not additional diagnostic methods are required, e.g., biopsy and histological analysis and/or other assays, or a change in treatment or commencement of treatment.

### Monitoring the Progression of Cancer

As the level of a marker of cancer varies with the progression of cancer, the methods described herein are useful for monitoring the progression of cancer in a subject. In this regard, the term “determining the progression of cancer” includes determining the stage or grade of cancer. For example, the method comprises determining the level of modification of chromatin within LOC134466 or expression of LOC134466 in a sample from a subject and comparing said level to a level previously determined for the subject or a control sample. An enhanced level of modification and/or reduced level of expression in the sample compared to the previously obtained sample indicates that the disease has progressed, e.g., the disease may have progressed to a more advanced stage or may have advanced from pre-clinical to clinical. Comparison to a control sample from a subject having a specific stage or grade permits identification of the sage or grade of the cancer in the subject.

The present invention is also useful for determining the degree or risk of metastasis of cancer, for example, by determining the stage of cancer. For example, the present invention is useful for determining metastasis of a cancer to a tissue, such as, for example, a lymph node, bone or lung.

Clearly, the detection of one or more markers additional to LOC134466 is encompassed by this example of the invention.

Methods for detecting markers are described herein and are to be taken to apply mutatis mutandis to this example of the invention.

### Monitoring the Efficacy of Treatment

As the method of the invention is useful for monitoring or determining the progression (e.g., stage) of cancer, it is also useful for determining the efficacy of a therapy for said disease.

For example, a method described herein is used to determine the level of modification of chromatin within LOC134466 or expression of LOC134466 in sample from a subject receiving treatment for cancer. This level is then compared to, for example, a healthy or control subject. An enhanced level of modification and/or reduced level of expression in the test sample indicates that the subject is not responding to treatment. A similar level or lower level of modification or a similar level or higher level of expression in the test sample and a control sample indicates that the subject is responding to or has responded to treatment for said disease.

In another example, the control sample is derived from a subject suffering from cancer or from the subject prior to commencing treatment or from a point in time earlier in the treatment. In this respect, a reduced level of modification or an increased level of expression in the test sample compared to the control sample indicates that the subject is responding to or has responded to treatment. An enhanced or similar level of modification or a reduced level of expression in the test sample compared to the control sample indicates that the subject is not or has not responded to treatment.

### Determining the Time to an Event

The method of the present disclosure is also useful for determining, for example, the risk of an event occurring or the timing to an event occurring. For example, the present invention is useful for determining the risk of a patient dying early as a result of cancer or determining the risk of metastasis or the timing to metastasis.

Such methods are also applicable to determining, for example, the risk of or time to development of one or more of the following:

(i) onset of clinical cancer;
(ii) the progression of cancer from one stage to another; or
(iii) the likelihood of response of a subject to a therapeutic or prophylactic agent.

To determine the time to an event or the risk of an event, e.g., the time to death of a subject, the level of a marker of the invention is determined in a series of subjects for which survival data is known. A Cox Proportional Hazards model (see, e.g. Cox and Oakes (1984), Analysis of Survival Data, Chapman and Hall, London, N.Y.) is defined with time to death or early death as the dependent variable, and the level of the marker detected as the independent variable. The Cox model provides the relative risk (RR) of death for a unit change in the level of the marker. The subjects may then be partitioned into subgroups at any threshold value of the level of the marker (on the CT scale), where all subjects with levels above the threshold have higher risk, and all patients with levels below the threshold have lower risk of death or time to death, or vice versa, depending on whether the marker is an indicator of bad (RR>1.01) or good (RR<1.01) prognosis. Thus, any threshold value will define subgroups of patients with respectively increased or decreased risk.

The Cox proportional hazard model is the most general of the regression models because it is not based on any assumptions concerning the nature or shape of the underlying survival distribution. The model assumes that the underlying hazard rate (rather than survival time) is a function of the independent variables (covariates); no assumptions are made about the nature or shape of the hazard function.

In another embodiment, a Cox's Proportional Hazard Model with Time-Dependent Covariates is used to determine the time to or risk of an event in cancer based on a marker described herein in a sample from a subject. An assumption of the proportional hazard model is that the hazard function for an individual (i.e., observation in the analysis) depends on the values of the covariates and the value of the baseline hazard. Given two individuals with particular values for the covariates, the ratio of the estimated hazards over time will be constant.

Other methods for determining the time to or risk of an event will be apparent to the skilled artisan and include, for example, exponential regression, normal regression, log-normal regression or stratified analysis.

Using any of these forms of analysis a level of detection of a marker is determined that is predictive of the risk or time to an event. For example, a level of modification of a chromatin in LOC134466 in a sample is determined above which a subject is likely to live for fewer than a predetermined number of years. Alternatively, a subject with a lower level of the modification is likely to live for more than the predetermined number of years.

This form of analysis is useful for determining the risk of an event occurring in a subject or the time to an event occurring in a subject.

Accordingly, one example of the invention provides a method of detei wining a time to an event in cancer or the risk of an event occurring in cancer in a subject, the method comprising determining a the level of modified chromatin within a LOC134466 gene and/or determining the level of expression of a LOC134466 gene in a sample from the subject, wherein an enhanced level modification or reduced level of expression is indicative of the time to an event in the cancer.

### Methods of Treatment

The present invention additionally provides a method of treatment of cancer. Such a method comprises, for example diagnosing cancer using a method of the disclosure described in any one or more examples described herein and administering a suitable therapeutic and/or prophylactic compound or performing surgery or recommending treatment with a suitable therapeutic/prophylactic agent or recommending performance of surgery.

### Kits

The present disclosure additionally provides a kit for use in a method of the invention. In one embodiment, the kit comprises:
(i) one or more probes or primers (or isolated antibodies or ligands) that specifically hybridize to a marker described herein according to any example; and
(ii) detection means.

In another example, a kit additionally comprises a reference sample. Such a reference sample may for example, be a protein sample derived from a sample isolated from one or more subjects suffering from cancer. Alternatively, a reference sample may comprise a sample isolated from one or more normal healthy individuals.

In one example, the kit comprises a probe or primer. In one example, the probe or primer that is capable of selectively hybridizing to a gene or pseudogene described herein according to any example.

In those cases where the probe is not already available, they must be produced. Apparatus for such synthesis is presently available commercially, such as the Applied Biosystems 380A DNA synthesizer and techniques for synthesis of various nucleic acids are available in the literature. Methods for producing probes or primers are known in the art and/or described herein.

In one example, a probe or primer selectively hybridizes to a position in a LOC134466 gene that is selectively mutated by, for example, bisulphite treatment if the residue is not methylated. In another example, a probe or primer selectively hybridizes to a position in a LOC134466 gene comprising cytosine residues that can be methylated in a cancer cell.

The present invention also contemplates probes or primers that hybridize to a position in nucleic acids encoding one or more of ARMCX1, ICAM4, IL18, PEG3, PYCARD, SGNE1, ZNF177, wherein the position comprises cytosine residues that can be methylated in cancer. In one example, the probe or primer hybridizes to a sequence comprising the cytosine residue. In another example, the probe or primer binds to a sequence in which the cytosine residue is mutated, e.g., by treatment with bisulphite.

The kit may further comprise instructions for the detection of methylation levels of any of the target genes disclosed herein and for the comparison of those methylation levels with a reference level. The instructions may provide one or a series of cut-off values demarcating the likelihood of risk of a subject having, or being predisposed to cancer.

The present disclosure additionally provides a kit or an article of manufacture comprising a compound for therapeutic or prophylactic treatment of cancer packaged with instructions to perform a method substantially as described herein according to any example of the disclosure.

### Knowledge-Based Systems

Knowledge-based computer software and hardware for implementing an algorithm of the invention also form part of the present invention. Such computer software and/or hardware are useful for performing a method of the disclosure. Thus, the present disclosure also provides software or hardware programmed to implement an algorithm that processes data obtained by performing the method of the disclosure via an univariate or multivariate analysis to provide a disease index value and provide or permit a diagnosis of cancer and/or determine progression or status of a cancer or determine whether or not a cancer has progressed or determine whether or not a subject is responding to treatment for cancer in accordance with the results of the disease index value in comparison with predetermined values.

In one example, a method of the disclosure may be used in existing knowledge-based architecture or platforms associated with pathology services. For example, results from a method described herein are transmitted via a communications network (e.g. the interne) to a processing system in which an algorithm is stored and used to generate a predicted posterior probability value which translates to the index of disease probability or risk of recurrence or metastasis or responsiveness to treatment which is then forwarded to an end user in the form of a diagnostic or predictive report.

The method of the disclosure may, therefore, be in the form of a kit or computer-based system which comprises the reagents necessary to detect the concentration of the biomarkers and the computer hardware and/or software to facilitate determination and transmission of reports to a clinician.

The assay of the present invention permits integration into existing or newly developed pathology architecture or platform systems. For example, the present invention contemplates a method of allowing a user to determine the status of a subject with respect to a cancer, the method including:

(a) receiving data in the form of levels of modification of chromatin within a LOC134466 gene and/or expression of LOC134466 gene, optionally in combination with another marker of cancer;
(b) processing the subject data via univariate and/or multivariate analysis to provide a disease index value;
(c) determining the status of the subject in accordance with the results of the disease index value in comparison with predetermined values; and
(d) transferring an indication of the status of the subject to the user via the communications network reference to the multivariate analysis includes an algorithm which performs the multivariate analysis function.

In one example, the method additionally includes:

(a) having the user determine the data using a remote end station; and
(b) transferring the data from the end station to the base station via the communications network.

The base station can include first and second processing systems, in which case the method can include:

(a) transferring the data to the first processing system;
(b) transferring the data to the second processing system; and
(c) causing the first processing system to perform the univariate or multivariate analysis function to generate the disease index value.

The method may also include:

(a) transferring the results of the univariate or multivariate analysis function to the first processing system; and
(b) causing the first processing system to determine the status of the subject.

In this case, the method also includes at least one of:

(a) transferring the data between the communications network and the first processing system through a first firewall; and
(b) transferring the data between the first and the second processing systems through a second firewall.

The second processing system may be coupled to a database adapted to store predetermined data and/or the univariate or multivariate analysis function, the method include: (a) querying the database to obtain at least selected predetermined data or access to the multivariate analysis function from the database; and

(b) comparing the selected predetermined data to the subject data or generating a predicted probability index.

The second processing system can be coupled to a database, the method including storing the data in the database.

The method can also include having the user determine the data using a secure array, the secure array of elements capable of determining the level of biomarker and having a number of features each located at respective position(s) on the respective code. In this case, the method typically includes causing the base station to:

(a) determine the code from the data;
(b) determine a layout indicating the position of each feature on the array; and
(c) determine the parameter values in accordance with the determined layout, and the data.

The method can also include causing the base station to:

(a) determine payment information, the payment information representing the provision of payment by the user; and
(b) perform the comparison in response to the determination of the payment information.

The present invention also provides a base station for determining the status of a subject with respect to a cancer, the base station including:

(a) a store method;
(b) a processing system, the processing system being adapted to:
(i) receive subject data from the user via a communications network;
(iii) determining the status of the subject in accordance with the results of the algorithmic function including the comparison; and
(c) output an indication of the status of the subject to the user via the communications network.

The processing system can be adapted to receive data from a remote end station adapted to determine the data.

The processing system may include:

(a) a first processing system adapted to:
(i) receive the data; and
(ii) determine the status of the subject in accordance with the results of the univariate or multivariate analysis function including comparing the data; and
(b) a second processing system adapted to:
(i) receive the data from the processing system;
(ii) perform the univariate or multivariate analysis function including the comparison; and
(iii) transfer the results to the first processing system.
The base station typically includes:
(a) a first firewall for coupling the first processing system to the communications network; and
(b) a second firewall for coupling the first and the second processing systems.

The processing system can be coupled to a database, the processing system being adapted to store the data in the database.

The present disclosure is now described further in the following non-limiting examples.

### EXAMPLES

### Example 1

### Markers of Breast Cancer

### 1.1 Methods

### Cell Line, Tissue and OSE Collection and Processing

Eight epithelial ovarian cancer (EOC) and 2 immortalized ovarian surface epithelium (HOSE) cell lines (Table 1) were collected and cultured essentially as described previously (Barton et al., Br J Cancer 102(1): 87-96, 2009). Cell lines were authenticated by short tandem repeat polymorphism, single nucleotide polymorphism, and fingerprint analyses and passaged for less than 6 months. Forty-seven fresh frozen tumor tissue samples were obtained with informed consent from women undergoing debulking surgery for EOC at the Royal Hospital for Women (RHW, Sydney Australia), snap frozen in liquid nitrogen and stored at −80° C. One hundred archival formalin-fixed paraffin embedded (FFPE) tumor samples were processed at the RHW and blocks were acquired for DNA methylation analysis. A section from each tumor sample was stained with Haematoxylin and Eosin and regions containing >80% tumor were marked and the corresponding piece removed for DNA extraction. Seventeen OSE were obtained with consent, by scraping the ovary during surgery for non-ovarian gynaecological malignancies followed by establishment of epithelial cells in culture. Cultures were evaluated for purity by staining for high molecular weight cytokeratin to exclude stromal contamination and maintained in culture essentially as previously described (Rosen et al., Methods Enzymol. 2006; 407:660-676, 2006). Cell pellets from passage three or less were processed for DNA.

### Nucleic Acid Extraction and Processing

Total RNA for RT-PCR was extracted with Qiagen RNeasy mini kit (Qiagen, Almeda Calif., USA). 1 μg total RNA for RT-PCR was DNAse treated (Ambion, Austin Tex., USA) and reverse transcribed using oligo dT primers (Promega, Alexandria NSW Australia). Genomic DNA was extracted from tumor and OSE with Qiagen QiAMP mini kits (Qiagen, Almeda Calif., USA) and from cell lines with the Stratagene DNA extraction Kit (Agilent, Santa Clara Calif., USA). 1-2 μg genomic control DNA (Roche Applied Sciences, Indianapolis USA) in vitro methylated DNA (Chemicon International, Temecula Calif., USA) and RNase treated cell line & tumor DNA was bisulphite converted either using the Epitect kit (Qiagen, Almeda Calif., USA) or essentially as previously described (Clark et al., Nat. Protoc. 1(5): 2353-2364, 2006; Clark et al., Nucleic Acids Res. 22(1.5):2990-2997, 1994).

### Pharmacological Reactivation of Methylated Genes

Experiments were performed in triplicate using ovarian cancer cell lines A2780 and CaOV3, derived from primary Type II EOC. Cell lines were treated at 30% confluence with 5 μM and 2.5 μMrespectively (concentrations previously optimized to minimize cellular toxicity, data not shown), with the DNA methyltransferase inhibitor 5-aza-2′ deoxycytidine (5-Aza-dC), (Sigma Aldrich, St. Louis Mo., USA) for 24 hours. Cells were then expanded to 90% confluency, media changed every 24 hr before extracting RNA (RNeasy, Qiagen). As a positive control, re-expression and DNA demethylation for the methylated gene DLEC1 was verified by qPCR and clonal bisulphite sequencing.

### DNA Methylation Analysis

Sequenom massARRAY Quantitative Methylation Analysis

T7 tagged Sequenom methylation PCR assays were designed essentially as described in (Coolen, et al., Nucleic Acids Res 35: e119) for bisulphite converted DNA specificity, and tested for bias using a thermal gradient on mixes of 50% methylated:unmethylated template. Assays were performed in triplicate as per conditions indicated in Table 2 and SYBR heat dissociation curves using ABI 7900HT to ensure appropriate amplification. Replicates were pooled, treated with shrimp alkaline phosphatase (SAP), reverse transcribed, cleaved and applied to spectrochips essentially according to manufacturer's instructions for MALDI-TOF analysis (Sequenom, San Diego Calif., USA) and results analyzed using epityper software, the R (R_DevelopmentCoreTeam, 2010) package RseqMeth (Statham, et al., Bioinformatics 26: 1662-1663, 2010) and Microsoft Excel™. CpG methylation levels were averaged across the amplicon and average methylation levels greater than 25% were called positive.

Clonal bisulphite sequencing analysis was performed on selected pooled Sequenom PCR products, essentially as previously described (Clark, et al., Nucleic Acids Res 22: 2990-2997, 1994).

### Methylation Specific Headloop Suppression PCR Assay (MSH-PCR)

Methylation specific headloop suppression assay (MSH-PCR) was designed essentially as previously described (Devaney, et al., Cancer Epidemiol Biomarkers Prey. 2010), with MSH-PCR directed against CpG methylation at the transcriptional start site (TSS) of LOC134466. MSH-PCR reaction conditions were optimized to distinguish methylated from unmethylated DNA using fully methylated (Chemicon International, Temecula Calif., USA) and unmethylated control (OV90) DNA. Triplicate MSH-PCRs were performed on bisulphite converted DNA from 100 FFPE EOC and 15 EOC. The melting temperature (Tm) of the amplicon was calculated from the derivative SYBR signal during a heat dissociation cycle. Samples were considered methylated with a Tm of >78° C. as compared to the fully methylated control DNA.

### Expression Arrays and Analysis

Total RNA from A2780 and CaOV3 cell lines, plus and minus treatment with 5-Aza-dC was characterized on the Agilent bioanalyzer RNA nano chip (Agilent, Foster City Calif., USA) for an RNA integrity number (RIN) of >9.0, labeled and hybridized to Affymetrix human genome U133 plus 2 GeneChips essentially according to manufacturer's instructions. The GeneChip scans were analyzed by GCOS (Affymetrix) and resulting probeset intensities normalized by the robust multi-array average (RMA) method (Irizarry, et al., Biostatistics 4: 249-264, 2003). Principle components analysis (PCA) indicated that the two cell lines' expression profiles were distinct (data not shown). The data were normalized separately for each cell line and robust multichip average (RMA) values for analysis were imported into Genespring GX 7.3. Genes re-expressed by 5-Aza-dC were identified as “absent” (undetectable) in all three replicate untreated samples and “present” or “marginal” (detectable) in two of three replicate 5-Aza-dC treated samples. Unsupervised hierarchical clustering was used to show that the array expression results for genes identified as of interest were capable of distinguishing 5-Aza-dC treated from untreated cell lines.

Quantitative RT-PCR (qRT-PCR) of mRNA levels was used to verify re-expression of five genes (ARMCX1, ICAM4, KLF4, PEG3, PYCARD). qRT-PCR was performed in triplicate using the ABI 7900HT Real-Time PCR System (Applied Biosystems, Foster City Calif., USA) under standard thermocycling with ABI Power SYBR mastermix (Applied Biosystems), diluted cDNA. Primer sequences are shown in Table 2.

### DNA Methylation Profiling and Analysis

Whole genome DNA methylation profiling was performed on EOC cell lines A2780 & CaOV3 (in duplicate) from three normal ovarian surface epithelium samples, essentially as previously described (Coolen et al., Nat Cell Biol. 12: 235-246 2010). Briefly, 1 microgram genomic DNA was fragmented to a mean fragment length of ˜400 bp and Immunoprecipitation (IP) of methylated DNA was performed using a mouse anti-5-methylcytosine MAb (Millipore, Billerica Mass., USA) and enriching on protein A/G agarose beads (Santa Cruz Biotechnology, Santa Cruz Calif., USA). IP and total genomic DNA were purified by phenol:chloroform extraction and subjected to whole genome amplification (WGA) in duplicate (WGA2+, Sigma Aldrich, St. Louis Mo., USA). SYBR qPCR was performed to ensure the ratio of enrichment of methylated DNA in the IP to input was retained through WGA before amplified samples were fragmented, labeled and applied to Affymetrix whole genome promoter 1.0R tiling arrays and scanned. Array CEL files were normalized and analyzed using MAT (Johnson et al., Proc Natl Acad Sci USA. 103: 12457-12462, 2006) for aroma.affymetrix (Bengtsson et al., Tech Report #745, Department of Statistics, University of California, Berkeley, February 2008) with a smoothing window of 600 bp. Output was visualized in the integrated genome browser (IGB, Affymetrix) and interrogated using the Repitools package (Statham, et al., Bioinformatics 26: 1662-3 2010). The Repitools function ‘significancePlot’ was used to visualize averaged promoter methylation of multiple genes, whereby normalized MeDIP-chip output at a selection of gene promoters was averaged and compared to the output of 1000 random gene selections of the same size. To assess the extent of copy number aberrations in the samples, promoter tiling profiles of total DNA inputs were compared to a genomic reference. Large scale copy number variations (CNV) were observed in the cancer cell lines, particularly the P53 mutant CaOV3, consistent with SNP data from the cancer genome project, whereas no large scale CNV were observed in OSE. To address the potential impact of copy number differences on readout, all array data was normalized to inputs before comparisons were made between samples.

### Candidate Selection

Genes were selected for further evaluation if they met three of the following criteria: 1) re-expressed by 5-Aza-dC; 2) contained a CpG island associated promoter, as determined by the USCS genome browser (human genome build 18); 3) down-regulated in ovarian cancer, according to the Bonome et al 2005 study that compared 54 type II EOC and 10 OSE (Bonome, et al., Cancer Res 65: 10602-10612, 2005). A potential role in cancer was ascertained from the NCBI entrez gene reference into function and PubMeth.

TABLE 1
Cell lines used in analysis

| Line | Type | Culture Medium/% FBS | Source |
|---|---|---|---|
| A2780 | Serous Ovarian Adenocarcinoma | RPMI/10 | Tumor Institute Milan Italy |
| IGROV1 | Adenocarcinoma | RPMI/10 | Tumor Institute Milan Italy |
| OV90 | Serous Ovarian Adenocarcinoma | DMEM/10 | ATCC |
| CAOV3 | Serous Ovarian Adenocarcinoma | DMEM/10 | ATCC |
| TOV112D | Endometrioid Ovarian Adenocarcinoma | DMEM/15 | ATCC |
| TOV21G | Clear Cell Ovarian Adenocarcinoma | DMEM/15 | ATCC |
| SKOV3 | Adenocarcinoma | RPMI/10 | ATCC |
| OVCAR3 | Adenocarcinoma | RPMI/10 | ATCC |
| COLO316 | Serous Ovarian Adenocarcinoma | RPMI/10 | Department of Gynaecological Oncology |
| | | | Cell Bank, Westmead Australia |
| EFO27 | Mucinous | RPMI + pyruvate/20 | Viola |
| HOSE 6.3 | Transformed Ovarian Surface Epithelium | M199:MCDB105/10 | University of Toyko |
| HOSE 17.1 | Transformed Ovarian Surface Epithelium | M199:MCDB105/10 | Department of Gynaecological Oncology |
| | | | Cell Bank, Westmead Australia |

| | | | | | | Opt |
|---|---|---|---|---|---|---|
| | | | | | | Annealing |
| | Gene Name | Forward Primer | | Reverse Primer | | temperature |
| BSP-T7 Tagged | ARMXC1 | AGGAAGAGAGTGGTAAAGAGGAA | SEQ ID NO: 44 | CAGTAATACGACTCACTATAGGGAGAAGG | SEQ ID NO: 45 | 63.8 |
| Thermocycling | | AATGAGTGATAGGATT | | CTCCACACACAAACCTACTAAAATCCAAA | | |
| 95′ 5 min | BMP6 | AGGAAGAGAGGTAATGGGGTAAA | SEQ ID NO: 46 | CAGTAATACGACTCACTATAGGGAGAAGG | SEQ ID NO: 47 | 63.8 |
| 5 cycles | | TTTTATGGTGGTT | | CTRAACTACACTACAACCTACRCCCTAA | | |
| 95′ 30 sec | CST6 | AGGAAGAGAGTTTTTTYGTGAAT | SEQ ID NO: 48 | CAGTAATACGACTCACTATAGGGAGAAGG | SEG ID NO: 49 | 60.6 |
| Opt Ta′ 90 sec | | YGTTTTTGTATTGGTAT | | CTCCRTCRAAACCCTCAAAACCRTAAATA | | |
| 72′ 90 sec | HSPA2 | AGGAAGAGAGTAGGAATTGAGYG | SEQ ID NO: 50 | CAGTAATACGACTCACTATAGGGAGAAGG | SEQ ID NO: 51 | 63.8 |
| 45 cycles | | AATTTTTTTAGTGGTTT | | CTCCAACTCCACCACAAACTTACCTAAA | | |
| 95′ 30 sec | ICAM4 | AGGAAGAGAGGGGAGYGGAATTT | SEQ ID NO: 52 | CAGTAATACGACTCACTATAGGGAGAAGG | SEQ ID NO: 53 | 62 |
| 65′ 90 sec | | TTTTTTGTAGTATTT | | CTAAAAACRCRAAAAAACTACCCTTAAA | | |
| 72′ 90 sec | IL18 | AGGAAGAGAGAGAGGTATAGGTT | SEQ ID NO: 54 | CAGTAATACGACTCACTATAGGGAGAAGG | SEQ ID NO: 55 | 60.6 |
| | | TTGGAAGGTATAGAGTT | | CTTCTTCCCRAAACTATATAAACTACAAC | | |
| | | | | AAA | | |
| | KLF4 | AGGAAGAGAGGATTTATTAGTTT | SEQ ID NO: 56 | CAGTAATACGACTCACTATAGGGAGAAGG | SEQ ID NO: 57 | 60.5 |
| | | TYGYGGGTTTYGAATT | | CTCCRAAACTCCCTTCCATCRTTACTATA | | |
| | | | | A | | |
| | LOC134466:RC | AGGAAGAGAGGTGAAATTTGTTT | SEQ ID NO: 58 | CAGTAATACGACTCACTATAGGGAGAAGG | SEQ ID NO: 59 | 66.8 |
| | | GTAAAAGTGTGGATT | | CTAATAAAAACCRCTATCACATCCATATT | | |
| | | | | AA | | |
| | LTBP2:RC | AGGAAGAGAGGTTTTTTAGATTT | SEQ ID NO: 60 | CAGTAATACGACTCACTATAGGGAGAAGG | SEQ ID NO: 61 | 63.8 |
| | | AGAGAAGTTGG | | CTTCCRAAACAAACTAAACCTACTAAA | | |
| | PAPLN | AGGAAGAGAGTYGGAGTAGTTTY | SEQ ID NO: 62 | CAGTAATACGACTCACTATAGGGAGAAGG | SEQ ID NO: 63 | 60.6 |
| | | GTGATTAGATT | | CTCCTACTAAAACCAAACTAAACCTACTA | | |
| | | | | AA | | |
| | PARVA | AGGAAGAGAGATAGTGYGATTTA | SEQ ID NO: 64 | CAGTAATACGACTCACTATAGGGAGAAGG | SEQ ID NO: 65 | 55.7 |
| | | GTAGGTTTTGT | | CTACCTAAATTCACAACAAACCCATA | | |
| | PEG3 long | AGGAAGAGAGTGAGGTTGTTGAT | SEQ ID NO: 66 | CAGTAATACGACTCACTATAGGGAGAAGG | SEQ ID NO: 67 | 63.8 |
| | | TGGTTAGTATAGGAAGTT | | CTACRCACTCACCTCACCTCAATACTA | | |
| | PEG3 short | AGGAAGAGAGYGTTAAATTGTTG | SEQ ID NO: 68 | CAGTAATACGACTCACTATAGGGAGAAGG | SEQ ID NO: 69 | 62 |
| | | TTGTGGTAATYGTAGTT | | CTTATACCCACTCTCRAACTAAACAACRA | | |
| | | | | A | | |
| | PTGER3 | AGGAAGAGAGGATATTTTAGYGG | SEQ ID NO: 70 | CAGTAATACGACTCACTATAGGGAGAAGG | SEQ ID NO: 71 | 63.8 |
| | | AGAATTGGAGGAGATT | | CTCRACTAAAACTAAACTACCCCCCATAA | | |
| | | | | TA | | |
| | PYCARD | AGGAAGAGAGAGAAATTYGAGGT | SEQ ID NO: 72 | CAGTAATACGACTCACTATAGGGAGAAGG | SEQ ID NO: 73 | 63.8 |
| | | TTTAAGTTTAGGTGTTT | | CTAAAAACRCTTCCTTACTACACCCTTAA | | |
| | SCIN | AGGAAGAGAGGGTAGTGATGGGT | SEQ ID NO: 74 | CAGTAATACGACTCACTATAGGGAGAAGG | SEQ ID NO: 75 | 63.8 |
| | | ATGTATAAAGGAGTGTT | | CTACCTTATTCRCCRCCACTTTATAATA | | |
| | SGNE1 | AGGAAGAGAGTTGTGYGGTTTTT | SEQ ID NO: 76 | CAGTAATACGACTCACTATAGGGAGAAGG | SEQ ID NO: 77 | 60.6 |
| | | GGTGAGAGGTATA | | CTAACCTCCACCTCAAAAATTTTAACAAA | | |
| | SOCS1 | AGGAAGAGAGAGATTAGGYGYGG | SEQ ID NO: 78 | CAGTAATACGACTCACTATAGGGAGAAGG | SEQ ID NO: 79 | 63.8 |
| | | ATTTTAGTTTGGTT | | CTTAAAACRCTACRAAACCRAACAAATAT | | |
| | | | | A | | |
| | SPOCK2:RC | AGGAAGAGAGGYGGGAGGAGAGT | SEQ ID NO: 80 | CAGTAATACGACTCACTATAGGGAGAAGG | SEG ID NO: 81 | 63.8 |
| | | TGAGGATAGT | | CTAACCCCRAATCTCTACCTTAACAA | | |
| | TLE4 | AGGAAGAGAGGTTATTTGTTAGT | SEQ ID NO: 82 | CAGTAATACGACTCACTATAGGGAGAAGG | SEQ ID NO: 83 | 60.6 |
| | | AGTGAAATGATGGGTAT | | CTACACAACTCTAACAACACRCTCTATAT | | |
| | | | | AAA | | |
| | ZNF177 | AGGAAGAGAGGAATTYGTTAAAT | SEQ ID NO: 84 | CAGTAATACGACTCACTATAGGGAGAAGG | SEQ ID NO: 85 | 66.8 |
| | | GTGYGAGTTGGGTAGTT | | CTAAATAACAACCCCAAACCCAACAATA | | |
| | ZNF542 | AGGAAGAGAGTTTGTGTTTTAGG | SEQ ID NO: 86 | CAGTAATACGACTCACTATAGGGAGAAGG | SEQ ID NO: 87 | 63.8 |
| | | TAGTTYGTTTTTTAGTT | | CTCCTAAACRAATCCCTAAACCTCTAA | | |
| SYBR | ARMCX1 | TTGGAGCAGGAAGAAACGCA | SEQ ID NO: 88 | AACCCGAGCACAAAACAGGC | SEQ ID NO: 89 | |
| Expression | HSPA2 | AAGAGGCTGATTGGACGGAAAT | SEQ ID NO: 90 | TCTACTTGCACTTTGGGCTTGC | SEQ ID NO: 91 | |
| Thermocycling | ICAM4 | AAGGCAAGACGCTCAGAGGG | SEQ ID NO: 92 | GAGGCTCCAAAATCACGCTG | SEQ ID NO: 93 | |
| 95′ 10 min | IL18 | AACCTCAGACCTTCCAGATCGCT | SEQ ID NO: 94 | AGAGGCCGATTTCCTTGGTCA | SEQ ID NO: 95 | |
| 50′ 5 min | LOC13446 | CGGTGATAGGATTTTTGGGACC | SEQ ID NO: 96 | TGAAATCCACAGCCACATCCTC | SEQ ID NO: 97 | |
| 40 cycles | PEG3 | AGGGCTTATGAGTCCCGATCTC | SEQ ID NO: 98 | TCGACTGGTGCTTGGGTAGG | SEQ ID NO: 99 | |
| 95′ 30 sec | PYCARD | GCACTTTATAGACCAGCACCGG | SEQ ID NO: 100 | TGGTACTGCTCATCCGTCAGG | SEQ ID NO: 101 | |
| 60′ 90 sec | SCIN | TCTTACGAACGACCTGACAGCC | SEQ ID NO: 102 | ACGAGGAACCACACCACTGATAAA | SEQ ID NO: 103 | |
| Dissociation | TLE4 | CCCCGATTAGTCCAGCCTCT | SEQ ID NO: 104 | TGGATATGGACAAGGTACTGCCA | SEQ 10 NO: 105 | |
| ramp | ZNF177 | AGTAAGCAAGAAGAGGGCCTGG | SEQ ID NO: 106 | ACCGCATCTCTTTGGGTACTAGAA | SEQ ID NO: 107 | |

### 1.2 Results

### Discovery Pipeline of Methylated Genes in EOC.

Three steps were implemented to identify aberrantly silenced and methylated genes in EOC. First, to identify genes potentially silenced by DNA methylation, genome wide epigenetic reexpression profiles were generated for two ovarian cancer cell lines. A2780 and CaOV3 cells were treated with DNA methyltransferase inhibitor 5-Aza-deoxycytidine (5-Aza-dC), under conditions that induce re-expression and demethylation of the methylated gene DLEC1 and mRNA expression profiles were generated using Affymetrix whole genome HGU133 plus 2.0. Genes showing potential methylation-based silencing were identified if they were undetectable in the untreated cells, but re-expressed by 5-Aza-dC treatments. Using these criteria 947 and 1091 probesets were identified in A2780 and CaOV3 cells respectively, that were reactivated.

Second, to identify potentially methylated genes that were also down-regulated in clinical EOC, publicly available gene annotations and mRNA expression profiles were analyzed in 54 Type II EOC compared with 10 OSE tissue samples (>1.5 negative fold change, corrected p-value >0.05) (Bonome, et al., supra). Genes corresponding to the 5-Aza-dC re-expressed probesets were filtered based on alignment to a promoter associated CpG island (Gardiner-Garden and Frommer, J Mol. Biol., 196:261-282, 1987). The resulting gene lists were then compared with probesets consistently down-regulated in type II EOC, in order to enrich for genes likely to have a function in vivo. Genes displaying multiple criteria (i.e. reexpressed by 5-Aza-dC in either cell line and down regulated in EOC and/or associated with CpG islands) formed the basis for selection of candidates for further validation.

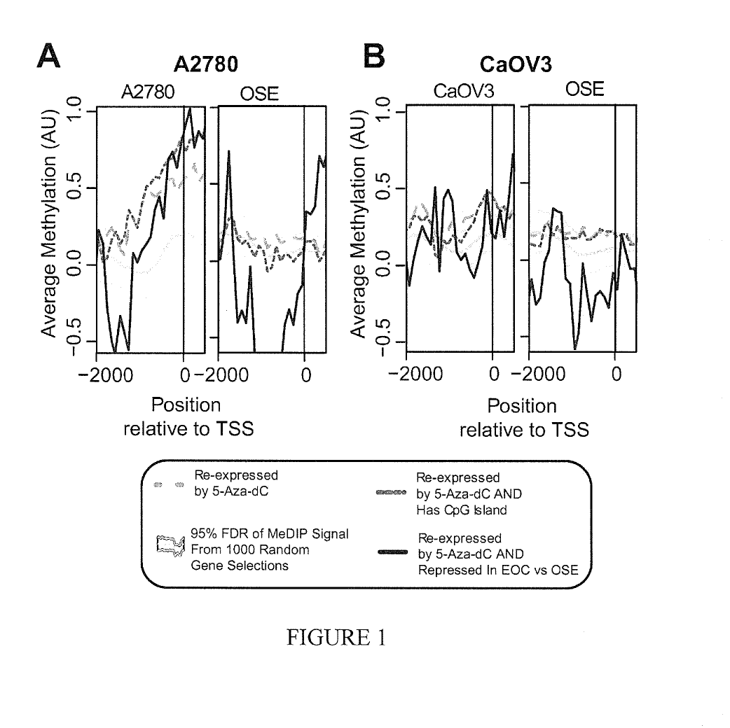

Third, genome-wide methylation profiles were generated by methylated DNA immunoprecipitation, followed by promoter tiling array analysis (MeDIP-Chip) for A2780, CaOV3 and three normal OSE samples. Direct measurement of DNA methylation was performed on candidate genes, by MeDIP-chip and Sequenom massARRAY gene promoters, to assess differential levels of DNA methylation. Averaged promoter methylation levels were plotted (−2000 bp to +500 bp relative to the transcription start site (TSS)) of the candidate gene list versus random gene selections in the cancer cell lines and compared to pooled OSE (FIG. 1). Both ovarian cancer cell lines showed an increase in methylation at the TSS compared to OSE, consistent with CpG island methylation being a hallmark of cancer (Baylin, AIDS Res Hum Retroviruses 8: 811-820, 1992). A2780 cells displayed hypermethylation at the TSS in all genes re-expressed by 5-Aza-dC, and a modest increase when limited to genes with CpG islands (FIG. 1 Panel A). Interestingly in CaOV3, minimal hypermethylation was observed for 5-Aza-dC responsive genes (FIG. 1 Panel B), suggesting that fewer genes are hypermethylated in CaOV3 relative to A2780.

### Validation of Candidate Gene Methylation and Expression in Multiple EOC Cell Lines

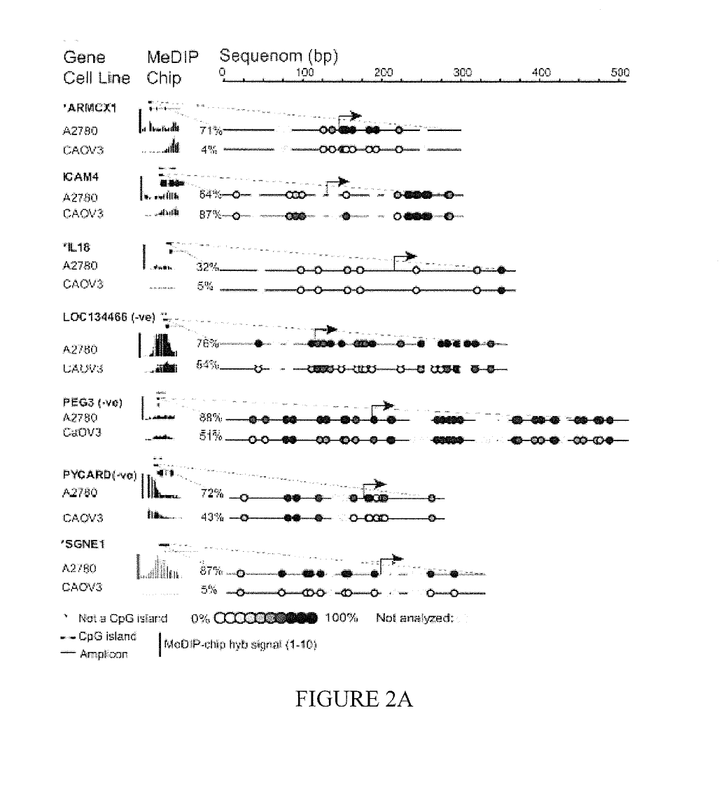

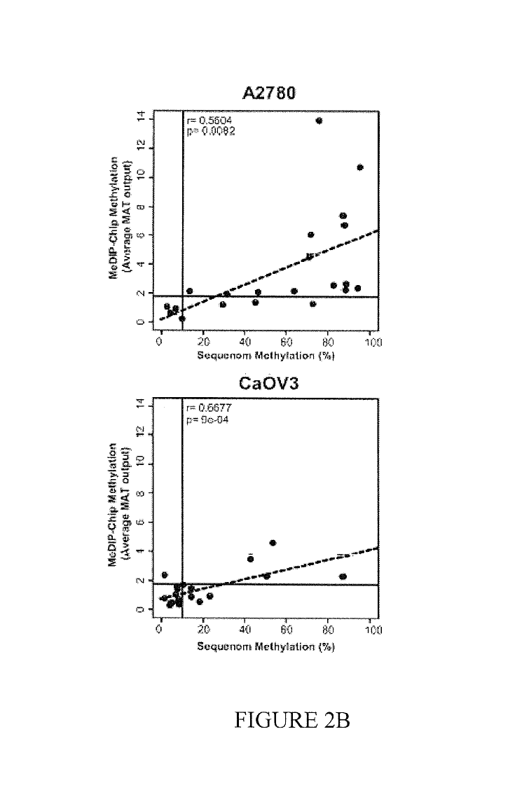

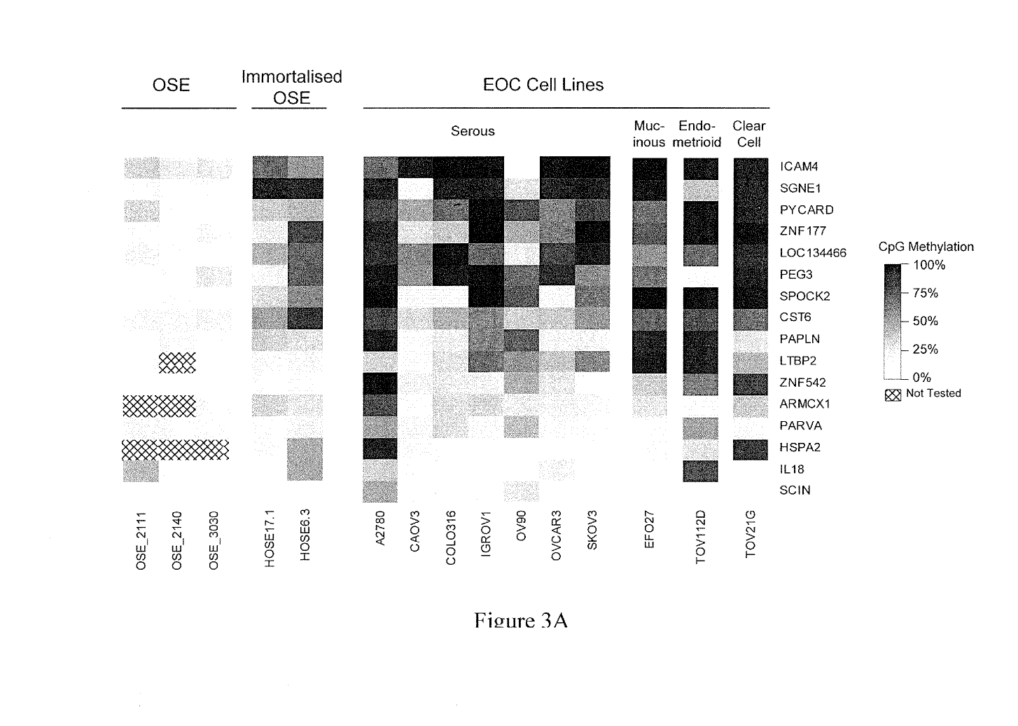

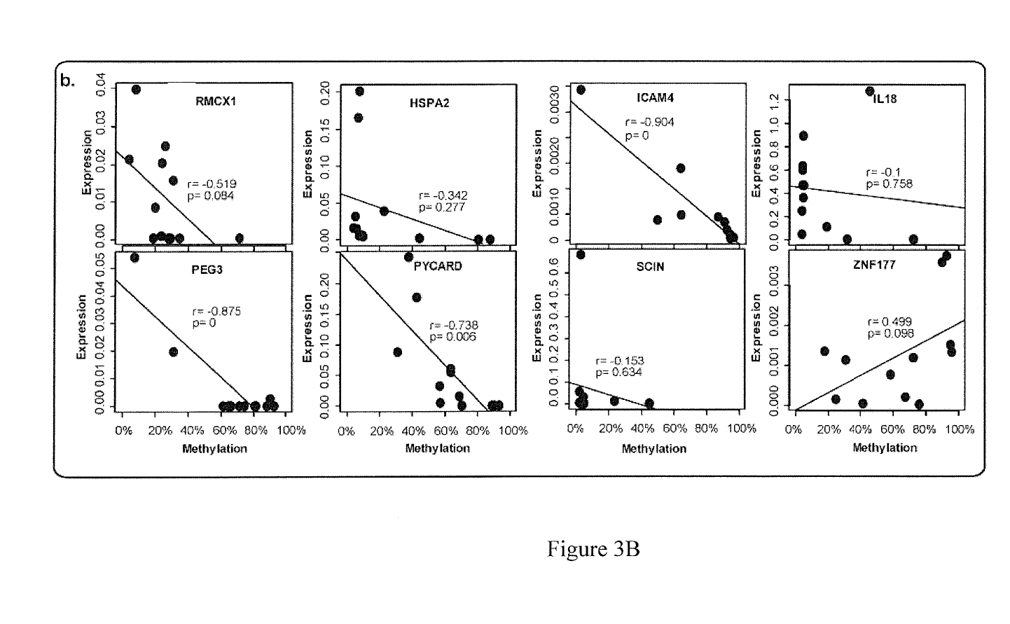

Twenty-one genes were chosen from the pool of 5-Aza-dC re-expressed genes, based on evidence of DNA methylation by MeDIP-chip and/or reported tumor suppressor function (Table 3), for further validation using Sequenom massARRAY assays on A2780 and CaOV3 DNA. Methylation between MeDIP-Chip and Sequenom assays were compared on a gene-by-gene basis and mapped relative to the TSS, with seven examples shown in FIG. 2A. The correlation between the two promoter methylation assays; MeDIP-chip and Sequenom was assessed for all 21 genes in both cell lines and was found to be 0.5604 and 0.6677 respectively (FIG. 2B). Sequenom assays validated that 16/21 (76%) genes were methylated in at least one of the ovarian cancer cell lines. Methylation frequency of these 16 genes was then tested in three OSE, two immortalized OSE cell lines (HOSE17.1 and HOSE6.3) and multiple EOC subtypes, including serous (IGROV1, OV90, SKOV3, OVCAR3 & COLO316), mucinous (EFO27), endometrioid (TOV112D) and clear cell (TOV21G) subtypes (FIG. 3A). 15/16 genes showed hypermethylation in the broad panel of EOC subtypes and a lack of methylation in normal OSE cells. SCIN1 was methylated in only one cancer cell line and was omitted from further analysis. To investigate if DNA methylation was associated with gene repression, eight genes that displayed some methylation in the cell lines were tested by qPCR in two immortalized OSE and eight EOC cell lines. Hypermethylation was associated with gene repression for seven (ARMCX1, HSPA2, IAMC4, IL18, PEG3, PYCARD and SCIN) of the eight genes tested (FIG. 3B), indicating a strong association between DNA methylation and gene inactivation.

### Gene Methylation in Primary Serous Ovarian Cancer and Normal Tissues

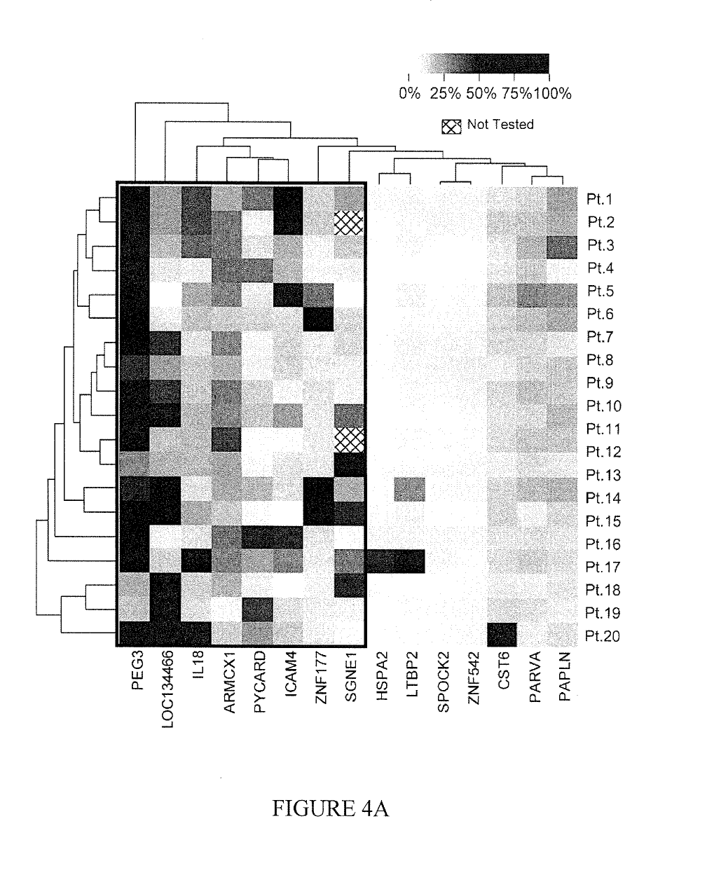

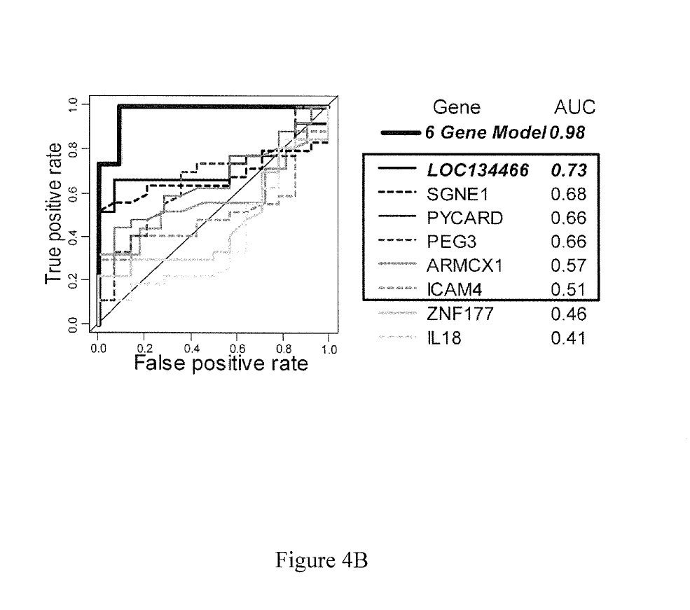

Next promoter methylation of the 15 genes was measured using Sequenom MassARRAY in a panel of 20 serous EOC patient samples (FIG. 4A). Hierarchical clustering by DNA methylation levels revealed that eight of these genes (ARMCX1, ICAM4, IL18, LOC134466, PEG3, PYCARD, SGNE1 and ZNF177) were hypermethylated in more than one tumor (FIG. 4A). Promoter methylation levels for these eight genes were then compared between cancer tissue (n=25-27) and OSE (n=14) by plotting a receiver operating characteristic (ROC), and calculating the area under the curve (AUC) of methylation levels in cancer versus normal (FIG. 4B). To evaluate the performance of the eight genes as a panel, a logistic regression model was fitted to the gene methylation data. Analysis of variance (ANOVA) revealed that a panel of 6 genes (ARMCX1, ICAM4, LOC134466, PEG3, PYCARD and SGNE1) was a potent discriminator of cancer versus normal, with a high AUC (0.98). In addition, methylation of LOC134466, was identified as the best individual discriminator (AUC=0.73), indicating that this putative gene may be a potential independent biomarker of EOC.

### LOC134466 is Commonly Hypermethylated in EOC

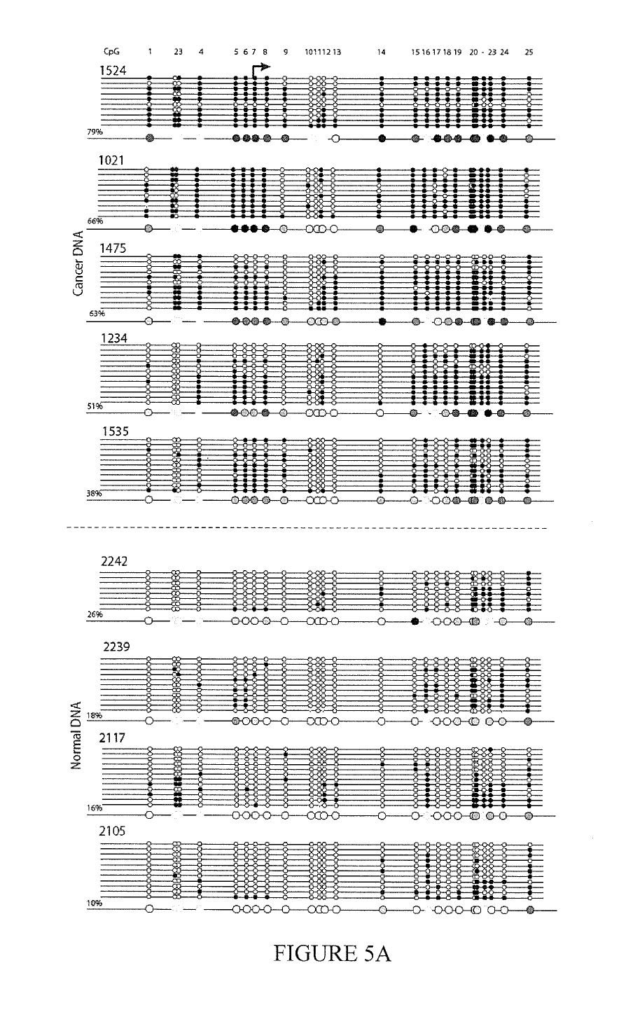

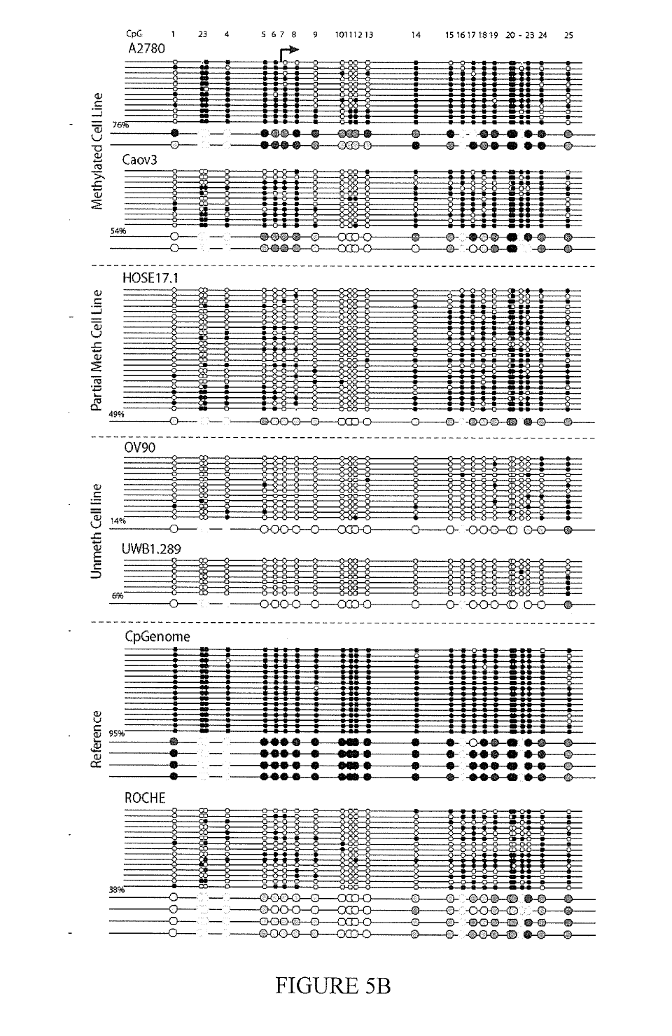

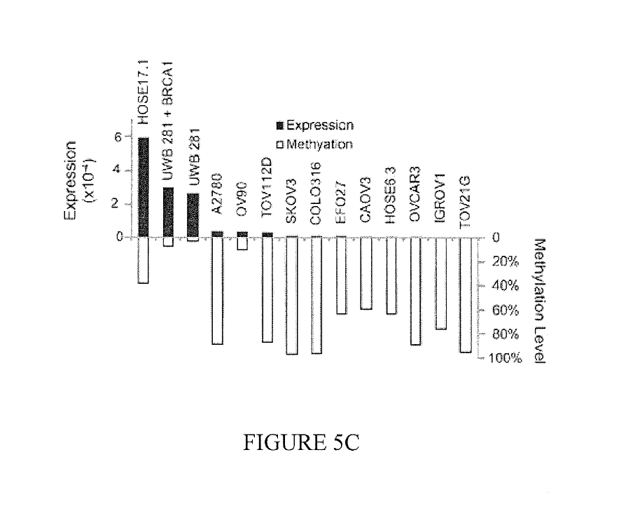

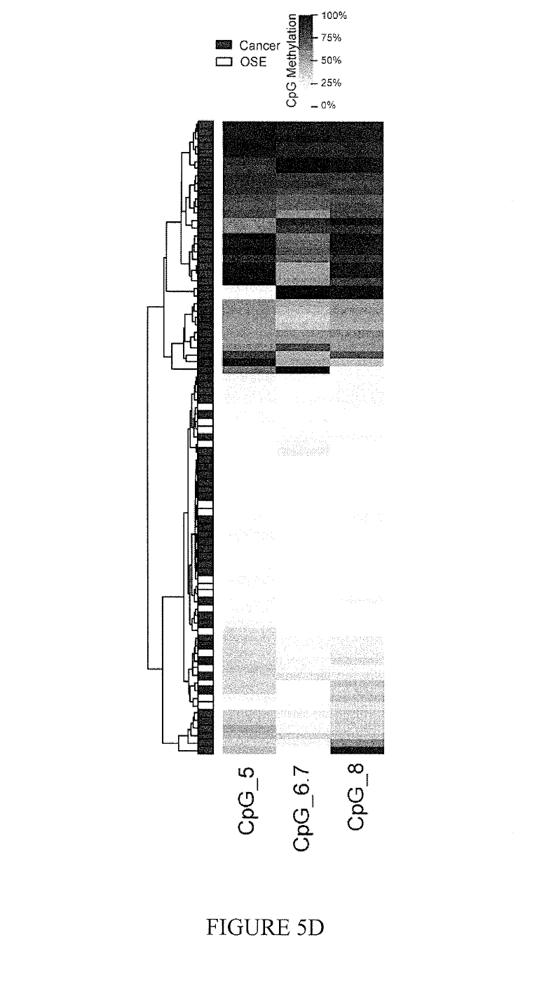

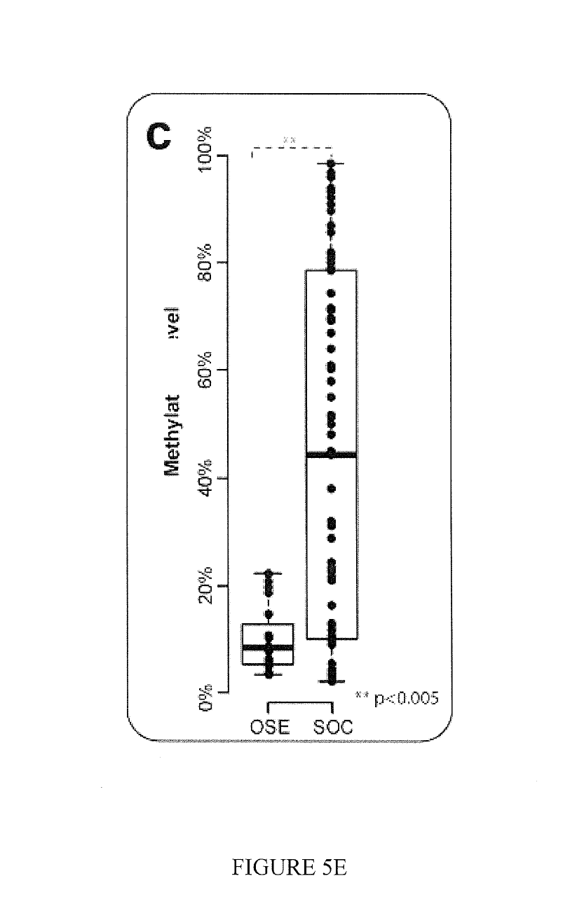

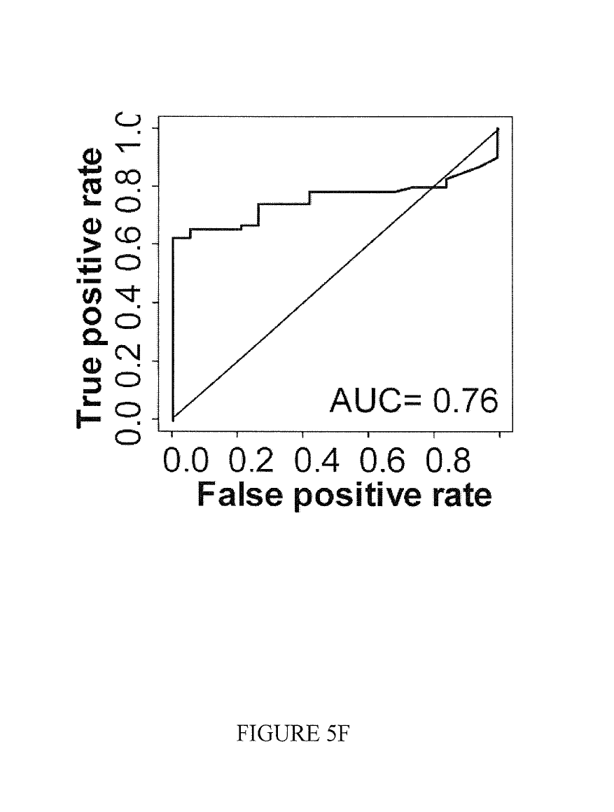

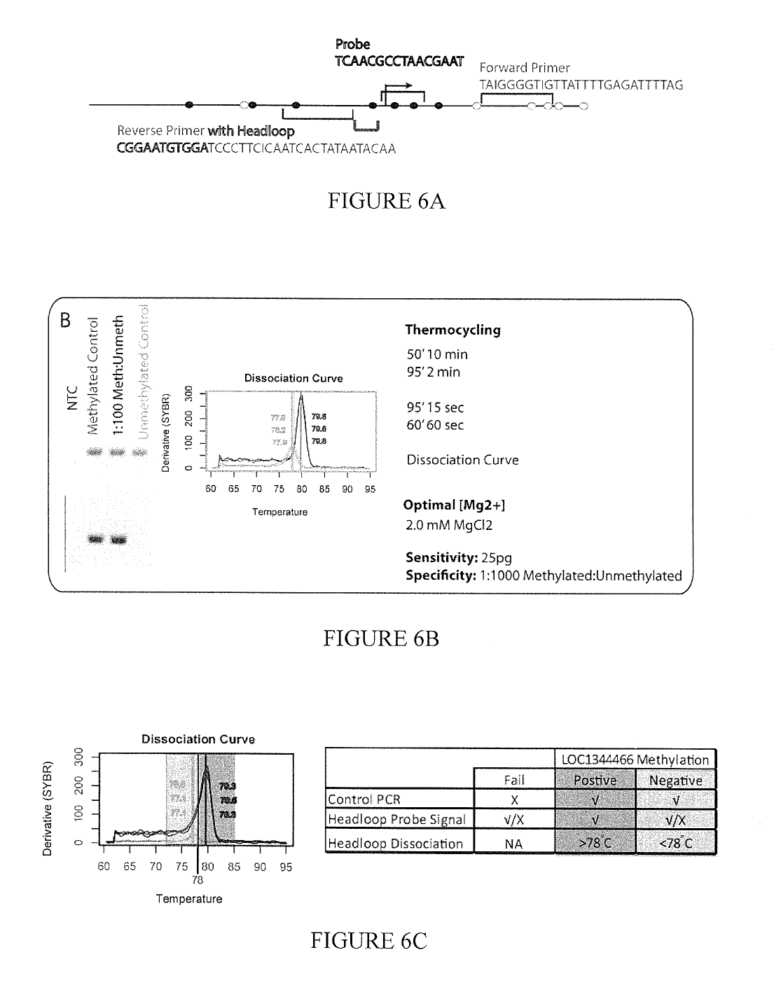

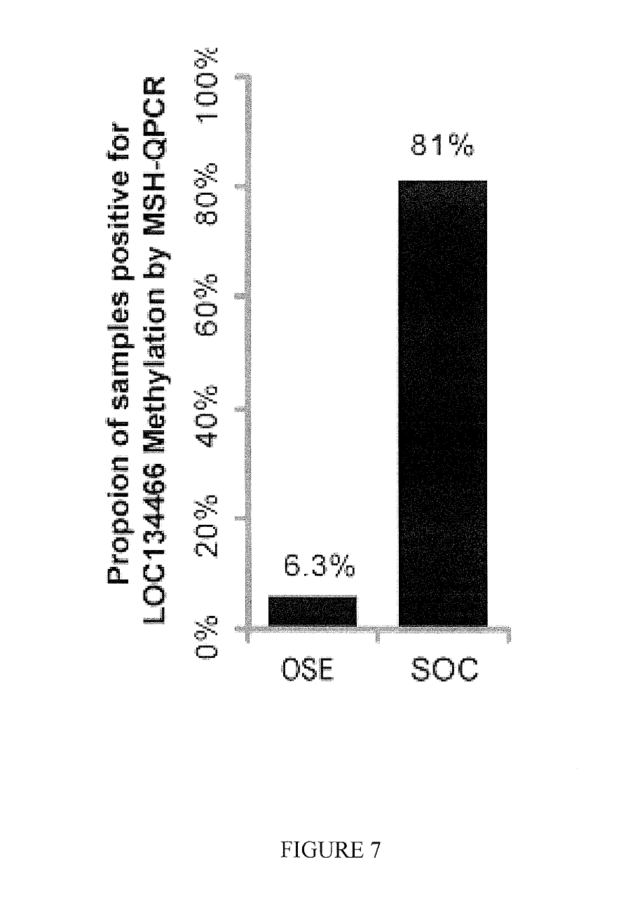

A more detailed analysis of LOC134466 methylation was performed using bisulphite clonal sequencing in five EOC and four OSE patient samples. An enrichment of methylation at 4 CpG dinucleotides at the TSS in tumors, relative to OSE DNA (73.6% & 10.3% respectively chi squared p>0.0001) was identified (FIGS. 5A AND 5b). In addition, methylation of the four CpG dinucleotides was commonly associated with gene repression in immortalized OSE, and EOC cell lines (FIG. 5C). LOC134466 methylation was quantified by Sequenom MassARRAY for 69 cancers and 19 OSE. Unsupervised hierarchical clustering (FIG. 5D) revealed that a high proportion (34/69, 49%) of cancers were hypermethylated at these 4 CpG sites relative to OSE (0/19; chi squared 13.25, p<0.0005). Averaged CpG methylation at these sites was significantly higher in cancers than OSE (p<0.005, FIG. 5E) and enabled discrimination of cancers versus OSE by ROC curve (AUC=0.76, FIG. 5F). To examine the potential of hypermethylation of the LOC134466 locus for detection of ovarian cancer, a methylation specific headloop suppression assay (MSH-PCR) was developed, to specifically interrogate the methylation status of the 4 TSS-associated CpG sites in archival formalin-fixed paraffin-embedded EOC relative to OSE. A diagrammatic representation of the MSH-PCR assay is depicted in FIG. 6A. MSH-PCR reaction conditions were optimized to distinguish methylated from unmethylated DNA (FIG. 6B) using fully methylated (Chemicon International, Temecula Calif., USA) and unmethylated control (OV90) DNA. Triplicate MSH-PCRs were performed on bisulphite converted DNA from 100 FFPE EOC and 15 EOC. The melting temperature (Tm) of the amplicon was calculated from the derivative SYBR signal during a heat dissociation cycle. Samples were considered methylated with a Tm of >78° C. as compared to the fully methylated control DNA (FIG. 6C). The results of this assay performed using archival formalin-fixed paraffin-embedded EOC relative to OSE are shown in FIG. 7. Hypermethylation was observed in 81% (81/100) of cancers relative to 6.3% of OSE (1/15). These results provide evidence of deregulation of LOC134466 in cancer and shows that DNA methylation is a potential mechanism associated with RNA suppression in a high proportion of type II EOC tumors.

TABLE 3
Candidate genes chosen for validation as methylated biomarkers of ovarian cancer

| | HGU133 Plus 2 | Re-expressed by | Fold Change | CpG | Fold Change | Reason for |
|---|---|---|---|---|---|---|
| Symbol | Probeset | 5aza in cell line | 5-aza-dC vs | Island | EOC vs OSE | inclusion |
| HSPA2 | 211538_s_at | A2780 | 33.78 | Yes | −3.29 | |
| KLF4 | 220266_s_at | CaOV3 | 3.78 | Yes | −8.85 | |
| LOC134466 | 244289_at | CaOV3 | 1.18 | Yes | −2.92 | |
| LTBP2 | 223690_at | A2780 | 1.57 | Yes | −3.02 | |
| PAPLN | 226435_at | A2780 | 2.78 | Yes | −4.2 | |
| PARVA | 222454_s_at | A2780 | 1.78 | Yes | −2.51 | |
| PEG3 | 209242_at | A2780 | 3.87 | Yes | −16.13 | |
| PTGER | 213933_at | CaOV3 | 1.32 | Yes | −4.69 | |
| SCIN1 | 1552365_at | A2780 | 1.28 | Yes | −2.26 | |
| SPOCK2 | 202524_s_at | A2780 | 1.41 | Yes | −2.99 | |
| TLE4 | 204872_at | CaOV3 | 1.20 | Yes | −6.49 | |
| ZNF177 | 207417_s_at | A2780 | 1.61 | Yes | −1.75 | |
| ZNF542 | 239250_at | A2780 | 2.35 | Yes | −2.11 | |
| ARMXC1 | 218694_at | A2780 | 1.19 | No | −4.44 | Lost In Ovarian cancers (a) |
| SGNE1 | 203889_at | A2780 | 4.15 | No | −4.55 | Epigenetically Silenced In Medulloblastoma (b) |
| IL18 | 206295_at | A2780 | 1.20 | No | −4.72 | Anti-tumour activity in OvCa (c) |
| BMP6 | 206176_at | CaOV3 | 1.25 | Yes | NA | Methylated in Breast Cancer (d) |
| CST6 | 231248_at | CaOV3 | 1.96 | Yes | NA | Methylated in Breast Cancer (d) |
| ICAM4 | 207194_s_at | CaOV3 | 1.89 | Yes | NA | Located in a genomic region of interest |
| PYCARD | 221666_s_at | A2780 | 3.72 | Yes | NA | Methylated biomarker of Ovarian Cancer (e) |
| SOCS1 | 210001_s_at | A2780 | 4.80 | Yes | NA | Methylated In Myeloma/Methylated in Ovarian Cancer (f) |

### Example 2

### LOC134466 is a Marker of Breast Cancer and Prostate Cancer

### 2.1 Methods

A headloop suppression PCR assay targeting the TSS of LOC134466 was performed substantially as described in Example 1. Assays were performed using bisulphite converted DNA from FFPE samples of 34 matched breast tumor and lymph-node metastases and samples from another cohort of nine matched normal, ductal carcinoma in situ (DCIS) and breast tumor samples. In addition, bisulfite converted DNA from normal and cancer cell line DNA were tested for LOC134466 methylation by headloop suppression PCR and by Sequenom MassARRAY.

### 2.2 Results

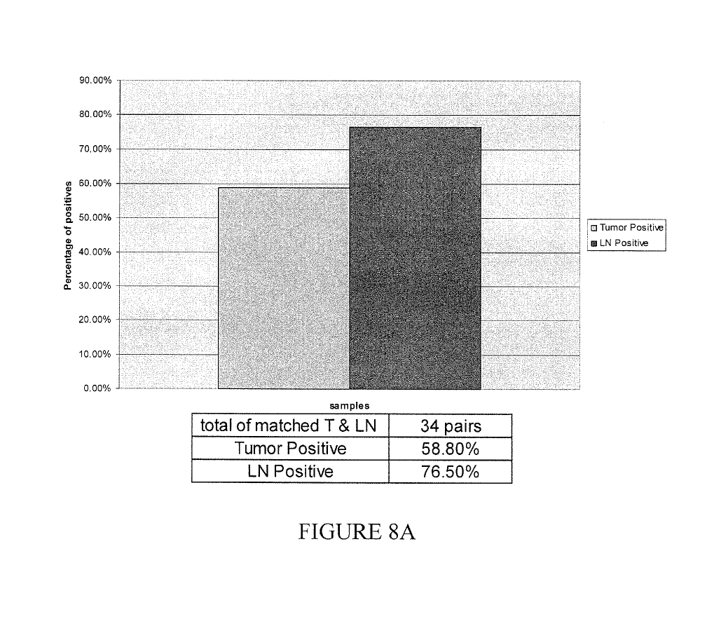

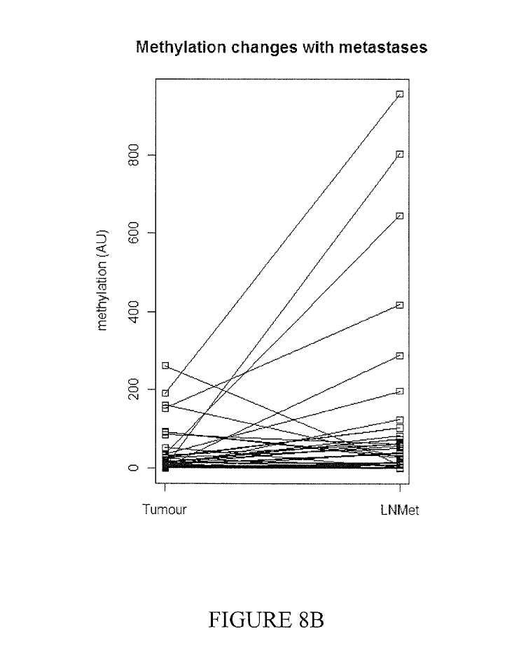

Results of a headloop suppression assay detecting methylation at the TSS of LOC134466 were performed on bisulfite converted DNA from FFPE samples of breast tumours and lymph-node metastases are shown in FIG. 8. The proportion of samples scoring positive for methylation by headloop assay was calculated. LOC134466 was shown to be methylated in almost 60% of breast tumours. Interestingly, LOC134466 methylation was shown to occur in a higher proportion of matched lymph node metastases (76%). Without being bound by any theory or mode of action, this pattern of methylation may indicate an involvement in dissemination of the tumor.

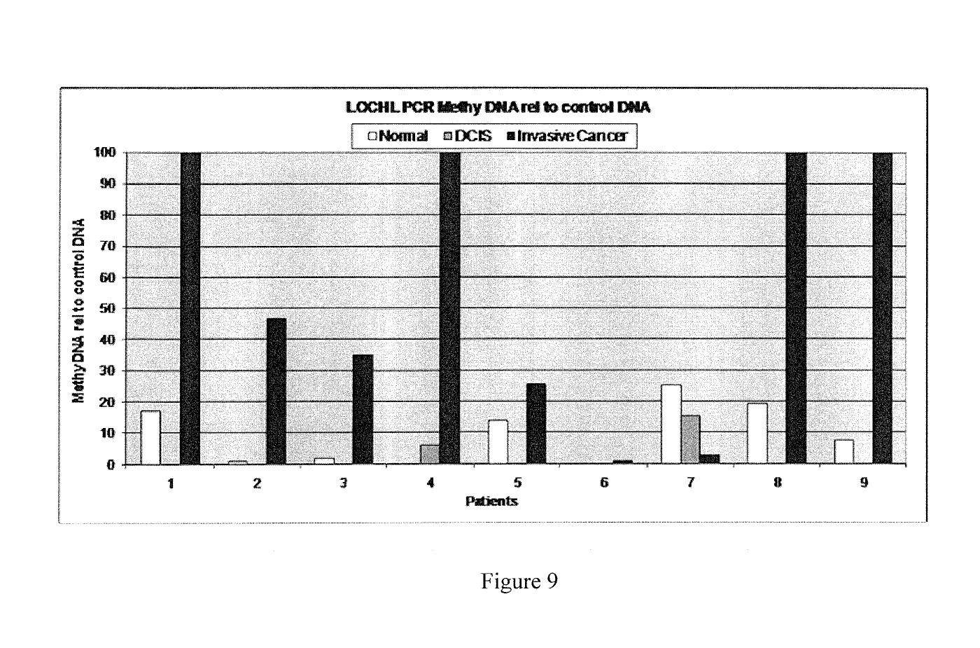

The headloop suppression assay was also used to detect methylation of LOC134466 in matched FFPE samples of normal epithelium, DCIS and breast tumours. Results are shown in FIG. 9. The relative methylation of each sample was calculated by comparing methylation by headloop assay to the amount of input bisulphite converted DNA (as measured by control PCR). In general, relative methylation increased with oncogenic progression indicating that LOC134466 methylation occurs early in cancer development as well as increasing during progression.

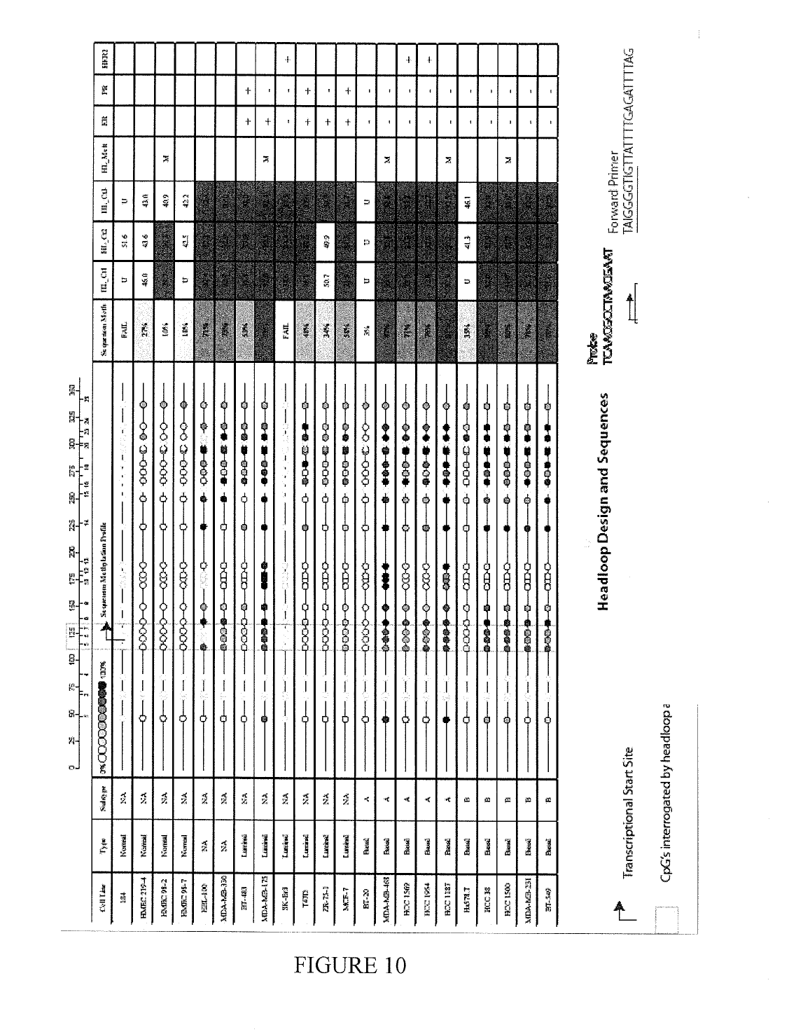

Bisulphite converted DNA from cell lines derived from normal breast (n=4), luminal invasive carcinoma (n−6) and basal carcinoma (n=10) were also tested for methylation levels by both sequenom massARRAY and headloop suppression PCR described above. Results are shown in FIG. 10. There was a strong correlation between both methods and breast cancer cell lines were observed to be more frequently hypermethylated than normal cell lines.

The increased methylation of CpG islands in LOC134466 is associated with reduced expression of this gene in breast cancers. Similar reduced expression is also observed in prostate cancer.

### Example 3

### Additional Analysis of LOC134466 Methylation in Ovarian Cancer

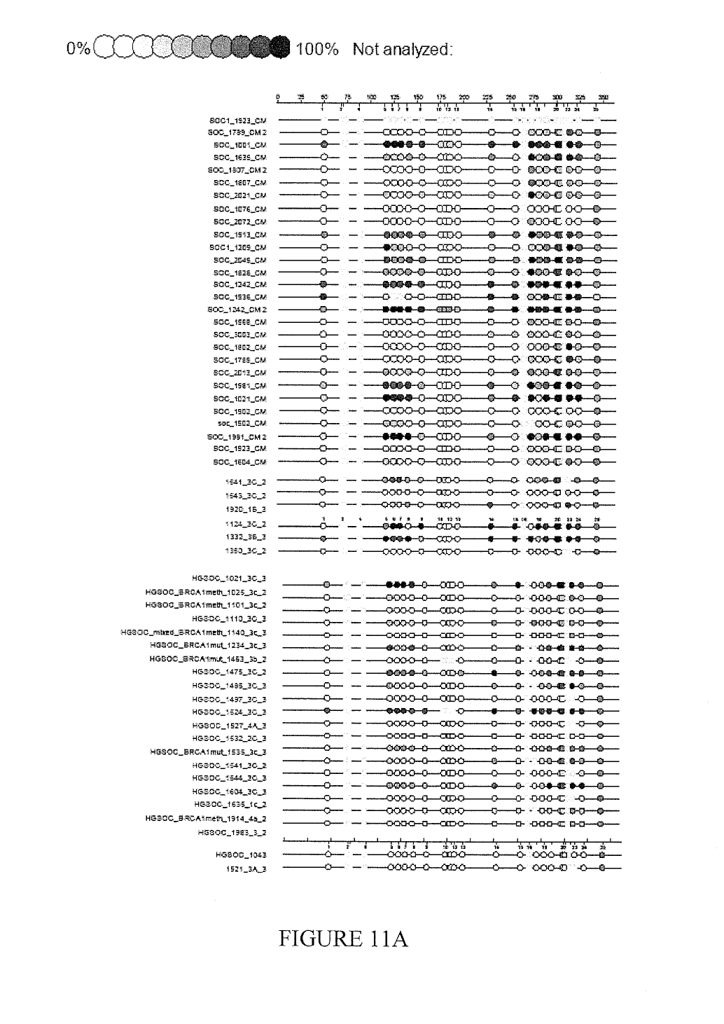

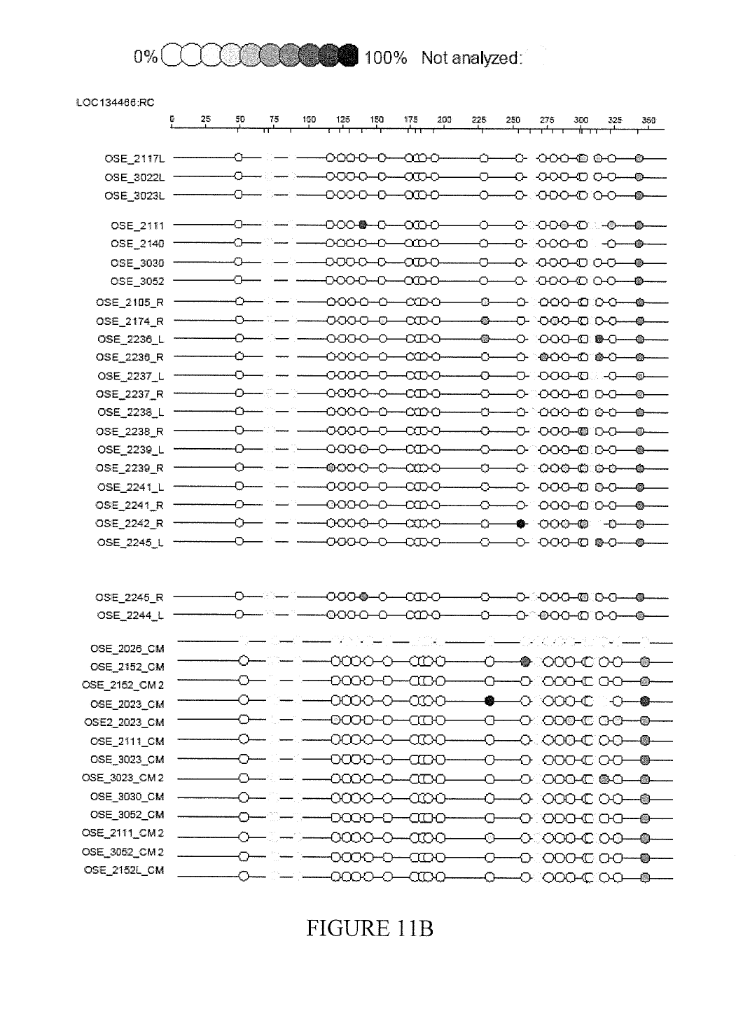

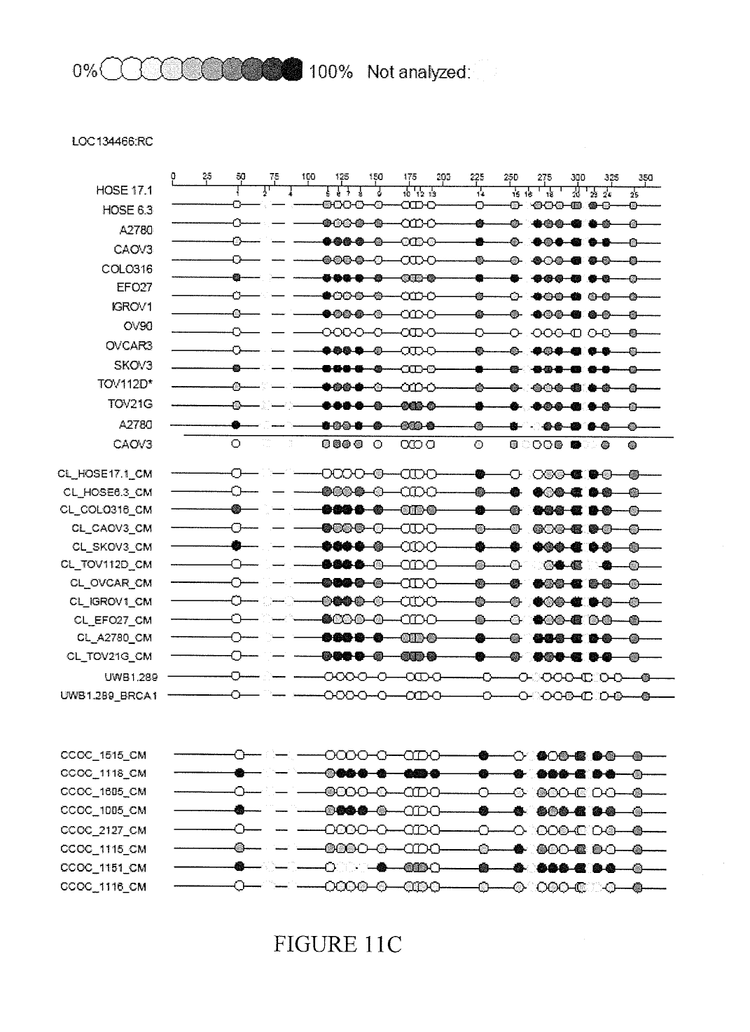

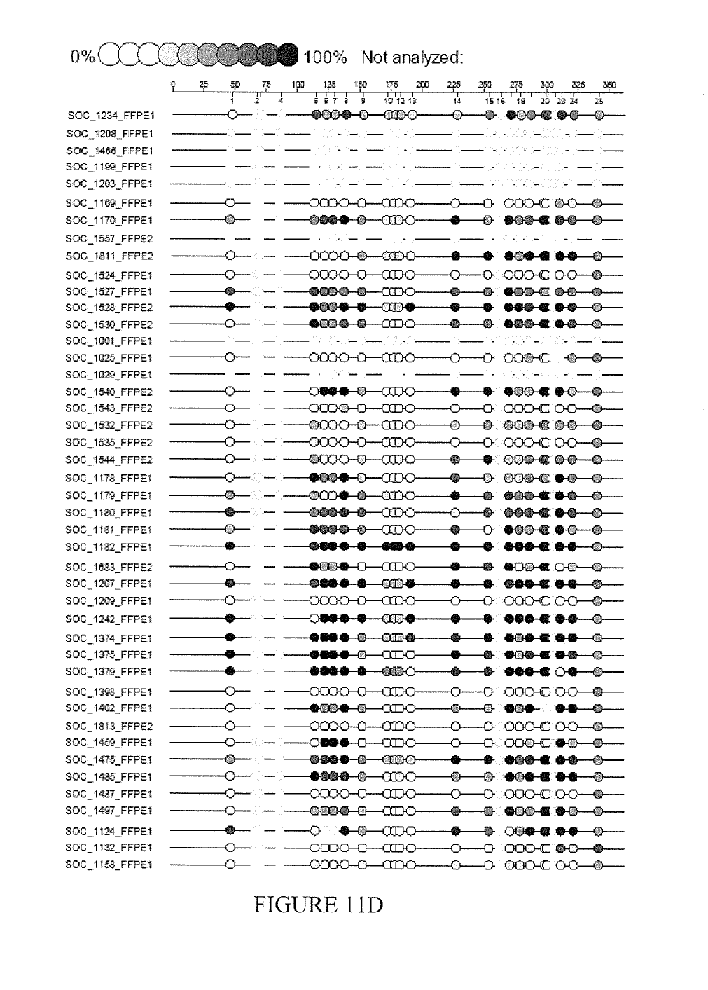

Sequenom massARRAY quantitative methylation analysis was performed substantially as described in Example 1 to determine the degree of methylation of CpG islands in LOC134466 in fresh frozen ovarian cancer samples, normal OSE, immortalized ovarian surface epithelium cancers and archival formalin fixed paraffin embedded ovarian cancer samples. Results are shown in FIGS. 11A-11D. These results show that methylation is increased in cancer samples and that the level of methylation can be determined in samples processed in various manners.

### Example 4

### Genomic Regions Surrounding LOC134466 are Methylated in Ovarian Cancer

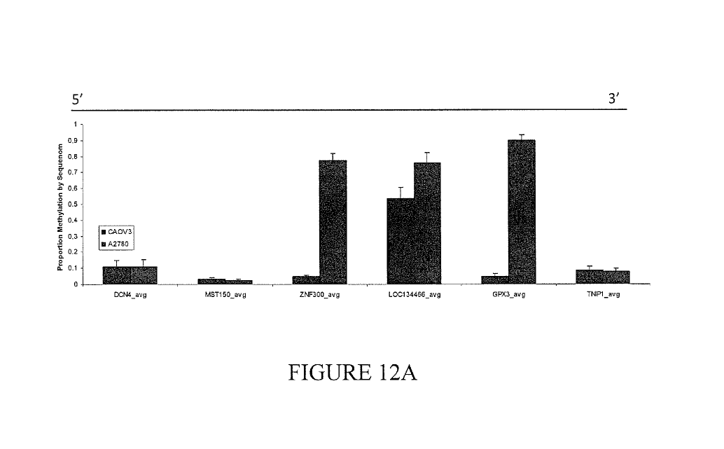

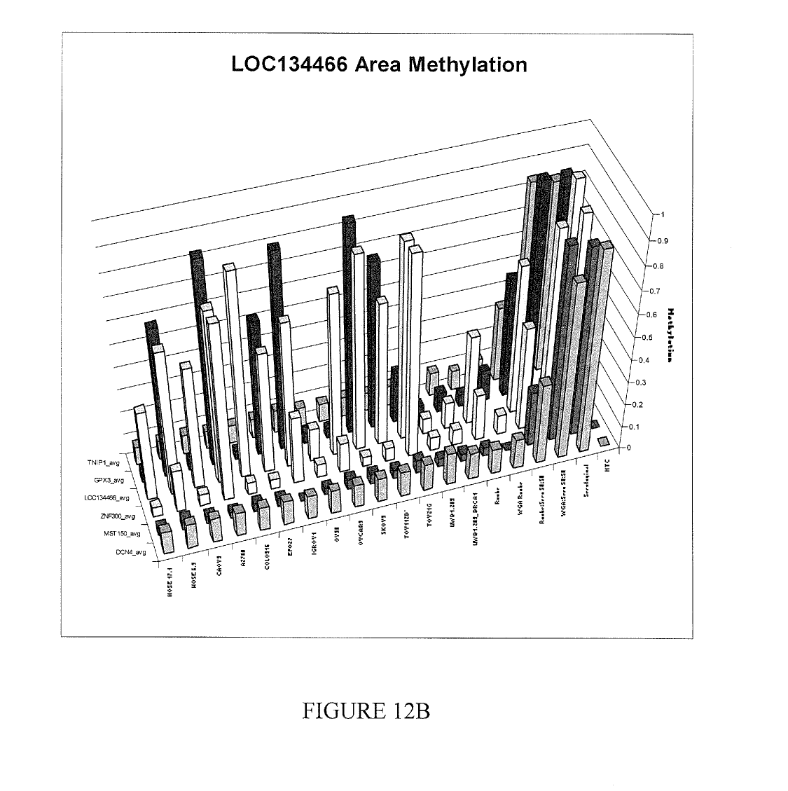

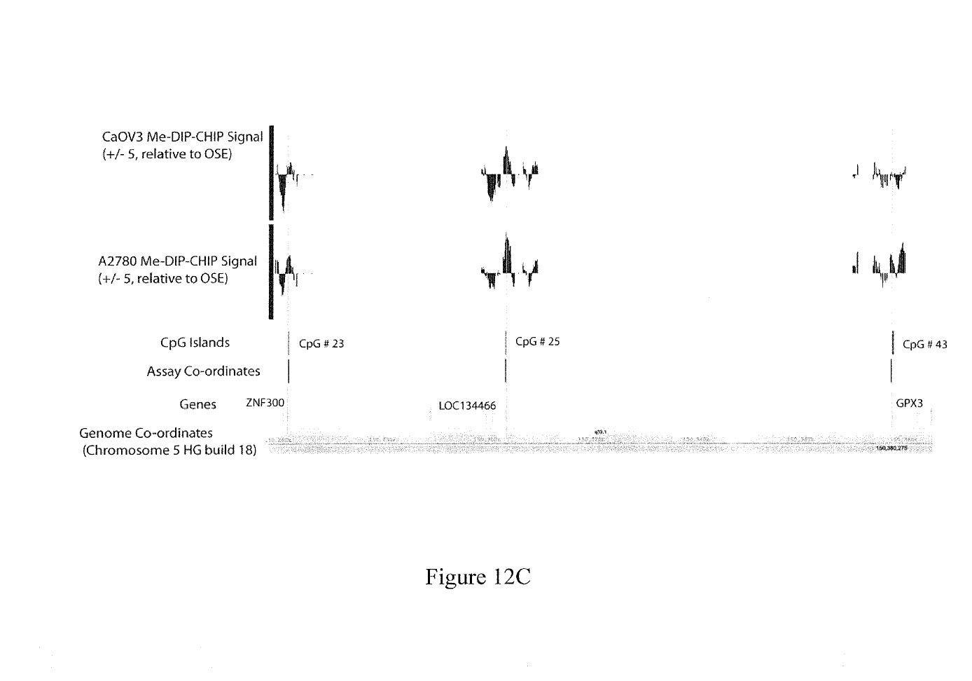

Sequenom massARRAY quantitative methylation analysis was performed substantially as described in Example 1 to determine the degree of methylation of CpG islands surrounding LOC134466 in cell lines. MeDIP-Chip analysis was also performed. As shown in FIGS. 12A and 12B, CpG islands in genes surrounding LOC134466 was increased in cancer samples. FIG. 12C indicated hypermethylation of CpG#23, CpG#25 and CpG#43 in the cell lines A2780 and CaOV3 relative to ovarian surface epithelium (OSE) cells as measured by MeDIP-chip. Sequenom massARRAY quantitative methylation analysis was performed substantially as described in Example 1 to determine the degree of methylation of CpG islands surrounding LOC134466 in other cell lines. As shown in FIGS. 12A and 12B, methylation of CpG islands in genes surrounding LOC134466 was increased in multiple cell lines.

### Example 5

### Additional Analysis by Headloop PCR of Gene Methylation in Ovarian Cancer

### 5.1 Methods

159 archival formalin-fixed paraffin embedded (FFPE) ovarian tumour samples were processed at the Royal Hospital for Women (Randwick, Australia) and blocks were acquired for DNA methylation analysis. A section from each tumour sample was stained with Haematoxylin and Eosin and regions containing >80% tumour were marked and three 1 mm diameter by 2-3 mm length cores were removed for DNA extraction with Gentra Puregene DNA isolation kit (Qiagen) according to manufacturer's instructions.

Normal primary ovarian surface epithelium (OSE) samples were obtained with consent, by scraping the surface of normal ovaries that were removed during surgery for nonovarian gynaecological malignancies followed by establishment of epithelial cells in culture. Cultures were evaluated for purity by staining for high molecular weight cytokeratin to exclude stromal contamination and maintained in culture as previously described (Rosen et al., Methods Enzymol. 2006; 407:660-676, 2006). Cell pellets from passage three or earlier were processed for DNA (˜1E6 cells with QiaAMP mini kits (Qiagen, Almeda Calif., USA)).

DNA from 1-2 μg FFPE, OSE or control DNA was bisulfite converted using the EZ-96 Methylation Gold Kit (Zymo Research, Irvine Calif., USA).

Triplicate methylation-specific headloop PCR (MSH-PCR) assays were performed on bisulfite converted DNA from 159 FFPE EOC and 15 OSE (under conditions set out in Table 4 below). The primer sequences used are set out in Table 5 below. The melting temperature (Tm) of the amplicon was calculated from the derivative SYBR signal during a heat dissociation cycle. The control assay for presence of bisulfite converted DNA was performed using rDNA assay (under conditions as set out in Table 6 below).

Positive signal for methylation was determined if a sample both passed the control PCR assay and had two or more replicate reactions amplify in the PCR stage with a Ct of <45 and resultant PCR product had a peak melting temperature of more than the cutoffs set out in Table 7 below (Optimisation of MSH-PCR).

TABLE 4
PCR components and thermocycling for MSH-PCR assays.

| MSH-PCR | x1 |
|---|---|
| 10x Platinum Taq Buffer | 1 |
| SYBR (1:1000) | 0.08 |
| Platinum Taq Polymerase | 0.06 |
| 2 mM dNTP | 1 |
| 50 mM MgCL2 | To desired |
| 5 uM F | 0.6 |
| 5 uM R | 0.6 |
| 5 uM probe | 0.3 |
| Total Volume | 15 |

Thermocycling

| Temperature | Time | | Cycles |
|---|---|---|---|
| 95 | 2 | m | 1 |
| 95 | 15 | s | 50 |
| Annealing Temp | 60 | s | |
| 95 | 15 | s | Heat |
| 60 | 15 | s | Dissoci. |

| | |
|---|---|
| 95 | Slow Ramp |

TABLE 5
Primer sequences used for MSH-PCR assays.

| | | | Assay | | Annealing |
|---|---|---|---|---|---|
| Name | Sequence | Gene | Type | Type | Temperature |
| ARMXC_HL_F1 | GATGGGAGTGGTAATTGGGGTTT | ARMCX1 | MSH-PCR | F1 | 60 |
| ARMXC_HL_R1 | ATACAACCIAACCAAACIAAAACTAAA | ARMCX1 | | R1 | |
| ARMXC_HL_R1HL | TGTGTAGATGTTTTTTTAATATACAACCIAACCAAACIAAAACTAAA | ARMCX1 | | R1HL | |
| ARMXC_HLcomPrb | AACGTCTACACGCGTCGA | ARMCX1 | | Probe | |
| ICAM4_HL_F1 | GGGTTGIGGGTTTTTTTATTTTTAGAGT | ICAM4 | MSH-PCR | F1 | 62 |
| ICAM4_HL_F1HL | ACACCAAAAAACCAAAACCAAGGGTTGIGGGTTTTTTTATTTTTAGAGT | ICAM4 | | F1HL | |
| ICAM4_HL_R1 | AACACAAAAAAAACAAAAACCCCATAA | ICAM4 | | R1 | |
| ICAM4_HL_R1HL | TGGGTTTTTTGAACACAAAAAAAACAAAAACCCCATAA | ICAM4 | | R1HL | |
| ICAM4_HLcomPrb | AACCCCGCGCCAAA | ICAM4 | | Probe | |
| LOC_ts_R4HL | TGGAATGTGGATCCCTTCICAATCACTATAATACAA | LOC134466\ | MSH-PCR | R4HL | 60 |
| LOC_ts_F4 | TAIGGGGTIGTTATTTTGAGATTTTAG | LOC134466\ | | F4 | |
| LOC_ts_R4 | TCCCTTCICAATCACTATAATACAA | LOC134466\ | | R4 | |
| LOC_HLcomPrb | TCAACGCCTAACGAAT | LOC134466\ | | Probe | |
| PEG3_HL_F1 | TTGTATTTIGGTGTAGAAGTTTGGGTAGT | PEG3 | MSH PCR | F1 | 58 |
| PEG3_HL_R1 | CICCTAACICACCCTCATAA | PEG3 | | R1 | |
| PEG3_HL_R1HL | TGTTGTTGGGTGTTGGGTGCICCTAACICACCCTCATAA | PEG3 | | R1HL | |
| PEG3_HLcomPrb | AATAAACATCTCCCGCGCC | PEG3 | | Probe | |
| PYCARD_HL_F1 | TTTCGGGTTTTATTTCGTAGGITAGT | PYCARD | MSH PCR | F1 | 58 |
| PYCARD_HL_R1 | CTCCCACCCAAACCTCTAAATTAA | PYCARD | | R1 | |
| PYCARD_HL_R1HL | TGATGGTTTGGGGTTCTCCCACCCAAACCTCTAAATTAA | PYCARD | | R1HL | |
| PYCARD_HLcomPrb | CGTCGAAAAACGCCA | PYCARD | | Probe | |
| SGNE1_HL_F1 | GAGTTTTGGIGAGGTAGTTTTTATTTGT | SGNE1 | MSH-PCR | F1 | 60 |
| SGNE1_HL_R1 | CCAAAAACIAACIAACTAAAAAACTAATA | SGNE1 | | R1 | |
| SGNE1_HL_R1HL | TGGAGGAGGGTGATGCCAAAAACIAACIAACTAAAAAACTAATA | SGNE1 | | R1HL | |
| SGNE1_HLcomPrb | CATCTTAACTCCGCCCCGA | SGNE1 | | Probe | |

TABLE 6
PCR components and thermocycling for rDNA control PCR assay.

| rDNA control PCR | x1 |
|---|---|
| 10x Platinum Taq Buffer | 1.5 |
| 2 mM dNTP | 1.5 |
| 50 mM MgCL2 | 0.45 |
| 5 uM MSECF1FL For | 0.6 |
| 5 uM MSECR1 Rev | 0.6 |
| SYBR (1:1000) | 0.1 |
| Platinum Taq Polymerase | 0.1 |
| Total Volume | 15 |

Thermocycling

| Temperature | Time | | Cycles |
|---|---|---|---|
| 95 | 2 | m | 1 |
| 95 | 15 | s | 5 |
| 50 | 40 | s | |
| 72 | 20 | s | |
| 95 | 15 | s | 45 |
| 60 | 40 | s | |
| 72 | 20 | s | |

TABLE 7
Optimisation results for rDNA control PCR and MSH-PCR assays.

| Gene | Negative | [Mg] | Ta | Sensitivity | Efficiency | Specificity | Temp. Cutoff |
|---|---|---|---|---|---|---|---|
| ICAM | ROCHE | 2.0 mM | 62 | 50 pg | 62% | 1:500 | 79.5° |
| SGNE1 | CaOV3 | 2.25 mM | 62 | 50 pg | 58% | 1:2000 | 81° |
| ARMXC1 | CaOV3 | 2.25 mM | 60 | 75 pg | 78% | 1:2000 | 79.5° |
| PEG3 | TOV112D | 2.0 mM | 58.5 | 25 pg | 100% | 1:1000 | 86.5° |
| PYCARD | ROCHE | 2.25 mM | 58.5 | 25 pg | 86% | 1:1000 | 80.25° |
| LOC134466 | OV90 | 2.0 mM | 60 | 25 pg | 92% | 1:1000 | 78° |

### 5.2 Results

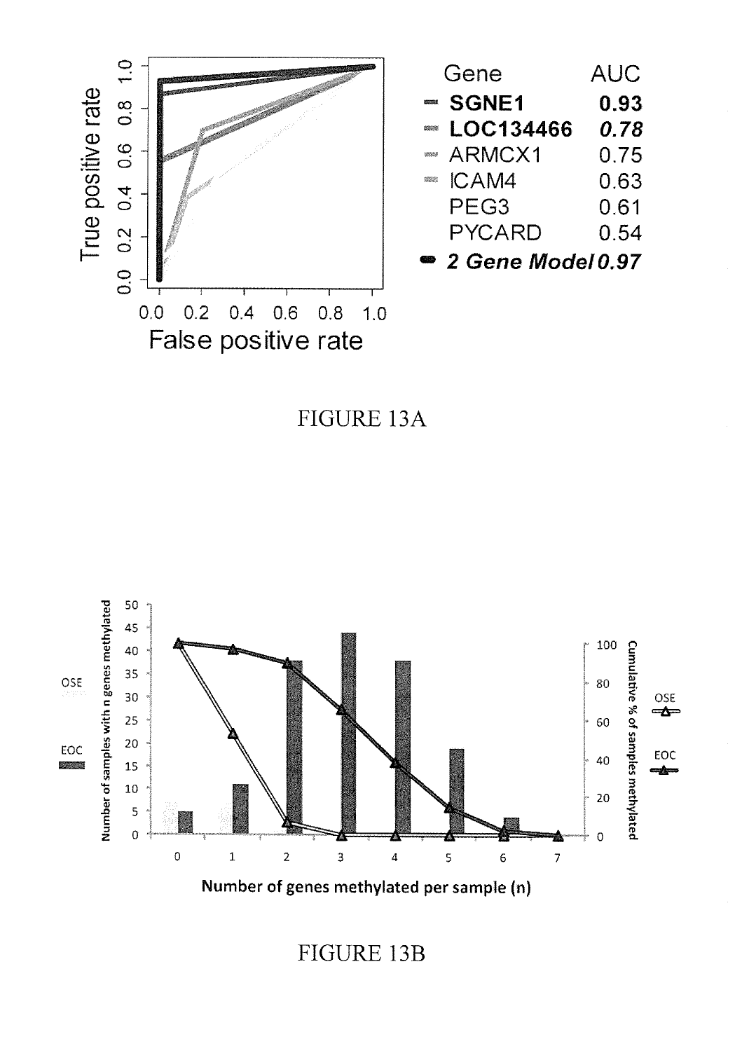

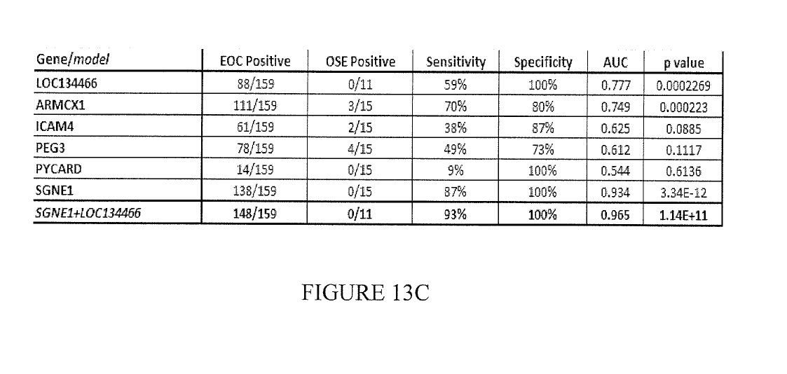

Results of the MSH-PCR assay detecting methylation in several genes on bisulfite converted DNA from FFPE samples are shown in FIG. 13. As shown in FIG. 13A, the methylation status of each of the genes LOC134466, SGNE1, ARMCX1, ICAM4, PEG3 and PYCARD proved effective in detecting ovarian cancer, with LOC134466 and SGNE1 each being particularly effective when considered alone. In addition, combined analysis of the methylation status of LOC134466 and SGNE1 together resulted in an AUC of 0.97, demonstrating that the combination of these two markers is particularly effective in detecting ovarian cancer.

FIG. 13B illustrates the distribution of methylation status of the six gene panel in EOC vs OEC. The cumulative percentage of samples containing at least n numbers of methylated genes within the six gene panel indicates that the analysis of n=two, three, four, five or six of the six gene panel reliably distinguishes ovarian cancer from healthy tissue.

Raw data and calculated sensitivity and specificity for each of the genes, and for the combined analysis of LOC134466 and SGNE1 together is illustrated in FIG. 13C.

### Example 6

### LOC134466 Methylation Status in Plasma Samples

### 6.1 Methods

Blood samples from healthy subjects were obtained with consent at the Garvan Institute (Ethics Approval 09/100). Other blood samples were obtained in EDTA from patients with either EOC or non-gynaecological malignancies (normal OSE) preoperatively by anaesthetists at the Royal Hospital for Women (Randwick, Australia). Cell free plasma was separated from whole blood by centrifugation (3000 rpm, 10 min) and stored at −80° C. for processing. DNA was purified from 1 ml of plasma using QiaAMP DNA mini kits (Qiagen) and bisulfite converted using the Epitect conversion kit (Qiagen). Presence of bisulfite converted DNA was performed by the SFN MSH-PCR as described previously (Montavon et al. Gynecol Oncol. 2012 March; 124(3):582-8.) The LOC13446 or SGNE1 headloop (as previously described for FFPE) was applied to converted DNA and average probe Ct used to assess methylation status. A positive result was defined as positive for control headloop (SFN) and 2 or more replicate PCR positive results with Ct<45.

### 6.2 Results

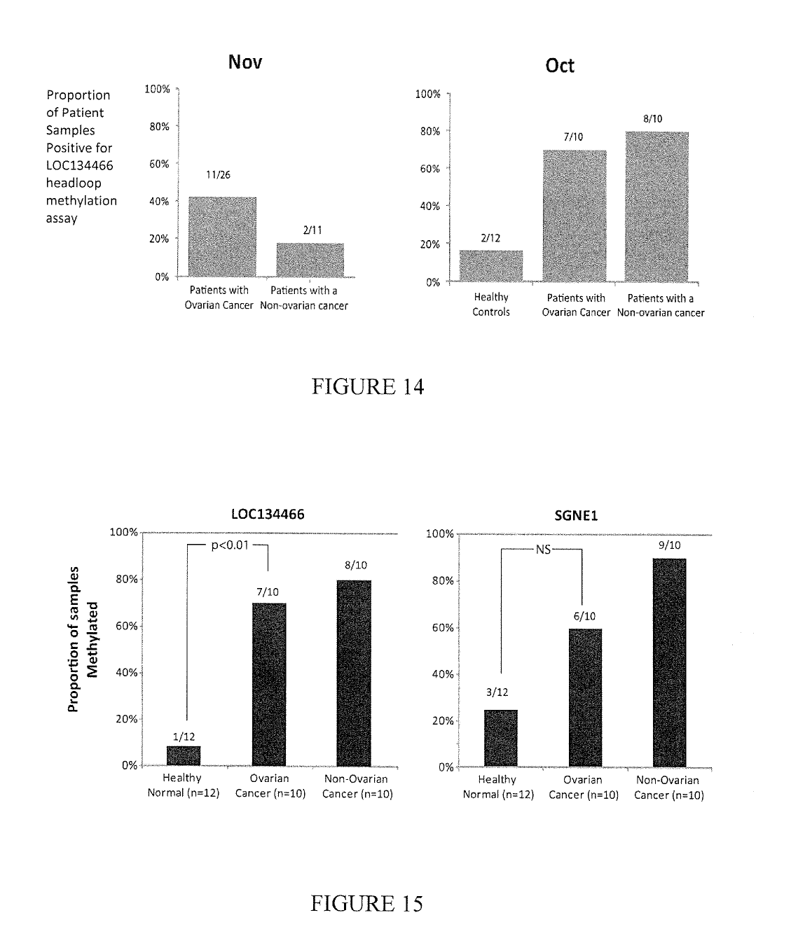

The results of two separate experiments determining LOC134466 methylation status (one comparing plasma from 26 patients with ovarian cancer to plasma from 11 patients with non-ovarian cancers (Nov); the other comparing plasma from 12 healthy control subjects, 10 patients with ovarian cancer and 10 patients with non-ovarian cancers (Oct)) are shown in FIG. 14. These results demonstrate that LOC134466 methylation status can be detected in plasma samples, and that LOC134466 methylation status predicts cancer with a sensitivity of approximately 50% in the samples tested and when applying the cut-offs designed in these experiments. The sensitivity may have reflected the experimental conditions under which the assays were performed, for example, the low levels of material input. The use of greater quantities of genetic material to perform the assay and/or the performance of the assay on greater numbers of samples may demonstrate an increased sensitivity.

FIG. 15 illustrates the proportion of plasma samples from OSE and EOC patients positive for LOC134466 or SGNE1 methylation, comparing plasma from 12 healthy control subjects, 10 patients with ovarian cancer and 10 patients with non-ovarian cancers. Although SGNE1 proved less sensitive in these samples and using the cut-offs designed in these experiments, these results indicate that a combined analysis of LOC134466 and SGNE1 methylation status in plasma samples is likely to be particularly effective in reliably distinguishing subjects suffering from cancer from normal, healthy subjects. Again, the sensitivity may have reflected the experimental conditions under which the assays were performed, for example, the low levels of material input. The use of greater quantities of genetic material to perform the assay and/or the performance of the assay on greater numbers of samples may demonstrate an increased sensitivity.

### Example 7

### LOC134466 and SGNE1 Methylation Status in Cancer Cell Lines

### 7.1 Methods

Cell line DNA was extracted using the Stratagene DNA extraction Kit (Agilent, Santa Clara Calif., USA) according to manufacturer's instructions. 1 microgram DNA was bisulfite converted using the EZ-96 Methylation Gold Kit (Zymo Research, Irvine Calif., USA).

Triplicate MSH-PCRs were performed on bisulfite converted DNA. The melting temperature (Tm) of the amplicon was calculated from the derivative SYBR signal during a heat dissociation cycle. The control assay for presence of bisulfite converted DNA was performed using rDNA assay.

A positive result was defined by 2 or more replicate PCR positive results with Ct<45 and a peak amplicon melting temperature of >78° C. for LOC134466 and >81° C. for SGNE1.

### 7.2 Results

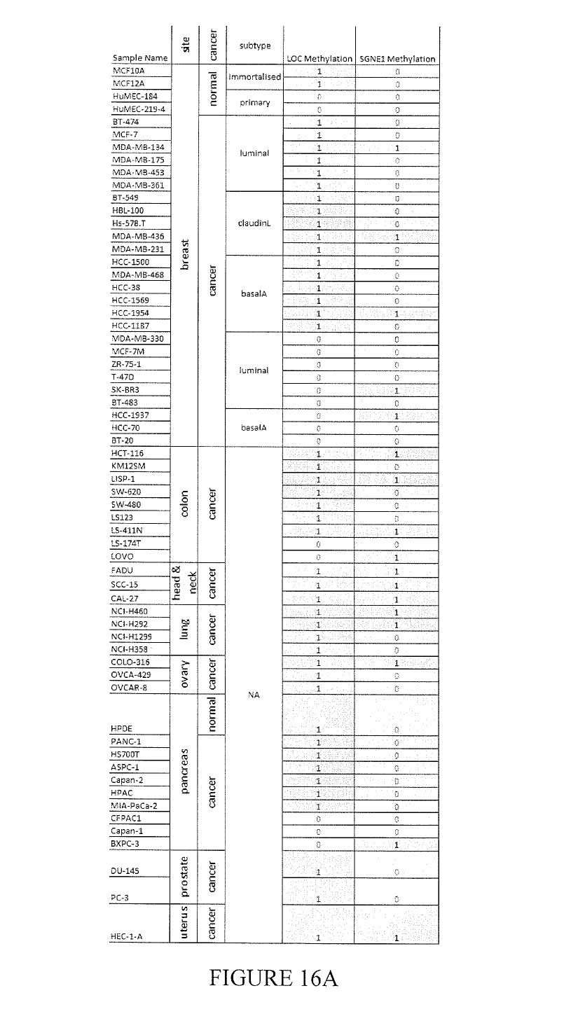

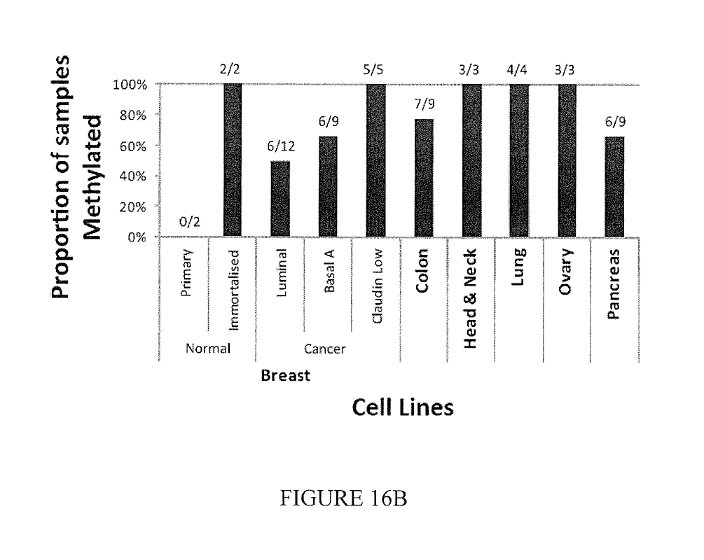

The methylation status of both LOC134466 and SGNE1 in specific cell lines is shown in FIG. 16A (1, dark shading=methylated; 0, light shading=unmethylated). The results for LOC134466 alone are illustrated in FIG. 16B. These results indicate that LOC134466 is often methylated in a number of alternative cancer cell lines.

### Example 8

### LOC134466 Expression and Methylation Analysis in Cancer Cell Lines

### 8.1 Methods

Total RNA was extracted with Qiagen RNeasy mini kit (Qiagen, Almeda Calif., USA) and DNase treated using DNase (Ambion or Qiagen). Quantification of RNA was performed on a NanoDrop spectrophotometer (Thermo Scientific, Wilmington Del., USA).

500 ng RNA was then reverse transcribed into cDNA of mRNA (Oligo dT primers, Promega, Madison Wis., USA) for qPCR of LOC13446 expression (Taqman AssayID Hs00859547_m1) relative to GAPDH using the 2^{-ΔCt} method under the conditions shown in Table 8 below.

TABLE 8
TaqMan ® qPCR and thermocycling conditions
for LOC134466 expression analysis.

| A) TaqMan qPCR | x1 |
|---|---|
| 2x Universal Gene Expression Mix | 5 |
| 20x Primer Probe | 0.5 |
| Water | 0.5 |
| Total Volume | 10 |

C) Thermocycling

| Temperature | Time | | Cycles |
|---|---|---|---|
| 50 | 2 | m | 1 |
| 95 | 10 | m | 1 |
| 95 | 15 | s | 45 |
| 60 | 60 | s | |
| 95 | 15 | s | Heat |
| 60 | 15 | s | Dissoci. |

| | |
|---|---|
| 95 | Slow Ramp |

### 8.2 Results

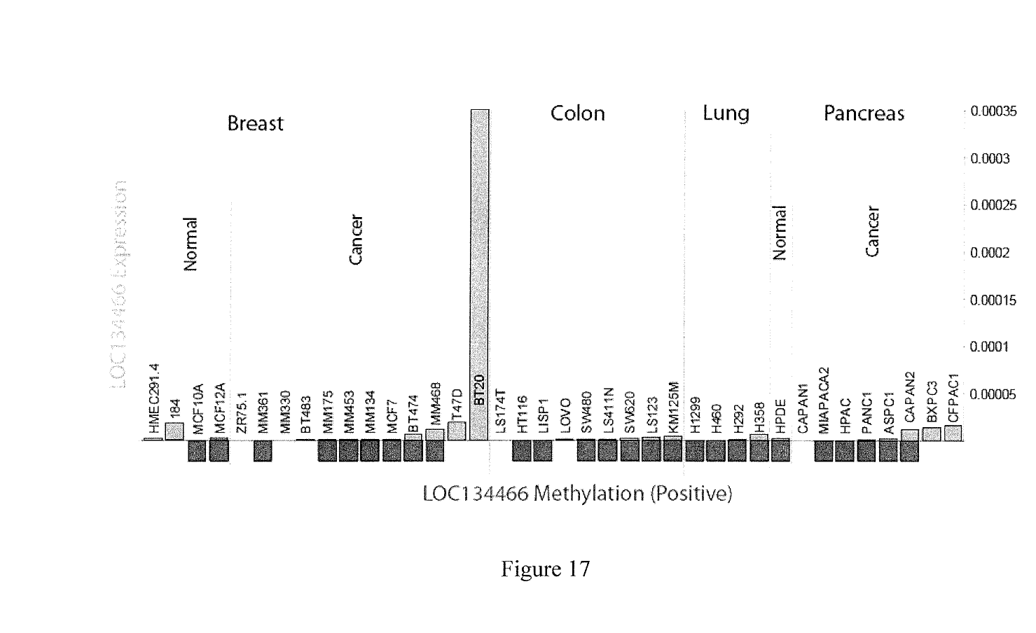

LOC134466 expression (as determined by TaqMan® qRTPCR) relative to DNA methylation (as determined by MSH-PCR) in the cell lines analysed is shown in FIG. 17. Notably, LOC134466 is expressed in primary breast normal cells (HMEC219 and 184) but not in immortalised breast cells (MCF10 and 12). In addition, most cancer cell lines contain methylated LOC134466 and do not express LOC134466. Conversely, most cell lines expressing LOC134466 are unmethylated.

### Example 9

### LOC134466 Methylation in Colon Cancer

### 9.1 Methods

Bisulfite treated DNA from colon cancers and adjacent normal epithelium from 42 patients were obtained from the Garvan Institute of Medical Research.

Methylation measured by LOC134466 MSH-PCR was calculated relative to a control (SFN; as in plasma experiments described in Example 6) with tumour methylation being normalised to 1 in order to estimate trends in relative methylation.

### 9.2 Results

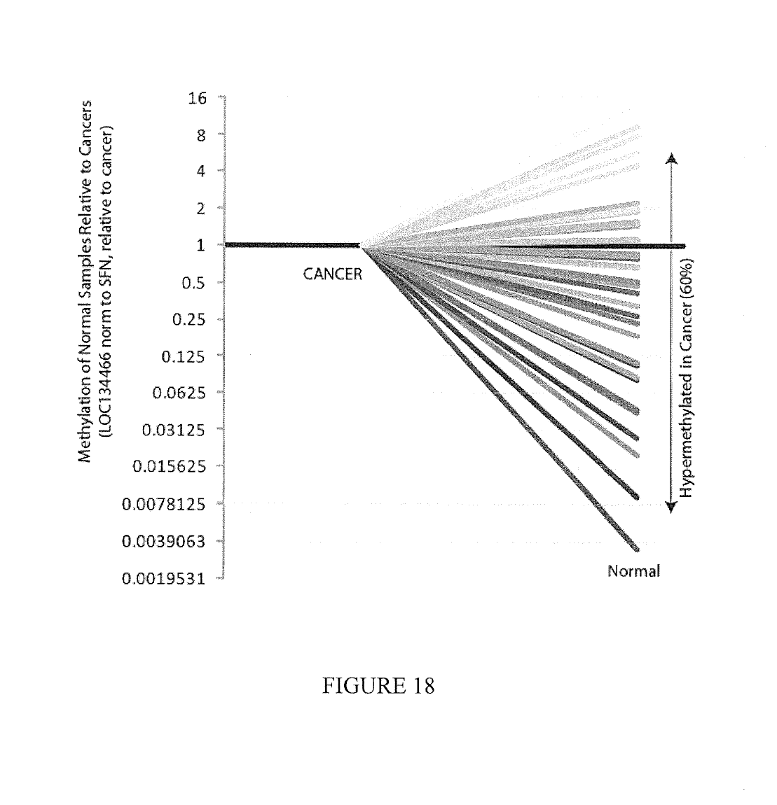

The results of the analysis of LOC134466 methylation status in colon cancer are shown in FIG. 18. Each diagonal line represents a matched normal tumour sample, and variation below 1 indicates hypermethylation in tumours relative to normals. LOC134466 appears to be hypermethylated in tumours relative to adjacent normals ˜60% of the time, indicating that LOC134466 is also reliably indicative of colon cancer.

1. A method for diagnosing a cancer or a predisposition thereto in a subject, the method comprising detecting in a sample from the subject:
(i) modified chromatin relative to a non-cancerous sample the modified chromatin being positioned within a locus containing a LOC134466 gene; and/or
(ii) modified expression relative to a non-cancerous sample of the LOC1334466 RNA,
wherein the modified chromatin and/or the modified expression is diagnostic of a cancer or a predisposition thereto in the subject.

2. (canceled)

3. A method for monitoring the efficacy of treatment of a subject receiving treatment for a cancer, the method comprising identifying and/or detecting in a sample from the subject:
(i) modified chromatin relative to a non-cancerous sample the modified chromatin being positioned within a locus containing a LOC134466 gene; and/or
(ii) modified expression relative to a non-cancerous sample of the LOC134466 RNA,
wherein the modified chromatin and/or the modified expression indicates that the treatment is not effective.

4. A method for determining the likelihood of survival of a subject suffering from a cancer, the method comprising identifying and/or detecting in a sample from the subject
(i) modified chromatin relative to a non-cancerous sample the modified chromatin being positioned within a locus containing a LOC134466 gene; and/or
(ii) modified expression relative to a non-cancerous sample of the LOC134466 RNA,
wherein the modified chromatin and/or the modified expression indicates that the subject is likely to survive.

5. The method of claim 1, additionally comprising detecting modified chromatin positioned within a gene or pseudogene and/or modified expression of the gene or pseudogene in the sample.

6. The method of claim 5, wherein the gene or pseudogene is selected from the group consisting of ARMCX1, ICAM4, IL18, PEG3, PYCARD, SGNE1, ZNF177, HSPA2, KLF4, LTBP2, PAPLN, PARVA, PTGER, SCIN1, SPOCK2, TLE4, ZNF542, BMP6, CST6, SOCS1 and combinations thereof.

7. The method of claim 5, wherein the gene is selected from the group consisting of ARMCX1, ICAM4, IL18, PEG3, PYCARD, SGNE1, ZNF177 and combinations thereof.

8. The method of claim 5, wherein the gene is selected from the group consisting of ARMCX1, ICAM4, PEG3, PYCARD, SGNE1 and combinations thereof.

9. The method of claim 5, wherein the gene is SGNE1 and/or ARMCX1 and/or PYCARD.

10. The method of claim 1, additionally comprising detecting modified chromatin positioned within ARMCX1, ICAM4, IL18, PEG3, PYCARD, SGNE1 and ZNF177 and/or modified expression of ARMCX1, ICAM4, IL18, PEG3, PYCARD, SGNE1 and ZNF177 in the sample.

11. The method of claim 1, comprising detecting modified chromatin positioned within ARMCX1, ICAM4, LOC134466, PEG3, PYCARD and SGNE1 and/or modified expression of ARMCX1, ICAM4, LOC134466, PEG3, PYCARD and SGNE1 in the sample.

12. The method of claim 1, wherein the modified expression is associated with the modified chromatin.

13. The method of claim 1, wherein the cancer is ovarian cancer, breast cancer, colon cancer or prostate cancer.

14. (canceled)

15. The method of claim 1, wherein modified chromatin is detected by performing a process comprising detecting the level of methylation of nucleic acid in the sample from the subject relative to a non-cancerous sample.

16. The method of claim 15, wherein the level of methylation is detected by performing a process comprising:
(i) detecting the level of methylation of a nucleic acid within the gene in the sample derived from the subject;
(ii) detecting the level of methylation of the nucleic acid within the gene in the control sample; and
(iii) comparing the level of methylation at (i) and (ii).

17. The method of claim 15, wherein the level of methylation is detected by one or more of the following:
(i) performing methylation-sensitive endonuclease digestion of DNA;
(ii) treating nucleic acid from the sample with an amount of a compound that selectively mutates non-methylated cytosine residues in nucleic acid under conditions sufficient to induce mutagenesis thereof and produce a mutant nucleic acid and amplifying the mutant nucleic acid using at least one primer that selectively hybridizes to the mutant nucleic acid;
(iii) treating nucleic acid from the sample with an amount of a compound that selectively mutates non-methylated cytosine residues in nucleic acid under conditions sufficient to induce mutagenesis thereof and produce a mutant nucleic acid, hybridizing a nucleic acid probe or primer capable of specifically hybridizing to the mutant nucleic acid and detecting the hybridized probe or primer; and
(iv) treating nucleic acid from the sample with an amount of a compound that selectively mutates non-methylated cytosine residues in nucleic acid under conditions sufficient to induce mutagenesis thereof and produce a mutant nucleic acid determining the nucleotide sequence of the mutant nucleic acid.

18. The method of claim 17, wherein compound that selectively mutates non-methylated cytosine residues is a salt of bisulphite.

19. The method of claim 15, wherein the level of methylation is detected in a nucleic acid comprising a sequence set forth in SEQ ID NO: 1 or 3.

20. The method of claim 1, wherein the level of expression of a nucleic acid is detected by performing a process comprising:
(i) detecting the level of expression of the gene in the sample from the subject; and
(ii) detecting the level of expression of the gene in the non-cancerous sample.

21. The method of claim 20, wherein the level of expression of the nucleic acid is detected by performing a process comprising hybridizing a probe or primer capable of specifically hybridizing to a transcript of the gene to the nucleic acid in a sample and detecting the level of hybridization by a detection means, wherein the level of hybridization of the probe or primer is indicative of the level of expression of the gene.

22. The method of claim 20, wherein the level of expression is detected by performing a process comprising contacting the sample with an antibody or antigen binding fragment thereof capable of specifically binding to a polypeptide encoded by the gene for a time and under conditions for a complex to form and detecting the level of the complex by a detection means, wherein the level of the complex is indicative of the level of expression of the gene.

23. The method of claim 1, wherein the sample comprises tissue and/or a body fluid suspected of comprising a cancer cell.

24. The method of claim 1, wherein the sample comprises tissue or cells from a breast, an ovary, a colon or a prostate.

25. The method of claim 23 wherein the body fluid is selected from the group consisting of whole blood, a fraction of blood such as blood serum or plasma, urine, saliva, breast milk, pleural fluid, sweat, tears and mixtures thereof.

26. The method of claim 1, wherein the non-cancerous sample is selected from the group consisting of:
(i) a sample comprising a non-cancerous cell;
(ii) a sample from a normal tissue;
(iii) a sample from a healthy tissue;
(iv) an extract of any one of (i) to (iii);
(v) a data set comprising measurements of modified chromatin and/or expression for a healthy individual or a population of healthy individuals;
(vi) a data set comprising measurements of modified chromatin and/or expression for a normal individual or a population of normal individuals; and
(vii) a data set comprising measurements of the modified chromatin and/or expression from the subject being tested wherein the measurements are determined in a matched sample having normal cells.

27. The method of claim 1, additionally comprising providing the result of the method.

28. (canceled)


SEQUENCE LISTING (132 sequences)

SEQ ID NO: 1 - DNA, 1174 bp, Homo sapiens
gcctgggatctcaaaatggcggccccgtgcggaaacagcgtctgggagcagtcatgttgcctcctgaacaaagccgctgaagatgaagaatgggcaaaatcgccccatacggaacagcgcagcctcgggagcccgttacctggctcgcgaacacgaagcgggagagttcgccaatatggatgtgacagcggttcccattaagcggtgataggatttttgggacctgataatcaagctagggacctgattcctcagacctctgccctatcggaagggaagcagaaaatgatcaggtctcagggtccagtgtcatttgaggatgtggctgtggatttcacccaggaggagtggcagcaactggactatgctcagaggaccctgtacagggatgtgatgctggagatctatagccacctggtctcaatgggatatccagtttccaaaccagatgtcatctccaagttggaacaaggagaagagccatggatcataaagagacacataccaaattggatctatccagacagagagagtagacttgacacccctcaactggatatatttagagatgttttcttccataaggagacactggaaagtattacaaggggtcattcattgtactccattttaaaagtctggcaaggtgatgaccagctggagagagatcaggaaaactgagacaaatttgtcaggcaagttgtagtcatcaacaacaaaagaatatctgaagagtcaggtcatccatataatatatttggaaaaatatttcatgactgcacagacctagatacttcaaaacaaagactgtgtaagtgtgattcatttgaaaagaccttgaaaccaaatattaacctagtgagttataataggaattttgcaagaaaaaacattgatgagaattttagatgtgggaaaacacctagctacagttcttgctattctaagcatgaaaaaaatcatagtggaatgatacactgtgaagctactcattgtggaaagattcttagcaataaacaatctcttattcattatgtgaatgttgaaactggggagaagacctatgtatgtgttgaatgtggaaaatcctttctcaagaagtcacagattattatacatcaaagaattcatactggagagaaaccttatgattgtggtgcatgtggaaaagcct

SEQ ID NO: 2 - protein, 131 aa, Homo sapiens
MIRSQGPVSFEDVAVDFTQEEWQQLDYAQRTLYRDVMLEIYSHLVSMGYPVSKPDVISKLEQGEEPWIIKRHIPNWIYPDRESRLDTPQLDIFRDVFFHKETLESITRGHSLYSILKVWQGDDQLERDQEN

SEQ ID NO: 3 - DNA, 341 bp, Homo sapiens
gtgaaatctgtctgcaaaagtgtggaccttactgctcgtgtagtaggcctccttgccgtcgtcttcccctcccttcgcagtcactgtggtacagtccacattccggctcagcgcctggcgagtcctccgcttgtactgagacgcctgggatctcaaaatggcggccccgtgcggaaacagcgtctgggagcagtcatgttgcctcctgaacaaagccgctgaagatgaagaatgggcaaaatcgccccatacggaacagcgcagcctcgggagcccgttacctggctcgcgaacacgaagcgggagagttcgccaatatggatgtgacagcggttcccatt

SEQ ID NO: 4 - DNA, 8765 bp, Homo sapiens
gcagaagtctgggcagctgcgggaggagaggtttgggaggcgcgggagatgtccaccctgggctggtggcgccgccgggcgccgggcgccatgagggtgcgctaggcggctgttcgtgcccgaggctgcgcagcactgaggtgagctttgccttcttgatcttccgtccttcttggagacgactggcgagaggaagagggactaggtccaaacgctaggtggctgggtccagatacctgtgttttgactctgttcctgtggatagctgcttggtctgaagttccagaaaggatcctgttcccagacaggtccctgagtactgacggtcacaggctgccgcgtctttcctgttgactcatgtttggttcctccagtgaaaattttacttagagaaatgctgcctccaaagcacttgtctgccaccaaacctaagaagtcctgggccccaaatctgtatgagctagacagtgacttgactaaggagccggatgtcatcataggagaaggtccaactgactctgagttttttcatcagaggtttcggaacctaatctatgtggaatttgttgggcctcggaagaccctgatcaaactccgaaacctctgcctcgattggttgcagccggagacccgcaccaaggaggagatcatcgagctcttggtccttgagcagtacctgaccatcatccctgaaaagctcaagccttgggtgcgagcaaaaaagccggagaactgtgagaagctcgtcactctgctggagaattacaaggagatgtaccaaccagaagacgacaacaacagtgacgtgaccagcgacgacgacatgacccggaacagaagagagtcctcaccacctcactcagtccattctttcagtgaccgggactgggaccggaggggcagaagcagagacatggagccacgagaccgctggtcccacaccaggaacccaagaagcaggatgcctccgcgggatctttcccttcctgtggtggcgaaaacaagctttgaaatggacagagaggacgacagggactccagggcttatgagtcccgatctcaggatgctgaatcataccaaaatgtggtggacctcgctgaggacaggaaacctcacaacacaatccaggacaacatggaaaactacaggaagctgctctccctcgtgcagcttgctgaagacgatggccactcccacatgacgcagggccactcatcaagatccaagagaagtgcctacccaagcaccagtcgaggtctaaaaactatgcctgaagccaaaaaatcaacccaccggcgggggatttgtgaagatgaatcttcccacggagtgataatggaaaaattcatcaaggatgtgtcacgcagttccaaatcgggaagagcaagggagtcaagcgaccggtcacagagattccccagaatgtcagatgataactggaaggacatttcattgaacaagagggagtcagtgatccagcagcgggtttatgaagggaatgcatttaggggaggctttaggtttaattcaacccttgtttccagaaagagagttcttgaaagaaagaggcgctatcattttgacacagatgggaagggctcgattcacgatcaaaaaggctgtcccaggaagaagccctttgaatgtggtagtgagatgagaaaagccatgagcgtgagcagcctgagcagcctcagctccccctcctttaccgagtcacagccaattgattttggggcaatgccatatgtatgtgatgagtgtgggaggtcgttcagtgtcatctcagaatttgttgagcaccagatcatgcatactagagagaacctctatgagtatggtgagtcctttatccacagtgtggctgtcagtgaagttcagaaaagtcaggttggagggaaacgttttgaatgtaaggactgtggagagaccttcaataagagtgccgccttggctgaacatcggaagattcatgctagaggttatcttgtggaatgtaagaatcaggaatgtgaggaagccttcatgcctagccccacctttagtgagcttcagaaaatatatggcaaagacaaattctacgagtgcagggtgtgtaaggaaaccttccttcatagttctgccctgattgagcaccagaaaatccactttggggatgacaaagataatgagcgtgaacatgaacgtgaacgtgaacgtgagcgcggggaaacctttaggcccagcccagcccttaatgagtttcagaaaatgtatggtaaagagaaaatgtacgaatgtaaggtgtgtggggagactttccttcatagctcatccctgaaagaacatcagaaaatccatactagagggaacccatttgaaaacaagggtaaagtgtgtgaggaaacctttattcctggtcagtcccttaaaaggcgtcagaaaacttacaataaggagaagctctgtgactttacagatggccgggatgccttcatgcaaagctcagagctcagtgagcatcagaaaattcattctcgaaagaacctctttgaaggcagagggtatgagaaatctgtcattcatagtgggccattcactgaatctcagaagagtcatactataacaagacctcttgaaagtgatgaggacgaaaaggcgttcaccattagctctaacccctatgaaaaccagaagattcccactaaggaaaatgtctatgaggcaaaatcatatgagaggtctgttattcatagcttagcctctgtggaagctcagaaaagtcacagtgtagcagggcccagtaaaccaaaagtaatggcagagtctaccattcagagcttcgatgctatcaaccatcagagagttcgtgctggagggaacacctctgaaggaagggaatacagtaggtctgttatccatagcttagtggcttccaaacctccaagaagtcacaatggaaatgaattggtggaatctaatgagaagggagaatcctccatttatatctcagaccttaatgataagcgacagaagattcctgccagagagaacccttgtgaagggggcagtaagaatcgcaactatgaagactctgtcatacagagtgtattccgtgccaaacctcagaaaagtgttcctggagagggatctggtgagtttaagaaggatggcgaattctctgttcccagctcaaatgtccgtgaataccagaaggctcgtgctaaaaagaaatacattgagcataggagcaatgagacctctgtaattcactctctgccttttggtgaacaaacatttcgccctcgagggatgctctatgaatgtcaggagtgtggggagtgctttgctcatagctctgacctcactgagcaccagaagattcatgatagggagaagccctctggaagcagaaactatgaatggtctgtcattcgcagcttggcccctactgaccctcaaacaagttacgcccaagagcagtatgctaaagagcaagcgcggaacaaatgtaaggacttcagacaattttttgctaccagcgaagacctcaacacaaaccagaaaatctatgaccaagagaagtctcatggcgaggagtctcaaggcgagaatactgatggggaggagacccacagcgaggagacccatggtcaggagacaattgaagaccctgtcattcaaggctcagacatggaagaccctcagaaggatgaccctgatgacaaaatctatgaatgtgaggactgtggcctgggctttgtggatctcacagacctcacagaccatcagaaagtccacagcaggaagtgcctggttgacagtcgggagtacacacattctgtaattcacacccattccatcagcgagtatcagagagattacactggagagcagctgtatgaatgtccaaagtgtggggaatcttttattcatagctcattccttttcgagcatcagagaatccatgaacaagaccagttgtattccatgaaggggtgtgatgatggttttattgccctcttgcccatgaagccacggaggaatcgtgctgcagagaggaatcctgctcttgctgggtcggccattcgatgccttttgtgtggacaaggcttcattcatagctctgcccttaatgagcatatgagacttcatagggaagatgatttactggagcagagccagatggctgaggaagctatcattccaggcttagccctcactgagtttcagagaagtcagaccgaagagagactctttgaatgtgcagtctgtggagaatctttcgtcaacccagcagaacttgcagatcacgtaactgttcataagaatgagccctatgagtacgggtcctcctatactcacacctcatttcttactgagcccctcaaaggagctataccattctatgaatgcaaggattgtggtaagtcctttattcatagcacagtcctcactaaacataaggagcttcatctggaagaagaagaagaagatgaagcagcagcagctgcagcagcagcagcccaggaagttgaagccaatgtccatgttccacaagtagttctgaggattcagggcttaaacgtagaggctgctgagccagaagtggaggctgccgagccagaagtggaggctgctgagccagaagtggaggctgctgagccaaacggagaggctgaagggccagatggagaggctgcagagcccattggagaggctggacagccaaatggagaggccgagcagccaaatggggatgctgatgagccagatggtgcaggtattgaagacccagaagaaagagctgaagagccagagggaaaagctgaagagccagagggagatgccgacgagcctgacggtgtgggaattgaagacccagaagaaggtgaagatcaagagattcaggtagaagaaccatactatgactgccatgaatgcacagaaaccttcacttccagcacagcattcagtgaacacctgaaaactcatgccagcatgatcatatttgagcctgcaaatgcctttggggagtgctcaggctacatcgaacgtgccagcaccagcacaggtggtgccaatcaagctgatgagaagtacttcaaatgtgacgtctgtgggcagctcttcaatgaccgcctgtccctcgccagacaccagaatacccacactggctgagggcatggggtaaaggttagaaaaccttcacctaggacttgacccttaccaaaccacagagaatccaaaccaatccatgataatgtcagtaggagacttaaccttagtgtgttacacacctgacttaacatctctaaactcagattgaaaagagaccgaatgtgcagattccacagtcttaagctttccccttcagatgtcagtgtctgcatgtgggaaagccatagcacacatcttacctttccaagtaatcagattgagaaaaccctatgagtattccagactacagagtttgcccaaatcaactgtaaatgacacttgtgtaacgtatatatagtgtttcatgaggtgtatataaaatagcaaattatgacagaacagtgatcacatatatttggatttatatgatatacagttacagtttactctgcagaggtaccttacctggtattctttgaattttttttttttttggaggaggaagagagcaacaaatttgattatatttttaagtgtcttagatcctgagaaagatttattgtgcattatttgaaccctgtcaatatctttttgagtaattgttttgtttcttacccttaaatagtcttgtgaagctgtaggcatgatagataacatggcttttactccttactgtttgaaaagataagtactttagcttctttctgcagccatttcatctgcgccaacactttggaacctaatactgtgtaaggctttacaatatacggattggctttttgtgacccagattgattggttgccacatgttatgtttgttgaagtggttctcatgcaaaaatattacacatttgtgttctgggttttttttttttttaaccaactcaatatgtgtttgatgatagtgaattgataaaacccgaagcttttccctgtaaatcttacatctttgcctttaaagaatgggttacaaccatcactagatcacagtagtgcctaatgaaggttgagaaccgtaggagaggctctcatgctgtaaataatgttgcaggctaataacctttcatcacttcctttgtgcgcttcctgccttaagtgacaagtagcaacatggcttgggtcccctgtgcagcatcagcttatgctgccacaagtcagtttgcaccctaggtgcccaggagctagtatccttagatctttctatcgctaacttaattctcttcgttatttatctgaccctctaactccatgtctaacttgcattaaaaaaaaaaaaattctttacagtcaacccaagcttaacatggactcaggttccccagcagccttaatttgttttgttaacatctgttccttctttttcagctctcctagagtatttctgagtgttgtgttcatctaatcttagtattcttttaattacaaattgacctcacagcttgaggtttcttgtgtcctattctgtggactacctgtgctcctttgcttcccctcccctcgcataataactatattaagaaattttttttggccttgagttggctggaaaaaaaatataaaatttaaaaaatttaaaaaaaaagatttgcaaaatgtaagtgtagatcatttgaacaagcaaaattaaagtacccactgggggaaatgtgtctgaatcttactcttctggatctgcaggattagggcttggaagtatgtcaaagatgcagggagtgtcaaagtttaggaagattgtagagctgagagcaagaagcagaaatgagtgagtcaaagaagggagtcctaatatatcaccagatctaggaggggagaggagacagacagaagaaaacaccagaggcaagaactgtagaaggccaggtttctgagaatgaattgagcggggtgtcctgagcagtttggaaaaggagtttttgatggtatggtgtaggtgagggctggctgcataggaaggactgaggttggaacggacatcgggaaagctgaggggcagtgaggtttactacatgggaaaaggactcttgaaacgagaatcagtgttgatgtcggggtgaactttgtgggtacattacttggtgttaacattgttggcagtggtagccccttttcagaaagcaacttgctgtaagtcagggtgtccgttccaaccttcagctagtgaaaaggtagtaacaaatggtaaacaagagaatgattgtttaaacctatctgtggacacttaatgcaactgtttaaaaatgataatcacgagttatgtagcaacgtggaaatatatttacagaacattaagtggagaaagcaggacacgaaagtatatttatactacagttataactcaacagttcatttatatgctgttcatttaacagttcatttaaacagttcattataactgtttaaaaatatatatgcttatagtcaaaagctgttgtggtgttgttgttgtaggcttatagttgagcattattttcttaaatttcttgaatgttctttatggtagtgttactaaaaagtttatgatcacattttcattgtgaacataatttgaactcattatcacacacttggaaaatacagaaaagtggaggaaaaaaaatcatatccccaccatccaaagacatatactctcctcttatcttgttcattcttgtttctgtgcacaggtttatgattataactgtgtcaaaatgtatattcaaaatagctgttacattacctttgtggaattatggttaaatactttcactttaattttttcaaatgttccctataataatgtcctgataacagtgtattatgtgtgtctccattggtgtgcataatacatacccagaggaaaaattagaaaataaagtaaattattttaaaaaattacctatattcccaacacctaacaactactgctaacatcttgatctgtttcctctatcttgtttcagtgcacacgcttgtgataacagtgttaaatatgtgtgcataaagtcttaaatgaaaagatgtggaaaataactaaaatagtgttgtcattgtgggaatttggttaaatattttgtctcaaattccttaaataatctttggtgttttggtaataaattttatgtatgtattttccattacaaatataatacatactcatacaaaactttggaaattcagtaaagaaaattcacacatattcccaacacccaacaacaattaactgttaacatcttgatctgtgcactagtctgtgattattagggtgttagtgataagtatgcataaatgtcaaagatgggaagaaagatgaaaaacaagaaatagttgtgtggttgttgtgggattatggttattttgtttcggtttccttgaaaggtcatcattctagtgttttggtagtccacctttactacatatatttccattatatatgaaatgtgttcattatagaaactttgaagttacagaaatgtagaagagaaactcacccatgttttcaccatccaaagagtgtggttaacatcttgatatattttcttcatcttgtttctgtgcacaggtttttggtttgttaatatggttgtggtcattctatctgtaatagtgtcaacaataaaaataaagttaaaaataaatatttaaaaaa

SEQ ID NO: 5 - protein, 1588 aa, Homo sapiens
MLPPKHLSATKPKKSWAPNLYELDSDLTKEPDVIIGEGPTDSEFFHQRFRNLIYVEFVGPRKTLIKLRNLCLDWLQPETRTKEEIIELLVLEQYLTIIPEKLKPWVRAKKPENCEKLVTLLENYKEMYQPEDDNNSDVTSDDDMTRNRRESSPPHSVHSFSDRDWDRRGRSRDMEPRDRWSHTRNPRSRMPPRDLSLPVVAKTSFEMDREDDRDSRAYESRSQDAESYQNVVDLAEDRKPHNTIQDNMENYRKLLSLVQLAEDDGHSHMTQGHSSRSKRSAYPSTSRGLKTMPEAKKSTHRRGICEDESSHGVIMEKFIKDVSRSSKSGRARESSDRSQRFPRMSDDNWKDISLNKRESVIQQRVYEGNAFRGGFRFNSTLVSRKRVLERKRRYHFDTDGKGSIHDQKGCPRKKPFECGSEMRKAMSVSSLSSLSSPSFTESQPIDFGAMPYVCDECGRSFSVISEFVEHQIMHTRENLYEYGESFIHSVAVSEVQKSQVGGKRFECKDCGETFNKSAALAEHRKIHARGYLVECKNQECEEAFMPSPTFSELQKIYGKDKFYECRVCKETFLHSSALIEHQKIHFGDDKDNEREHERERERERGETFRPSPALNEFQKMYGKEKMYECKVCGETFLHSSSLKEHQKIHTRGNPFENKGKVCEETFIPGQSLKRRQKTYNKEKLCDFTDGRDAFMQSSELSEHQKIHSRKNLFEGRGYEKSVIHSGPFTESQKSHTITRPLESDEDEKAFTISSNPYENQKIPTKENVYEAKSYERSVIHSLASVEAQKSHSVAGPSKPKVMAESTIQSFDAINHQRVRAGGNTSEGREYSRSVIHSLVASKPPRSHNGNELVESNEKGESSIYISDLNDKRQKIPARENPCEGGSKNRNYEDSVIQSVFRAKPQKSVPGEGSGEFKKDGEFSVPSSNVREYQKARAKKKYIEHRSNETSVIHSLPFGEQTFRPRGMLYECQECGECFAHSSDLTEHQKIHDREKPSGSRNYEWSVIRSLAPTDPQTSYAQEQYAKEQARNKCKDFRQFFATSEDLNTNQKIYDQEKSHGEESQGENTDGEETHSEETHGQETIEDPVIQGSDMEDPQKDDPDDKIYECEDCGLGFVDLTDLTDHQKVHSRKCLVDSREYTHSVIHTHSISEYQRDYTGEQLYECPKCGESFIHSSFLFEHQRIHEQDQLYSMKGCDDGFIALLPMKPRRNRAAERNPALAGSAIRCLLCGQGFIHSSALNEHMRLHREDDLLEQSQMAEEAIIPGLALTEFQRSQTEERLFECAVCGESFVNPAELADHVTVHKNEPYEYGSSYTHTSFLTEPLKGAIPFYECKDCGKSFIHSTVLTKHKELHLEEEEEDEAAAAAAAAAQEVEANVHVPQVVLRIQGLNVEAAEPEVEAAEPEVEAAEPEVEAAEPNGEAEGPDGEAAEPIGEAGQPNGEAEQPNGDADEPDGAGIEDPEERAEEPEGKAEEPEGDADEPDGVGIEDPEEGEDQEIQVEEPYYDCHECTETFTSSTAFSEHLKTHASMIIFEPANAFGECSGYIERASTSTGGANQADEKYFKCDVCGQLFNDRLSLARHQNTHTG

SEQ ID NO: 6 - DNA, 2141 bp, Homo sapiens
gccgacgcgtgcagacgtccttctaatcctagtcttcgtttggtccggttgcactcttcctatagcccagagggcgagagggcctgtggcctgggggaaggaggacgaggttctgcctggatcccagcagtaggacgctgtgccatttgggaacaaaggaatagtctgcctggaatccctgcagatcttggggccggaggccagtccaacccttggagcaggaagaaacgcaaagttgtcaagaaccaagtcgagctgcctcagagccggcccgcagtagctgcagactccgcccgcgacgtgtgcgcgcttctctgggccagagcgagcctgttttgtgctcgggttaagagatttgtcccagctataccatgggccgcactcgggaagctggctgcgtggccgctggtgtggttatcggggctggtgcctgctactgtgtatacagactggcttggggaagagacgagaacgagaaaatctgggacgaagacgaggagtctacggacacctcagagattggggttgagactgtgaaaggagctaaaactaacgctggggcagggtctggggccaaacttcagggtgattcagaggtcaagcctgaggtgagtttgggactcgaggattgtccgggtgtaaaagagaaggcccattcaggatcccacagcggaggtggcctagaggccaaggccaaggcccttttcaacacgctgaaggaacaggcaagtgcaaaggcaggcaaaggggctagggtgggtaccatctctgggaacaggacccttgcaccgagtttaccctgcccaggaggcaggggtggaggctgccaccccaccaggagtggatctagggccgggggcagggcaagtggaaaatccaagggaaaggcccgaagtaagagcaccagggctccagctacaacatggcctgtccggagaggcaagttcaactttccttataaaattgatgatattctgagtgctcccgacctccaaaaggtcctcaacatcctggagcgaacaaatgatccttttattcaagaagtagccttggtcactctgggtaacaatgcagcatattcatttaaccagaatgccatacgtgaattgggtggtgtcccaattattgcaaaactgataaaaacaaaagaccccataattagggaaaagacttacaatgcccttaataacttgagtgtgaacgcagaaaatcagggcaagattaagacgtacatcagtcaagtgtgtgatgacaccatggtctgtcgcttggactcagctgtgcagatggctgggctaagactgttaaccaacatgactgtgactaatcattaccaacatttgctttcctattcttttccagacttttttgctttgttattcctgggaaatcacttcaccaagatacagattatgaaactaattataaactttactgaaaatccagccatgacaagagagctggtcagttgtaaagtaccatcagaattgatttccctctttaataaagaatgggatagagagattcttcttaatatccttaccctatttgagaatataaatgacaacataaaaaatgaagggctcgcatcatccaggaaagaattcagcagaagttcactttttttcttattcaaagagtctggagtttgtgttaagaaaatcaaagcactagcaaatcacaatgatctggtggtgaaagtaaaagtcctgaaagtattaaccaaactctaatttggagtctgtcccaaacaatattgagatatttgcagttggtacgatgtgatttgtaaattctttgtttttcattgtgcgtatatggtaaagagatcttttcagctgctattttggaataatgactatcatatatcataacagtgactgatgttggttgtaatggttgggtttaggatgaaccattttaaggatgccaaatgaaatattagtatttgtacacagaaagaatttattgatttgatcttattacctagattgagattttttaatctttcctctacctaaactgacaatgaattggttatacatcatgcataagctacacttttatattagtttatatttgttattctaagacttgtgtttcatcaataaagttgtgttttaagcagcagaaaaaaaaaa

SEQ ID NO: 7 - protein, 453 aa, Homo sapiens
MGRTREAGCVAAGVVIGAGACYCVYRLAWGRDENEKIWDEDEESTDTSEIGVETVKGAKTNAGAGSGAKLQGDSEVKPEVSLGLEDCPGVKEKAHSGSHSGGGLEAKAKALFNTLKEQASAKAGKGARVGTISGNRTLAPSLPCPGGRGGGCHPTRSGSRAGGRASGKSKGKARSKSTRAPATTWPVRRGKFNFPYKIDDILSAPDLQKVLNILERTNDPFIQEVALVTLGNNAAYSFNQNAIRELGGVPIIAKLIKTKDPIIREKTYNALNNLSVNAENQGKIKTYISQVCDDTMVCRLDSAVQMAGLRLLTNMTVTNHYQHLLSYSFPDFFALLFLGNHFTKIQIMKLIINFTENPAMTRELVSCKVPSELISLFNKEWDREILLNILTLFENINDNIKNEGLASSRKEFSRSSLFFLFKESGVCVKKIKALANHNDLVVKVKVLKVLTKL

SEQ ID NO: 8 - DNA, 1273 bp, Homo sapiens
gctctctggcgcggggccccttagtccgggctttttgccatggggtctctgttccctctgtcgctgctgttttttttggcggccgcctacccgggagttgggagcgcgctgggacgccggactaagcgggcgcaaagccccaagggtagccctctcgcgccctccgggacctcagtgcccttctgggtgcgcatgagcccggagttcgtggctgtgcagccggggaagtcagtgcagctcaattgcagcaacagctgtccccagccgcagaattccagcctccgcaccccgctgcggcaaggcaagacgctcagagggccgggttgggtgtcttaccagctgctcgacgtgagggcctggagctccctcgcgcactgcctcgtgacctgcgcaggaaaaacacgctgggccacctccaggatcaccgcctacaaaccgccccacagcgtgattttggagcctccggtcttaaagggcaggaaatacactttgcgctgccacgtgacgcaggtgttcccggtgggctacttggtggtgaccctgaggcatggaagccgggtcatctattccgaaagcctggagcgcttcaccggcctggatctggccaacgtgaccttgacctacgagtttgctgctggaccccgcgacttctggcagcccgtgatctgccacgcgcgcctcaatctcgacggcctggtggtccgcaacagctcggcacccattacactgatgctcgcttggagccccgcgcccacagctttggcctccggttccatcgctgcccttgtagggatcctcctcactgtgggcgctgcgtacctatgcaagtgcctagctatgaagtcccaggcgtaaagggggatgttctatgccggctgagcgagaaaaagaggaatatgaaacaatctggggaaatggccatacatggtggctgacgcctgtaatcccagcactttgggaggccgaggcaggagaatcgcttgagcccaggagttcgagaccagcctggacaacatagtgagaccccgtctatgcaaaaaatacacaaattagcctggtgtggtggcccgcacctgtggtcccagctacccgggaggctgagttgggaggatcctttgagccctgaaagtcgaggttgcagtgagccttgatcgtgccactgcactccagcctgggggacagagcacgaccctgtctccaaaaataaaataaaaataaaaataaatattggcgggggaaccctctggaatcaataaaggcttccttaaccagc

SEQ ID NO: 9 - protein, 271 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYKPPHSVILEPPVLKGRKYTLRCHVTQVFPVGYLVVTLRHGSRVIYSESLERFTGLDLANVTLTYEFAAGPRDFWQPVICHARLNLDGLVVRNSSAPITLMLAWSPAPTALASGSIAALVGILLTVGAAYLCKCLAMKSQA

SEQ ID NO: 10 - DNA, 1145 bp, Homo sapiens
attctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 11 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 12 - DNA, 782 bp, Homo sapiens
gtccaggttccgccccggagccgacttcctcctggtcggcggctgcagcggggtgagcggcggcagcggccggggatcctggagccatggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgctgtccatggacgccttggacctcaccgacaagctggtcagcttctacctggagacctacggcgccgagctcaccgctaacgtgctgcgcgacatgggcctgcaggagatggccgggcagctgcaggcggccacgcaccagggctctggagccgcgccagctgggatccaggcccctcctcagtcggcagccaagccaggcctgcactttatagaccagcaccgggctgcgcttatcgcgagggtcacaaacgttgagtggctgctggatgctctgtacgggaaggtcctgacggatgagcagtaccaggcagtgcgggccgagcccaccaacccaagcaagatgcggaagctcttcagtttcacaccagcctggaactggacctgcaaggacttgctcctccaggccctaagggagtcccagtcctacctggtggaggacctggagcggagctgaggctccttcccagcaacactccggtcagcccctggcaatcccaccaaatcatcctgaatctgatctttttatacacaatatacgaaaagccagcttgaaaaaaaaaaa

SEQ ID NO: 13 - protein, 195 aa, Homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 14 - DNA, 1244 bp, Homo sapiens
gcggagctaagatgcggaggagggtgacgcactagctctccagttcgcccgttcctggcctgacccccaccaaggcccataccgcagtaggctcctcgggctgcccctcggttgacaatggtctccaggatggtctctaccatgctatctggcctactgttttggctggcatctggatggactccagcatttgcttacagcccccggacccctgaccgggtctcagaagcagatatccagaggctgcttcatggtgttatggagcaattgggcattgccaggccccgagtggaatatccagctcaccaggccatgaatcttgtgggcccccagagcattgaaggtggagctcatgaaggacttcagcatttgggtccttttggcaacatccccaacatcgtggcagagttgactggagacaacattcctaaggactttagtgaggatcaggggtacccagaccctccaaatccctgtcctgttggaaaaacagcagatgatggatgtctagaaaacacccctgacactgcagagttcagtcgagagttccagttgcaccagcatctctttgatccggaacatgactatccaggcttgggcaagtggaacaagaaactcctttacgagaagatgaagggaggagagagacgaaagcggaggagtgtcaatccatatctacaaggacagagactggataatgttgttgcaaagaagtctgtcccccatttttcagatgaggataaggatccagagtaaagagaagatgctagacgaaaacccacattacctgttaggcctcagcatggcttatgtgcacgtgtaaatggagtccctgtgaatgacagcatgtttcttacatagataattatggatacaaagcagctgtatgtagatagtgtattgtcttcacaccgatgattctgctttttgctaaattagaataagagcttttttgtttcttgggtttttaaaatgtgaatctgcaatgatcataaaaattaaaatgtgaatgtcaacaataaaaagcaagactatgaaaggctcagatttcttgcagtttaaaatggtgtctgaggttgtactattttggccaagtctgtagaaagctgtcatttgattttgattatgtagttcatccagcccttgggcattgttatacaccagtaaagaaggctgtactcaagaggaggagctgacacatttcacttggctgcgtcttaataaacatgaatgcaagcattggc

SEQ ID NO: 15 - protein, 212 aa, Homo sapiens
MVSRMVSTMLSGLLFWLASGWTPAFAYSPRTPDRVSEADIQRLLHGVMEQLGIARPRVEYPAHQAMNLVGPQSIEGGAHEGLQHLGPFGNIPNIVAELTGDNIPKDFSEDQGYPDPPNPCPVGKTADDGCLENTPDTAEFSREFQLHQHLFDPEHDYPGLGKWNKKLLYEKMKGGERRKRRSVNPYLQGQRLDNVVAKKSVPHFSDEDKDPE

SEQ ID NO: 16 - DNA, 2459 bp, Homo sapiens
agatgcggtcttgctgtgttgcctaggctggtctcaaactcctgctctcaagtgatcctcctgcctcagcctcctgagtacatttatatttaaagtaattattgatggctctgcctgcttaggactctgcccagccaggaaggaaacctacaggaggaagaatggctgcagggtggctgacaacctggtcacagaactcagtaaccttccaggaagtggcagtggacttttcccaggaggagtgggcattgctggaccctgctcaaaaaaatctatacaaagatgtgatgctggagaactttaggaacctggcctcagtagggtatcagctctgcagacacagtctgatctccaaggtggatcaagaacagctgaagacagatgaaagaggaattttacaaggtgactgtgcagactgggaaactcaacttaaaccaaaagatacaattgctatgcagaacattcctgggggaaaaacatccaatggcataaacacggcagaaaatcaacctggtgagcactccctggagtgtaaccattgtgggaaattcagaaagaacactcgctttatttgtacaagatattgcaagggagagaaatgctataaatatataaagtatagcaaagtcttcaaccatccctcaactcttaggagtcatgtgagcattcacattggagagaaaactcttgaatttactgattgtagaaaagctttcaatcaagagtcatccctcaggaaacacttaagaactcccacaggacagaagtttcaggagtatgagcaatgtgatatgtccttcagcctacactcttcctgctcagtacgtgagcaaatacctactggagagaaaggtgatgaatgcagtgactatggcaaaatatctccccttagtgtccacacaaaaactggtagtgtggaggagggtttggaatgtaatgaacatgagaaaactttcactgaccctttgtcccttcagaactgtgtcagaactcactctggagagatgccctatgaatgcagtgactgtgggaaagccttcatttttcagtcttcccttaagaaacacatgagatctcatactggagagaagccttatgagtgtgatcactgtggaaaatcctttagccagagctctcatctgaatgtgcacaaaagaactcacactggagagaaaccctatgactgtaaggaatgtgggaaggctttcactgttccttcatcccttcagaaacatgtgagaacccacactggagagaaaccctatgaatgcagtgactgtggaaaagccttcatcgatcagtcatcccttaagaaacacacacgctctcacactggagagaagccttatgagtgtaaccagtgtggaaagtccttcagcacaggctcttaccttattgtgcacaagagaactcacactggtgagaaaacctatgagtgtaaagaatgtgggaaggcctttaggaattcctcttgcctgagggtacacgtgagaactcacactggagagaagccttataaatgtattcagtgtgaaaaagcctttagcacaagcactaaccttataatgcacaagcgaatccacaatggccagaaactccatgaatgaaatgactcagggaagtgtttgttgcccctcatgcctcctttctcactttagaacatatattggagagaagccctgttatggtcacctggaaacagccttctggcccaactctggatgcctgttatactgggacaaaccttataaatgttatagctatgattgtttttatcagtgagtatttcattcttatatgtgtcacaactgtagatcctatgaatattttagttatcttttcagtttaattgcattgtgaacagaacacatggtcttcaatatgtagattctgtgtgacatagatttgaacataattgctggacactgactgatgtactgtggcagacagaactagttgctgtctcaatatccattcctcccttctttcttaaagtaataaaatttaccatttagttcaagctggcaatgtggacagttttcacccagcttcccttacagctgtgagcggccaaatatgttcacatccctagcccatgcaaagtaggcagaagtcactggatacagctgctaaggaagctcatggaaatgggaatatctcatctggtgtttttttcttaactatttgccctttgtcttgtcccttatttcctagaaaatatatacaccaagaggaggacagagaaaggatactaaatgaaacaacaggggagatacaatattatctggttgcactaatcctgtgctgcttatctctggatttcatgttacatgagaaaaattaactgccatgtggtttaaaccactcttgtgggaagttcctctttcagctgatttcagtcccaactgttacatctaataaatggtcaagttttaggaatcgttcaaaaaaaaaaaaaa

SEQ ID NO: 17 - protein, 481 aa, Homo sapiens
MAAGWLTTWSQNSVTFQEVAVDFSQEEWALLDPAQKNLYKDVMLENFRNLASVGYQLCRHSLISKVDQEQLKTDERGILQGDCADWETQLKPKDTIAMQNIPGGKTSNGINTAENQPGEHSLECNHCGKFRKNTRFICTRYCKGEKCYKYIKYSKVFNHPSTLRSHVSIHIGEKTLEFTDCRKAFNQESSLRKHLRTPTGQKFQEYEQCDMSFSLHSSCSVREQIPTGEKGDECSDYGKISPLSVHTKTGSVEEGLECNEHEKTFTDPLSLQNCVRTHSGEMPYECSDCGKAFIFQSSLKKHMRSHTGEKPYECDHCGKSFSQSSHLNVHKRTHTGEKPYDCKECGKAFTVPSSLQKHVRTHTGEKPYECSDCGKAFIDQSSLKKHTRSHTGEKPYECNQCGKSFSTGSYLIVHKRTHTGEKTYECKECGKAFRNSSCLRVHVRTHTGEKPYKCIQCEKAFSTSTNLIMHKRIHNGQKLHE

SEQ ID NO: 18 - DNA, 2802 bp, Homo sapiens
ccagcagcaggaggcgcgcgaggcaccacggcctggcggccgagagtcagggaggaacctcatttacataacggccgcccctctgtctcctggcgggggccggagtcccgcccctcgtccaacttgaaatctgttgggtcacgggccagtcactccgacctaggcaagcctgtggtggagctggaagagtttgtgagggcggtcccgggagcggattgggtctgggagttcccagaggcggctataagaaccgggaactgggcgcggggagctgagttgctggtagtgcccgtggtgcttggttcgaggtggccgttagttgactccgcggagttcatctccctggttttcccgtcctaacgtcgctcgcctttcagtcaggatgtctgcccgtggcccggctatcggcatcgacctgggcaccacctattcgtgcgtcggggtcttccaacatggcaaggtggagatcatcgccaacgaccagggcaatcgcaccacccccagctacgtggccttcacggacaccgagcgcctcatcggcgacgccgccaagaaccaggtggccatgaaccccaccaacaccatcttcgacgccaagaggctgattggacggaaattcgaggatgccacagtgcagtcggatatgaaacactggccgttccgggtggtgagcgagggaggcaagcccaaagtgcaagtagagtacaagggggagaccaagaccttcttcccagaggagatatcctccatggtcctcacgaagatgaaggagatcgcggaagcctacctggggggcaaggtgcacagcgcggtcataacggtcccggcctatttcaacgactcgcagcgccaggccaccaaggacgcaggcaccatcacggggctcaatgtgctgcgcatcatcaacgagcccacggcggcggccatcgcctacggcctggacaagaagggctgcgcgggcggcgagaagaacgtgctcatctttgacctgggcggtggcactttcgacgtgtccatcctgaccatcgaggatggcatcttcgaggtgaagtccacggccggcgacacccacctgggcggtgaggacttcgacaaccgcatggtgagccacctggcggaggagttcaagcgcaagcacaagaaggacattgggcccaacaagcgcgccgtgaggcggctgcgcaccgcttgcgagcgcgccaagcgcaccctgagctcgtccacgcaggcgagcatcgagatcgactcgctctacgagggcgtggacttctatacgtccatcacgcgcgcccgcttcgaggagctcaatgccgacctctttcgcgggaccctggagccggtggagaaggcgctgcgcgacgccaagctggacaagggccagatccaggagatcgtgctggtgggcggctccactcgtatccccaagatccagaagctgctgcaggatttcttcaacggcaaggagctgaacaagagcatcaaccccgacgaggcggtggcctatggcgccgcggtgcaggcggccatcctcatcggcgacaaatcagagaatgtgcaggacctgctgctactcgacgtgaccccgttgtcgctgggcatcgagacagctggcggtgtcatgaccccactcatcaagaggaacaccacgatccccaccaagcagacgcagaccttcaccacctactcggacaaccagagcagcgtactggtgcaggtatacgagggcgaacgggccatgaccaaggacaataacctgctgggcaagttcgacctgaccgggattccccctgcgcctcgcggggtcccccaaatcgaggttaccttcgacattgacgccaatggcatccttaacgttaccgccgccgacaagagcaccggtaaggaaaacaaaatcaccatcaccaatgacaaaggtcgtctgagcaaggacgacattgaccggatggtgcaggaggcggagcggtacaaatcggaagatgaggcgaatcgcgaccgagtcgcggccaaaaacgccctggagtcctatacctacaacatcaagcagacggtggaagacgagaaactgaggggcaagattagcgagcaggacaaaaacaagatcctcgacaagtgtcaggaggtgatcaactggctcgaccgaaaccagatggcagagaaagatgagtatgaacacaagcagaaagagctcgaaagagtttgcaaccccatcatcagcaaactttaccaaggtggtcctggcggcggcagcggcggcggcggttcaggagcctccgggggacccaccatcgaagaagtggactaagcttgcactcaagtcagcgtaaacctctttgcctttctctctctctctttttttttgtttgtttctttgaaatgtccttgtgccaagtacgagatctattgttggaagtctttggtatatgcaaatgaaaggagaggtgcaacaacttagtttaattataaaagttccaaagtttgttttttaaaaacattattcgaggtttctctttaatgcattttgcgtgtttgctgacttgagcatttttgattagttcgtgcatggagatttgtttgagatgagaaaccttaagtttgcacacctgttctgtagaagcttggaaacagtaaaatatataggagcttaaattgtttatttttatgtactactttaaaactaaactgaacattgcagtaatgttaaggacaggtatactttttgcaaacaaatgcataaatgcaaatgtaaagtaaagctgaaattgatctcaaaaaaaaaaaaaaaaaaaaaaaaaaaaaaaaaaa

SEQ ID NO: 19 - protein, 639 aa, Homo sapiens
MSARGPAIGIDLGTTYSCVGVFQHGKVEIIANDQGNRTTPSYVAFTDTERLIGDAAKNQVAMNPTNTIFDAKRLIGRKFEDATVQSDMKHWPFRVVSEGGKPKVQVEYKGETKTFFPEEISSMVLTKMKEIAEAYLGGKVHSAVITVPAYFNDSQRQATKDAGTITGLNVLRIINEPTAAAIAYGLDKKGCAGGEKNVLIFDLGGGTFDVSILTIEDGIFEVKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNALESYTYNIKQTVEDEKLRGKISEQDKNKILDKCQEVINWLDRNQMAEKDEYEHKQKELERVCNPIISKLYQGGPGGGSGGGGSGASGGPTIEEVD

SEQ ID NO: 20 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 21 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 22 - DNA, 8568 bp, Homo sapiens
cctcgctccctctccggtaatgagggggctgagctgtccctccgaggagggggcctggtgtggataaaagagacgaaaaagccgggggaggtttccaaaaataaaaccgtccgggtccccttcagacggctgcaggcacagggaggaggcgcgaaggtgcagcagccgtgcgagcccagctggagtaggagcgcggactcgaggctcggggcgcgcagccctcgttccgccgagagccgggcccccagtcggccgcttcagggccccctagactcagagaagctggccgccgggcggggccgggagaacagcccgcgggcgtccagcgtgccgaccacaaagctcttcgcggtgcccgcgcgcaccactctccagccgccccgcgccatgaggccgcggaccaaagcccgcagcccggggcgcgccctgcggaacccctggagaggcttcctgccgctcaccctggctctcttcgtgggcgcgggtcatgcccaaagggaccccgtagggagatacgagccggctggtggagacgcgaatcgactgcggcgccctgggggcagctacccggcagcggctgcagccaaggtgtacagtctgttccgggagcaggacgcgcctgtcgcgggcttgcagcccgtggagcgggcccagccgggctgggggagccccaggaggcccaccgaggcggaggccaggaggccgtcccgcgcgcagcagtcgcggcgtgtccagccacctgcgcagacccggagaagcactcccctgggccagcagcaaccagcaccccggacccgggccgcgccggctctcccacgcctggggaccccacagcggtctggggctgcgcccccaaccccgccgcgagggcggctcacggggaggaacgtctgcgggggacagtgctgcccaggatggacaacagcaaacagcaccaaccactgtatcaaacccgtttgcgagccgccgtgccagaaccggggctcctgcagccgcccgcagctctgtgtctgccgctctggtttccgtggagcccgctgcgaggaggtcattcccgatgaggaatttgacccccagaactccaggctggcacctcgacgctgggccgagcgttcacccaacctgcgcaggagcagtgcggctggagagggcaccttggccagagcacagccgccagcaccacagtcgccgcccgcaccacagtcgccaccagctgggaccctgagtggcctcagccagacccacccttcccagcagcacgtggggttgtcccgcactgtccgacttcacccgactgccacggccagtagccagctctcttccaacgccctgcccccgggaccaggccttgagcagagagatggcacccaacaggcggtacctctggagcacccctcatccccctgggggctgaacctcacggagaaaatcaagaagatcaagatcgtcttcactcccaccatctgcaagcagacctgtgcccgtggacactgtgccaacagctgtgagaggggcgacaccaccaccctgtacagccagggcggccatgggcacgatcccaagtctggcttccgcatctatttctgccagatcccctgcctgaacggaggccgctgcatcggcagggacgaatgctggtgccccgccaactccaccgggaagttctgccacctgcctatcccgcagccggacagggagcctccagggagggggtcccgccccagggccttgctggaagccccactgaagcagtccactttcacactgccgctctccaaccagctggcctccgtgaacccctccctggtgaaggtgcacattcaccacccacccgaggcctcagtgcagatccaccaggtggcccaggtgcggggcggggtggaggaggccctagtggagaacagcgtggagaccagacccccgccctggctgcctgccagccctggccacagcctctgggacagcaacaacatccctgctcggtctggagagccccctcggccactgcccccagcagcacccaggcctcgaggactgctgggccggtgttacctgaacactgtgaacggacagtgtgccaaccctctgctggagctgactacccaggaggactgctgtggcagtgtgggagccttctggggggtgactttgtgtgccccatgcccacccagaccagcctccccggtgattgagaatggccagctggagtgtcctcaggggtacaagagactgaacctcactcactgccaagatatcaacgagtgcttgaccctgggcctgtgcaaggacgcggagtgtgtgaataccaggggcagctacctgtgcacatgcagacctggcctcatgctggatccatcgcggagccgctgtgtgtcggacaaggcaatctccatgctgcagggactgtgctaccggtcgctggggcccggcacctgcaccctgcctttggcccagcggatcaccaagcagatatgctgctgcagccgcgtgggcaaagcatggggcagcgagtgtgagaaatgccctctgcctggcacagaggccttcagagagatctgccctgccggccacggctacacctacgcgagctccgacatccgcctgtccatgaggaaagccgaggaggaggaactggcaaggcccccaagggagcaagggcagaggagcagcggggcactgcccgggccagcagagaggcagcccctccgggtcgtcacggacacctggcttgaggccgggaccatccctgacaagggtgactctcaggctggccaggtcacgaccagtgtcactcatgcacctgcctgggtcacagggaatgccacaaccccaccaatgcctgaacaggggattgcagagatacaggaagaacaagtgaccccctccaccgatgtgctggtgaccctgagcaccccaggcattgacagatgcgctgctggagccaccaacgtctgtggccctggaacctgcgtgaacctccccgatggatacagatgtgtctgcagccctggctaccagctgcaccccagccaggcctactgcacagatgacaacgagtgtctgagggacccctgcaagggaaaagggcgctgcatcaaccgcgtggggtcctactcctgcttctgctaccctggctacactctggccacctcaggggcgacacaggagtgtcaagatatcaatgagtgtgagcagccaggggtgtgcagcggggggcagtgcaccaacaccgagggctcgtaccactgcgagtgtgatcagggctacatcatggtcaggaaaggacactgccaagatatcaacgaatgccgtcaccccggtacctgccctgatgggagatgcgtcaattcccctggctcctacacttgtctggcctgtgaggagggctaccggggccagagtgggagctgtgtagatgtgaatgagtgtctgactcccggggtctgtgcccatggaaagtgcaccaacctagaaggctccttcagatgctcttgtgagcagggctatgaggtcacctcagatgagaagggctgccaagatgtggatgagtgtgccagccgggcctcatgccccacaggcctctgcctcaacacggagggctccttcgcctgctctgcctgtgagaacgggtactgggtgaatgaagacggcactgcctgtgaagacctagatgagtgtgccttcccgggagtctgcccctccggagtctgcaccaacacggctggctccttctcctgcaaggactgcgatgggggctaccggcccagccccctgggtgactcctgtgaagatgtggatgaatgtgaagacccccagagcagctgcctgggaggcgagtgcaagaacactgtgggctcctaccagtgcctctgtccccagggcttccagctggccaatggcaccgtgtgtgaggatgtgaatgagtgcatgggggaggagcactgcgcaccccacggcgagtgcctcaacagccacgggtctttcttctgtctgtgcgcgcctggcttcgtcagcgcagaggggggcaccagctgccaggatgtggacgagtgtgccaccacagacccgtgtgtgggagggcactgtgtcaacaccgagggctccttcaactgtctatgtgagactggcttccagccctccccagagagtggagagtgtgtggatattgacgagtgtgaggactatggagacccggtgtgtggcacctggaagtgtgaaaacagccctggctcctaccgctgtgttctgggctgccagcctggcttccacatggccccgaacggagactgcattgacatagacgagtgcgccaacgacaccatgtgtggcagccacggcttctgtgacaacactgatggctccttccgctgcctctgtgaccagggcttcgagatctctccctcaggctgggactgtgtggatgtgaacgagtgtgagcttatgctggcggtatgtggggccgcgctctgtgagaacgtggagggctccttcctgtgcctctgtgccagtgacctggaggagtacgatgcccaggaggggcactgccgcccacggggggctggaggtcagagtatgtctgaggccccaacgggggaccatgccccggcccccacccgcatggactgctactccgggcagaagggccatgcgccctgctccagtgtcctgggccggaacaccacacaggctgaatgctgctgcacccagggcgctagctggggagatgcctgtgacctctgcccgtctgaggactcagctgaattcagcgagatctgccctagtggaaaaggctacattcctgtggaaggagcctggacgtttggacagaccatgtacacagatgcggatgagtgtgtgatattcgggcctggtctctgcccgaacggccggtgcctcaacaccgtgcctggttatgtctgcctgtgcaatcccggcttccactacgatgcttcccacaagaagtgtgaggatcacgatgagtgccaggacctggcctgtgagaatggcgagtgcgtcaacacggagggctccttccactgcttctgcagccccccgctcaccctggacctcagccagcagcgctgcatgaacagcaccagcagcacggaggacctccctgaccacgacatccacatggacatctgctggaaaaaagtcaccaatgatgtgtgcagcgaacccctgcgtgggcaccgcaccacctacacggaatgctgctgccaggacggcgaggcctggagccagcagtgtgctctgtgtcccccgaggagctctgaggtctatgctcagctgtgcaacgtggctcgcattgaggcagagcgggaggccggggtccacttccggccaggctatgagtatggccccgggcccgatgacctgcactacagcatctatggcccagatggggcccccttctacaactacctgggccccgaggacaccgtccctgagcctgccttccccaacacagccggtcactcagcggaccgcacacccatccttgagtctcctttgcagccctcagaactccagccccactacgtggccagccatccagagcccccagccggcttcgaagggcttcaggcggaggagtgcggcatcctgaacggctgtgagaatggccgctgtgtgcgcgtgcgggagggctacacctgtgactgttttgagggcttccagctggatgcggcccacatggcctgcgtagatgtgaatgagtgtgatgacttgaacgggcctgctgtgctctgtgtccatggttactgcgagaacacagagggctcctaccgctgccactgctccccgggatatgtggctgaggcagggcccccccactgcactgccaaggagtagcagtcaggggtcagtgtggcaactacctggaaatggcctccagtcacaggcaggggccttgaggatgatttcctagctgggaagacaccgtgacatcaggccagaggtttccaatcagccttgcctgctttcatctctcccagcttagcctctggctgtaagcttcggtcattgcctccatgcccttgcttggctcaagcaccaccaatcgctttaatgcttcagccaccgcatgaggccctgtccaccacctttcctggccttgctatgggatgcttaccaaaggatggccctcatccaccctcccaagctgtgcgagcatgcaaggccccatggcctcacactgcagacacccctttccagccacaatccaccatcatcctgacgatcccacaactgggacagaggctacatctgccctagggaggtccttcagaatctgtggagcaagaaaggatttggggaagcttggggactgactccagagccccctcctaagaaccatcaccaccactcagccaatctgttctgggccctgattttgccacacctccatcctgtagcccattctctgaccccaaggagtggcagaagatcccttcactcagagaagcaaggctgatattagcttgttgaatgtaagagacacaaatgaagaagaacaaagagcctgagaaagcagcaagaggacatgatgaaaaatacgtggagttgatgagaaaggggagccaaggctttatacgtctaaagaaaatattcagtagctgaatccgcccagtgatagcctgtgggcaccagcagcaagggctgccatgggatacagcacccatctacaaagacctctattacataaacactgcttcttacaggaaacaaacctcttctgggatctccttttgtgaaaaccagtttgatgtgctaaaagtaaaaagtctattttccagtgtggtcttgttcagaagcagccagatttccaatgttgtttttcccctccactcagaaacccctgccctttcccttcagaaaacgatggcaggcattcctctgagtttacaagcagagactcactccaacccaaactagctgggagttcagaaccatggtggaataaagaaatgtgcatctggtctcttctgttgtttttatttcatatcagattaaatttctttaccatgttggctaagtctaaatattagagatgaggctgtgcctactccctggccagctctgctgatagcctatgatgggttccaatgggaaatgactctttactattaaaagacaaggaaagctctgacttcgtacttctctgatgaatggcaatgtaaatgaacaaggctccatgtgactggagcatggaagtgaatgctactttcttaatttaatctgccctgtcctacctgctcctctgattgttagccatcacataacttattgaatgcttgccatgtgccaggcactgtgctgagtgccatacatacatttcatttaattatccaataatcctacttactattgtttattctcaatttacaggtgaggaaactgagacatagaaagcttaaataatttgctctaggtccctatactaattgagatgttttcagcagacaaaaataagtcactcttacgcatgtaaaaatattctttcactcataataaaagaaatgcaacttaaaaccatactgagatcctttttaaaactgtcagattggcaaagatcaaaaagcttgttaagactcctagctggcaagcatgtgggaaataggtactctcataacagctgacagaagtacatattgaagaggacaatatggcaatgtccttcaaaattataaaagcactaactctttgatccagtagtttccacttctaggaacttaaccttcagatatattttcaccagatataaaatgacatacgtaagaagttattactgcatcatttgtaatagctaaagattagaaacaattaattgctatggaacccttaaaaaaggtggctctaaattccttaagaaagatatccaaaacaaattaaggggaaaagggagggttgcataacagcatgtaacataacactatttgtctaaagaatggggaaagagaagcggttgtctgttcacctcatccttgccactccgccttatacctcaccatcggagaacttaccatcccccaaatatatcctgctggcactacaccttcgtaactgctgttcccactgcccccacaacactccaatctccaccttcctgccaaccttgagatattgtatgcatatatacatatttcctttgtttaaaatacaaatagcatactatgcatgctgttctgcactttgctttttttttatacttgcaattagagctgcctcatttcattttacagctgtatactattccattgaatggataaaccataatttatttaatcagtccctcactgacagacgtttccattgtttcctgtcttttgctataaataatgctgtaatgaatattcttgtgcatatttcacattaatttatgggattacatctgcaggataaattcttagaaccaatggtggtttgtggggagggtgacagggtggataggtacaggattgggagacttttcactgtatctccttttgtatttttttgaattttgtaccatatgtatcacctattcaaaaaaaataaaatatttttaaatacaaaaaaaaaaaaaaa

SEQ ID NO: 23 - protein, 1821 aa, Homo sapiens
MRPRTKARSPGRALRNPWRGFLPLTLALFVGAGHAQRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGASWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGEAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 24 - DNA, 5849 bp, Homo sapiens
gcgttccttgcggcccggccgacctcgcgggcttgggcctgggcgggcaccgacggagcggccctggctgcagcctcccggcgccagcgaagacaggctgagatgcggctgctcctgctcgtgccgctgctgctggctccagcgcccgggtcctcggctcccaaggtgaggcggcagagtgacacctggggaccctggagccagtggagcccctgcagccggacctgtggagggggtgtcagcttccgggagcgcccctgctactcccagaggagagatggaggctccagctgcgtgggccccgcccggagccaccgctcttgtcgcacggagagctgccccgacggcgcccgggacttccgggccgagcagtgcgcggagttcgacggagcggagttccaggggcggcggtatcggtggctgccctactacagcgccccaaacaagtgtgaactgaactgcattcccaagggggagaacttctactacaagcacagggaggctgtggttgatgggacgccctgcgagcctggcaagagggatgtctgtgtggatggcagctgccgggttgtcggctgtgatcacgagctggactcgtccaagcaggaggacaagtgtctgcggtgtgggggtgacggcacgacctgctaccccgtcgcaggcacctttgacgctaatgacctcagccgagctgtgaagaatgttcgtggggaatactacctcaatgggcactggaccatcgaggcggcccgggccctgccagcagccagcaccatcctgcattacgagcggggtgctgagggggacctggcccctgagcgactccatgcccggggccccacctcggagcccctggtcatcgagctcatcagccaggagcccaaccccggtgtgcactatgagtaccacctgcccctgcgccgccccagccccggcttcagctggagccacggctcatggagtgactgcagcgcggagtgtggcggaggtcaccagtcccgcctggtgttctgcaccatcgaccatgaggcctaccccgaccacatgtgccagcgccagccacggccagctgaccggcgttcctgcaatcttcacccttgcccggagaccaagcgctggaaggcagggccatgggcaccctgctcagcctcctgtggaggaggctcccagtcccgctccgtgtactgcatctcgtctgacggggccggcatccaggaggccgtggaggaggctgagtgtgccgggctgcctgggaagccccctgccattcaggcctgtaacctgcagcgctgtgcagcctggagcccggagccctggggagagtgttctgtcagttgtggcgttggcgtccggaagcggagcgttacttgccggggtgaaaggggttctttgctccataccgcagcgtgctccttggaagaccggccacctctgactgagccctgtgtgcatgaggactgccccctcctcagtgaccaggcctggcatgttggcacctggggtctatgctccaagagctgcagctcgggcactcggaggcgacaggtcatctgtgccattgggccgcccagccactgcgggagcctgcagcactccaagcctgtggatgtggagccttgtaacacgcagccctgtcatctcccccaggaggtccccagcatgcaggatgtgcacacccctgccagcaacccctggatgccgttgggccctcaggagtcccctgcctcagactccagaggccagtggtgggcagcccaggaacacccctcagccaggggtgaccacaggggagaacgaggtgaccccaggggcgaccaaggcacccacctgtcagccctgggccccgctccctctctgcagcagcccccataccagcaacccctgcggtcgggctcagggccccacgactgcagacacagtcctcacgggtgctgccccgatggccacacggcatctctcgggcctcagtggcaaggctgccctggggccccctgtcagcagagcaggtacgggtgctgccctgacagggtatctgtcgctgaggggccccatcacgctggctgcacaaagtcgtatggtggtgacagcaccgggggcatgcccaggtcaagggcagtggcttctacagtccacaacacccaccagccccaggcccagcagaatgagcccagtgagtgccggggctcccagtttggctgttgctatgacaacgtggccactgcagccggtcctcttggggaaggctgtgtgggccagcccagccatgcctaccccgtgcggtgcctgctgcccagtgcccatggctcttgcgcagactgggctgcccgctggtacttcgttgcctctgtgggccaatgtaaccgcttctggtatggcggctgccatggcaatgccaataactttgcctcggagcaagagtgcatgagcagctgccagggatctctccatgggccccgtcgtccccagcctggggcttctggaaggagcacccacacggatggtggcggcagcagtcctgcaggcgagcaggaacccagccagcacaggacaggggccgcggtgcagagaaagccctggccttctggtggtctctggcggcaagaccaacagcctgggccaggggaggccccccacacccaggcctttggagaatggccatgggggcaggagcttgggtccagggcccctggactgggtggagatgccggatcaccagcgccacccttccacagctcctcctacaggattagcttggcaggtgtggagccctcgttggtgcaggcagccctggggcagttggtgcggctctcctgctcagacgacactgccccggaatcccaggctgcctggcagaaagatggccagcccatctcctctgacaggcacaggctgcagttcgacggatccctgatcatccaccccctgcaggcagaggacgcgggcacctacagctgtggcagcacccggccaggccgcgactcccagaagatccaacttcgcatcatagggggtgacatggccgtgctgtctgaggctgagctgagccgcttccctcagcccagggacccagctcaggactttggccaagcgggggctgctgggcccctgggggccatcccctcttcacacccacagcctgcaaacaggctgcgtttggaccagaaccagccccgggtggtggatgccagtccaggccagcggatccggatgacctgccgtgccgaaggcttcccgcccccagccatcgagtggcagagagatgggcagcctgtctcttctcccagacaccagctgcagcctgatggctccctggtcattagccgagtggctgtagaagatggcggcttctacacctgtgtcgctttcaatgggcaggaccgagaccagcgatgggtccagctcagagttctgggggagctgacaatctcaggactgccccctactgtgacagtgccagagggtgatacggccaggctattgtgtgtggtagcaggagaaagtgtgaacatcaggtggtccaggaacgggctacctgtgcaggctgatggccaccgtgtccaccagtccccagatggcacgctgctcatttacaacttgcgggccagggatgagggctcctacacgtgcagtgcctaccaggggagccaggcagtcagccgcagcaccgaggtgaaggtggtctcaccagcacccaccgcccagcccagggaccctggcagggactgcgtcgaccagccagagctggccaactgtgatttgatcctgcaggcccagctttgtggcaatgagtattactccagcttctgctgtgccagctgttcacgtttccagcctcacgctcagcccatctggcagtagggatgaaggctagttccagccccagtccaaaatagttcatagggctagggagaaaggaagatggactcttggcttcctctctctggctggcaaagggagttatcttctggaatacattagctctttcaaaaacccacccagtgtttagcctcaacggcagccagttaccagcttctctctgtagccttcagcagtgtttgcatctctgacataaccacaggctgctgttttcaagaagagcaatctgtttggataagaaaaacctttactttacagcttccctttataatttgttacacaggaatagttaaatgcatttgtttgtttgttttttgagacagagtttcactcttgttgcccaggctggagggcaatggcgcgatctcagctcactgcaacctccgtctcctgggttcttgattctcctgtgtcagccttctgagtagctaggattacagatgcctatcaccatgcctgggtaatttttgtatttttagttgagatggggtttcaccatgttggccaggctggtctcgaacttctgacctcagatgatctgcccgcctcagcctcccaaagtgctgggattacaggcatgagccaccacgcccagccatcaatgcattttttttattttttttttgagacagagtttcgcacttcttgcccaggctggagtacaatggtgcgatcttggctcactgcaacctccacctcctgggttcaagcgcttctccagcctcagcctcctgagtagctgggattacaggtatgtgccaccatgcctggctaattttgtatttttagtagagacggggtttctccatgttggtcagactggtcttgaactcccgacctcaggtaatccgcccgcctcggcctcccaaaatgctgggattagaggtgtgagccactgtgcccagcccatcaatgtgttttaaagctagctgtcagggttccacttaatttaaagctgggcagggagatgtgtaatgatttcaaagttaacacctgtttgttttctaaagggcatgccaagtcctgctgtatcagggaagtattctgtgctaaaatcagcgatggttcattgctctagtctctctcacccttctaggcagtgcatcagtcagctctaaatctggtgcagagggttaacagcataacccttgttggcaaaatggaatagatgttaagacctcaaatagggatttgggatgaaacagctgcagttagcactgttatctgagcatgaaagaactggaaacgctccttacgtcgagatgttggaccttgaagccctcctgaggccaacatgcaaatctggctgtgacggttcatctgacacctgtgtaaagctgaccagcctgctctgtacagtgacaatgaggagcccctctcttccttaagtaggaatctgtgaagcaaaatgtttgctgccaaagacaaatcagactgtcagtcattaaaaacagcattagcaggatgaggatagcaatggggaagggttgtgggcaatgcagtaacagggaaatggcttcagaaatggtttgagttggaagacaacattcttcatctctcaggacttctaattccttgatgctaaaagaagaggcatggattctatgagcttccaagtccctttccactttaaccttctacaaatctttcagaggactgcctagtagcaaaggttattcctggacacaggaaagacgggcattacagggaccaaagctctgaaaggtgacttttattaccaacacactggctggaaaagggacaaaccacatcacgggtgagtgatacttctcagtcttctctactcattcaacaaaggaaatgtgggctggggcagaggtcttttttcatttaatactggaaaaatattgaagagcatccatgttcacttatggctggttttgctatagaaattggaaaataaaggccacttttttgaaatccccagtttaattaaaaaaaaaaaaaaaaaa

SEQ ID NO: 25 - protein, 1251 aa, Homo sapiens
MRLLLLVPLLLAPAPGSSAPKVRRQSDTWGPWSQWSPCSRTCGGGVSFRERPCYSQRRDGGSSCVGPARSHRSCRTESCPDGARDFRAEQCAEFDGAEFQGRRYRWLPYYSAPNKCELNCIPKGENFYYKHREAVVDGTPCEPGKRDVCVDGSCRVVGCDHELDSSKQEDKCLRCGGDGTTCYPVAGTFDANDLSRAVKNVRGEYYLNGHWTIEAARALPAASTILHYERGAEGDLAPERLHARGPTSEPLVIELISQEPNPGVHYEYHLPLRRPSPGFSWSHGSWSDCSAECGGGHQSRLVFCTIDHEAYPDHMCQRQPRPADRRSCNLHPCPETKRWKAGPWAPCSASCGGGSQSRSVYCISSDGAGIQEAVEEAECAGLPGKPPAIQACNLQRCAAWSPEPWGECSVSCGVGVRKRSVTCRGERGSLLHTAACSLEDRPPLTEPCVHEDCPLLSDQAWHVGTWGLCSKSCSSGTRRRQVICAIGPPSHCGSLQHSKPVDVEPCNTQPCHLPQEVPSMQDVHTPASNPWMPLGPQESPASDSRGQWWAAQEHPSARGDHRGERGDPRGDQGTHLSALGPAPSLQQPPYQQPLRSGSGPHDCRHSPHGCCPDGHTASLGPQWQGCPGAPCQQSRYGCCPDRVSVAEGPHHAGCTKSYGGDSTGGMPRSRAVASTVHNTHQPQAQQNEPSECRGSQFGCCYDNVATAAGPLGEGCVGQPSHAYPVRCLLPSAHGSCADWAARWYFVASVGQCNRFWYGGCHGNANNFASEQECMSSCQGSLHGPRRPQPGASGRSTHTDGGGSSPAGEQEPSQHRTGAAVQRKPWPSGGLWRQDQQPGPGEAPHTQAFGEWPWGQELGSRAPGLGGDAGSPAPPFHSSSYRISLAGVEPSLVQAALGQLVRLSCSDDTAPESQAAWQKDGQPISSDRHRLQFDGSLIIHPLQAEDAGTYSCGSTRPGRDSQKIQLRIIGGDMAVLSEAELSRFPQPRDPAQDFGQAGAAGPLGAIPSSHPQPANRLRLDQNQPRVVDASPGQRIRMTCRAEGFPPPAIEWQRDGQPVSSPRHQLQPDGSLVISRVAVEDGGFYTCVAFNGQDRDQRWVQLRVLGELTISGLPPTVTVPEGDTARLLCVVAGESVNIRWSRNGLPVQADGHRVHQSPDGTLLIYNLRARDEGSYTCSAYQGSQAVSRSTEVKVVSPAPTAQPRDPGRDCVDQPELANCDLILQAQLCGNEYYSSFCCASCSRFQPHAQPIWQ

SEQ ID NO: 26 - DNA, 3106 bp, Homo sapiens
cagcgccagctccgcgtcccgaccggcccgcggcagcctgcgccgcgccatggccacctccccgcagaagtcgccttctgtccccaagtctcccactcccaagtcgcccccgtcccgcaagaaagatgattccttcttggggaaactcggagggaccctggcccggaggaagaaagccaaggaggtgtccgagctgcaggaggagggaatgaacgccatcaacctgcccctcagcccaattccctttgagctggaccccgaggacacgatgctggaggagaatgaggtgcgaacaatggtggatccaaactcacgcagtgaccccaagcttcaagaactgatgaaggtattaattgactggattaatgatgtgttggttggagaaagaatcattgtgaaagacctagctgaagatttgtatgatggacaagtcctgcagaagcttttcgagaaactggagagtgagaagctaaatgtggctgaggtcacccagtcagagattgctcagaagcaaaaactgcagactgtcctggagaagatcaatgaaaccctgaaacttcctcccaggagcatcaagtggaatgtggattctgttcatgccaagagcctggtggccatcttacacctgctcgttgctctgtctcagtatttccgcgcaccaattcgactcccagaccatgtttccatccaagtggttgtggtccagaaacgagaaggaatcctccagtctcggcaaatccaagaggaaataactggtaacacagaggctctttccgggaggcatgaacgtgatgcctttgacaccttgttcgaccatgccccagacaagctgaatgtggtgaaaaagacactcatcactttcgtgaacaagcacctgaataaactgaacctggaggtcacagaactggaaacccagtttgcagatggggtgtacctggtgctgctcatggggctcctggagggctactttgtgcccctgcacagcttcttcctgaccccggacagctttgaacagaaggtcttgaatgtctcctttgcctttgagctcatgcaagatggagggttggaaaagccaaaaccgcggccagaagacatagtcaactgtgacctgaaatctacactacgagtgttgtacaacctcttcaccaagtaccgtaacgtggagtgaggggctgccctgggcccaccactgcccaagagttcttgctgttggcgtactggaccctcctccgaactgccttaccctgcttattcctgtctcttgcactgtgctctcccacaagtccagctgcaacccagagatagtggaaactgaaattaggaaggaaatcatcaataactcagtgggctgacccatccctcccaggcgctggggaccaacctagcaatgaaggttgggaaggttgttcccttcccggtgccaggtccagatttccctccatgatttgggaaccagcttaggcaaaagagtccccacaagatgaaaataaagatcctagttaccattcaaaggatgctaactgtgtgtcaggccccacactaagtgctctgctctgatatactcaaggccattaatcttcaggactcccattgacgtaggtgtttcattccccttttacagatgaggaaactaaggcttggaggttaaatgacttgccagaagttggaatttttttcctctttgaacataacctctcccttctccctaaaggtaaccactattctgagtccaatcatcaaggttttgcttttctttttagctaagtatgcattcctcaatagtagacagtacaacatgtttataacaagccaattacattatgttctttgcatgttctaaagttgtgtatgtgtgtgcacatctgagcacgtgcacatgtacacctgagccaaaaacacgagaacccactgatctcaccactggggcaagctaggtcagagcttagtgattcacactgaaattggcaaattggatttaacccaattaatagtgtgtgtgtggcaggagtcatgtccctcacatcctttgtacaaatgaaaattactcttaattccttcagatttataataactctgtactttggtttcagggtgacatttgggaaggattttgtttagaattaatggagtggcacattttgcagcctttttgcttgattgcatgtaatggaaatgccctatattttcctgcaaaataagtactaaattcattatcgttaagcaaatgtacaatatgctcaggcaccgcagagagctgggcacgggcccatgtgagcatcactttggaagtagggctcttcaacagggacccttgaactttaaagaaaggaacttctttttgccttctaattgatcatttagactattctggctaagtctgcccacatgtaattaccggctaattcaagcgaggaaaaatgtaagtcatttagaccaaagccaagcagtttctttgcgtgggttactcaagggcttgtggttacttgtatctcctctatgtgaacttgactttgaaagacagagctctagtgtgccagcctgctaagtcctgtaagaatagggaagggcggaggggggtgggcagtgactaggggacgagaagcatggggaaaatatttgcactctaaacatacagagatagaggtggggcttgtggtacttaacacttgtagccatcaactgactgagaccttgggctaaataatcaattgtgctgatattacatctcgttatggaatgttcctaaatatgccaggtagacaccagcccaagtaccctcctccagaagtctgtgactaccttgtcactactttaggcccattccacaaagcccatctctggtttgagaattcattttgatctgtatctacaccacccaaagttaggcctcctataatgtccaaaacattcctttcagcctttttatttcttactgtactgtctcttactgtactgtctatctgcagtaattgaggacccataaaatttagataactacatgtctttgctcttagaattgtcactcagcataatgagcatttaacatacaaaggcaatgtactgttttgtgttgatctatgtaaaagaatacaattcttttttacat

SEQ ID NO: 27 - protein, 372 aa, Homo sapiens
MATSPQKSPSVPKSPTPKSPPSRKKDDSFLGKLGGTLARRKKAKEVSELQEEGMNAINLPLSPIPFELDPEDTMLEENEVRTMVDPNSRSDPKLQELMKVLIDWINDVLVGERIIVKDLAEDLYDGQVLQKLFEKLESEKLNVAEVTQSEIAQKQKLQTVLEKINETLKLPPRSIKWNVDSVHAKSLVAILHLLVALSQYFRAPIRLPDHVSIQVVVVQKREGILQSRQIQEEITGNTEALSGRHERDAFDTLFDHAPDKLNVVKKTLITFVNKHLNKLNLEVTELETQFADGVYLVLLMGLLEGYFVPLHSFFLTPDSFEQKVLNVSFAFELMQDGGLEKPKPRPEDIVNCDLKSTLRVLYNLFTKYRNVE

SEQ ID NO: 28 - DNA, 7759 bp, Homo sapiens
accagaggtttcccagagaggaaggcgtggctccctcccgggccagtgagccctggcgccgccgcggccgcggtcccagcagcggagtagggcggcggctgcgccccgcaccatggggggcagcccagccccagccgcggtaaacgccgacctccgccgccgcccgcgccgcgtctgccccctcccgctgcggctctctggacgccatcccctcctcacctcgaagccaacatgaaggagacccggggctacggaggggatgcccccttctgcacccgcctcaaccactcctacacaggcatgtgggcgcccgagcgttccgccgaggcgcggggcaacctcacgcgccctccagggtctggcgaggattgcggatcggtgtccgtggccttcccgatcaccatgctgctcactggtttcgtgggcaacgcactggccatgctgctcgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggccgtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctgataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccaggtagcaaatgctgtctccagctgctctaatgatggacagaaagggcagcctatctcattatctaatgaaataatacagacagaagcatgaaagaaaacacttaacttgcatgtgcacagcttttggtaacaaatatcgctaaaccttactgtgaatttaggcatctctggcatgccactgtttatgcattgaagtggaatttttggtataaagctaaatggtcttagaagcatagaaaatccctatgtgccaaaagtagtgaaacacaaacaaaggaaaatatattaataacagtctagtgtttttgttgagtctgccattcgtagctgaatatgtgattaattatgtgatgaaaacattttttataaatgatcttggtctattggggagcggggatagttaatattccagtacactgaatacatgaggaatttaaccacatacatcattgaagacaagggatagcagtttgtttttattcaaagacattgctgtgttctctttcattgcctctctcgctttctgtcacttttttcctccttacattaaagaaaagtttaattacagttaaaaatgtataatgtatttataatattcatcgataccattattcaaatattgctcaatacagcaaattagctcctaacctaacaaagtttaagtttacttggattgataattaggtttactctttatctgaataagaaccaattccatttgtttgaaatatggagtttgtgactacccaaattgctaattattctttcttttgaatatattttacatttctatgagcctaaggaagattcatgaaactgacctatgagagtcgtgaagtggtttttcagaatgctatgtaaggaccgatttgagcactaactataggtactctgaatatatatttcccttgattattcaccaaaagtgttccccagtctttgactctttaaattccaatactgattccaaaacaaataaatattttgaagactcaatgaatactttccatattttggcctatttatataagaaagttaataacattgacccttcacagctcttctgcctgctcctcaaagtggctctatctaaatatttattactaaaatgtttttcctacagtctacatgaatacaaacctcaatagctaagcttgacgtatttgtgcacaagtagatcactacattaagttttgggaattgcacttcttaaaaatgtctccccaccaaacatagtaatcctgtagttatgcctacacaaagcttgccatattctttggtgcattcattttgtaaacccattaactttttattgtgaagattttcatttgcagtttcttgcactgcttttctagttttttaaaagcttgagatttatttatacttcttgtagtaactgcatatttctgtgtgtgtttagtggtaaagaattaattttgataggtacaatatgtctatcagattgatatatacaccagcctatgtcaattggggctaattattttaaatgaccatgtcaaattgaatttggagacaaaatctgttgagagtgcttatgtaattaatgatggttctactaatctaaattttggaaaaggtgataaatagactatactaaaatctctctatgccatagaattggattatcctgtaggtcatctcattgggtataagacaaaactacctactttttttcaaaagtgcactgaaatcacataataaagaggctttacctcttggttggtcctgtgaccctaagttctagtcagatagacacagaggcaatgtgaatttgagtggcatgagcatgattaggttattccttccagcatctagtatagcacctggaatatagaaactgtctaatacatatttattcagtgaatcaatgagcagaagtttgccaggacagtacacattggcaaggcacataccatatgattgaagtgcttcatgccattacagtccatcaggctgataaagtgaattatttctgattatttaattacagaaatatgaatttatcttcaagggtgtagtgtcatactgctgtacaacacagtgctttatttatactaataatttaggagactgatacttccaaatgatagtggacattactatcacaagaatatcacttttcatcaaactgcaaaaatacagaaaggcaaaaaacctgacacttattcttaactgcaaattaaattcctgcccaggggatatattttaggaggggatgaatggcagcttttgtgttttttttaacaagcttgaaagggaggtggaaaacaaagaaattatgtaaatggcatatgagttttattatctaggcattcgttagtatggggaaacctgataagaaatgaaaatcccaaatgatttcagccttttcatgatggttgaggttagatttcagagatgtacagagactagagcggtggttagaaagaggatatatgtagtcacagcagaaagacgtgtctaagtttaattttattggctttcaagttcactcatgtatacttagtttgtccatacatatgtctaatcaggaaaaatgcatgtatagattatgacaattcctgaattttgaagtattggttaaaagacaattaaaggccaagaaaaccatggtggaagaagtaagcgaatgaaatgtagaaatatatgtaaaattagcaagtgtcaattttaccaagtagtgttgattttccaaacaatgaatttatatactatgctgagtcacagagaagaatgatcacatgttacttaatgagagcagtttacttttcaaataaaataggtatgatgaatgtcttaaaaatatcttgaagttgaagaaacaaaaatgagttatctcaatatttaccaagttaacctagtgctgtatatatcccaagatattttaggtaaatgtaagtgtttaatcatgccagatttaaactagtctgaaatatagggtatacatatatttctacttacatttctttattttatgaaatatccgaccatgttgcagaaaataatgcaaaacctcatgtaagttaactatgaaagatcctgtgagcacattggcattgagtgacagacaaactaaaaactggcaaacagtattttaataagggggtcactctgtggcagtattctaatattggattttcaagtagattaggctttttatttattcaacgctttttataattttgttctttttgactccaaattattggtcagctttcaaccttctccacatcagcaatcactaatagttcttttggttgagatcaactcagaaaaagaaaatagaaaacttttgttctttgaaattttagacatgcataatatctatttattttcataatttaaccccaaaagcttctcctgcaatacacaggattctaggagctgaatgacacagggagactacagagtatttattattacaaacacataaaaagcctaacttgaagaattaaaatttctattttttatctgtataacaagtacaaaccatcaacaatgacaaattttcacagctgcttgtttattgcttgttttatatgtttacatatctcaaaatctgttaaaactgaggtctaaaaaatgtgcagaattgtgcaactgtggcctagtccataagacttttctgagttgcaacaaagtgctgacaaagtgacagatgtctgtgatgtttcatgacataggttatgatcgagccaatttacaaaatttaataaccaacctaaactcacatacatatttgttaagaagctgacatcagttcagtattgtaaggaaactaactaggtggtgatgatgataaaagaattagggaaaatattttatttgatatattcccttttataattcctaaaatgaagattctatttaagggttataatttatataagtttagtcatataccattacattatgataccataagcagagtgcattatgattctctagaaatataattcaatcagatatgtattatatttatttatgtcacacattttctttactgagaataaaaattatcttattttcagaagctttgtatcaatcagtttcatgtaataagcaacccagatatctactagattatgtatttcttcatttgaaactacactgttttctcctagtccccttgactctactgtgcttatccattctttcacagaaagaaagtaacagacataattcctgttgatgaggctgggattgtttttaagaggagagataataacttcatatttttaaagtgccagtagcctaatatgtgaaacagatcagaatctgttgtgtagtaagtctgctttgttgaagaatttattatgggagtaaagataagaaggaaagagatcaccatcagaaacaagtcagccttttcatgcttttttgagcatttttggagatgattccacttctcaagttattatcatttgtgcatctcttcaatgctattgttaaatgctttagaattagaatattttgatcctttaattaaagtaagccaaacgtctaggcaaaaacagccaatcattaaactttaatagtaattcaaatatagatttctcatacagttttccatgtctgtagaaatcaaagttgtaatgttaagcagagggaaatgcgtgtgatttactaatacacttcaacgttctacttttgaaaggatactcatgtgggtggggcagagaacatagaaaaagatatgatggaaaacctgtccattttctacctgttaaccttcatcattttgtgccaggccctggaagcaaagagaggaagggaccgactgcatttatctttgaacacttgagcatcagtagtactactgagtggccaggggtcttgtctgtcaaagcaaatgataagttcactcaggccattattgactgctgaactctcttccttcccaactcttccttgaaagagaaaaaaatactttgccttcttgctctccttatcaaatgtttttgtacaaatagtgtaagcctgtttaagcaaaccaattaaaataggcactgattattttgatctgtttgtaacaaatgaatgtaagtactatttacatggtgtgcctaggaggagctgaaatcattggcactttaatccatattgtaaagatcagtatcaaaagcatagtgttcttcacctctcctcctcagcatccatctctatatacttgattaaatggaaaagtctcttttatcacctctatgtaaagttttatgggtagttatcgtcagtgtatttaaatatatcttctagtatgttttaaaggctggtcttcaatactgtggagacaaaaaataaaagagcgtatgaaaagtacgttagacttttgctggcattcaagtcatggctagtctgtgtatttaataaatgtgtgttatttatgtcgtgtttgtcaatggaaaataaagttgaatattctgaaatgtcgctgtgttttcttcctgatgtcaactcacgtcaggaatactttacctataactatgttaagtattttgctgaaatcccatttgatgtgctttgtcaaataatagcacaatgtaatgcaacagccccatgctatctggaaaaattagatcattttgttttatattccataatcatgtgcataacaaattattgttgatttaatataaatatgagagttgctcttttccgattttcaacatgatgtttctttgttttattatctcaagattattgttcttaacacagagtgcatttataaattattgtaagcttagttatttccctttcttgtggattttggtgtaatttacaggcagccaaaataagaattgtccttgctcatagtacaaccctttaattgacttgtgcctgagttttggtgtgagctgagtattggtggttatgaatactgggatccccagctatttgttcatgtacttattgaggtcactattggtcttccttcttcaatgaagggagaagctctgtcaaatgtagactgcaaagtcaagctcattaaaagtatccctctttcttcgtgagaaaagatatccaaggagaattctgtccctggttcctcccatgtgggggttctaggcctaggaccagatcttattgtctctacaatcttgattctttagtatttcaggataggcagagttttgttaaagtatacaaaaatcatagctagataagagagataacagaaataagttccagtgttctatactcctgtaggatgactacagttaatgctaatatacagttacaaatagctagaaggaggctattgaatgttcccaacacaaagaaatgataaatgtttgagatgatgatgtgctaattaccctcatctgaccagtatacattatatgtatcgaaacatcgctctgtgccctgcgaatatgtacaattgtgattattaaaataaatacataaataaaatttaaaagtcattatga

SEQ ID NO: 29 - protein, 388 aa, Homo sapiens
MKETRGYGGDAPFCTRLNHSYTGMWAPERSAEARGNLTRPPGSGEDCGSVSVAFPITMLLTGFVGNALAMLLVSRSYRRRESKRKKSFLLCIGWLALTDLVGQLLTTPVVIVVYLSKQRWEHIDPSGRLCTFFGLTMTVFGLSSLFIASAMAVERALAIRAPHWYASHMKTRATRAVLLGVWLAVLAFALLPVLGVGQYTVQWPGTWCFISTGRGGNGTSSSHNWGNLFFASAFAFLGLLALTVTFSCNLATIKALVSRCRAKATASQSSAQWGRITTETAIQLMGIMCVLSVCWSPLLIMMLKMIFNQTSVEHCKTHTEKQKECNFFLIAVRLASLNQILDPWVYLLLRKILLRKFCQVANAVSSCSNDGQKGQPISLSNEIIQTEA

SEQ ID NO: 30 - DNA, 3261 bp, Homo sapiens
gcttggaagccagcgctgcgctccccgtgggaagcgatcgtctcctctgtcaactcgcgcctgggcacttagcccctcccgtttcagggcgccgcctccccggatggcaaacactataaagtggcggcgaataaggttcctcctgctgctctcggtttagtccaagatcagcgatatcacgcgtcccccggagcatcgcgtgcaggagccatggcgcgggagctataccacgaagagttcgcccgggcgggcaagcaggcggggctgcaggtctggaggattgagaagctggagctggtgcccgtgccccagagcgctcacggcgacttctacgtcggggatgcctacctggtgctgcacacggccaagacgagccgaggcttcacctaccacctgcacttctggctcggaaaggagtgttcccaggatgaaagcacagctgctgccatcttcactgttcagatggatgactatttgggtggcaagccagtgcagaatagagaacttcaaggatatgagtctaatgactttgttagctatttcaaaggcggtctgaaatacaaggctggaggcgtggcatctggattaaatcatgttcttacgaacgacctgacagccaagaggctcctacatgtgaagggtcgtagagtggtgagagccacagaagttccccttagctgggacagtttcaacaagggtgactgcttcatcattgaccttggcaccgaaatttatcagtggtgtggttcctcgtgcaacaaatatgaacgtctgaaggcaaaccaggtagctactggcattcggtacaatgaaaggaaaggaaggtctgaactaattgtcgtggaagaaggaagtgaaccctcagaacttataaaggtcttaggggaaaagccagagcttccagatggaggtgatgatgatgacattatagcagacataagtaacaggaaaatggctaaactatacatggtttcagatgcaagtggctccatgagagtgactgtggtggcagaagaaaaccccttctcaatggcaatgctgctgtctgaagaatgctttattttggaccacggggctgccaaacaaattttcgtatggaaaggtaaagatgctaatccccaagagaggaaggctgcaatgaagacagctgaagaatttctacagcaaatgaattattccaagaatacccaaattcaagttcttccagaaggaggtgaaacaccaatcttcaaacagttttttaaggactggagagataaagatcagagtgatggcttcgggaaagtttatgtcacagagaaagtggctcaaataaaacaaattccctttgatgcctcaaaattacacagttctccgcagatggcagcccagcacaatatggtggatgatggttctggcaaagtggagatttggcgtgtagaaaacaatggtaggatccaagttgaccaaaactcatatggtgaattctatggtggtgactgctacatcatactctacacctatcccagaggacagattatctacacgtggcaaggagcaaatgccacacgagatgagctgacaacatctgcgttcctgactgttcagttggatcggtcccttggaggacaggctgtgcagatccgagtctcccaaggcaaagagcctgttcacctactgagtttgttcaaagacaaaccgctcattatttacaagaatggaacatcaaagaaaggaggtcaggcacctgctccccctacacgcctctttcaagtccggagaaacctggcatctatcaccagaattgtggaggttgatgttgatgcaaattcactgaattctaacgatgtttttgtcctgaaactgccacaaaatagtggctacatctgggtaggaaaaggtgctagccaggaggaggagaaaggagcagagtatgtagcaagtgtcctaaagtgcaaaaccttaaggatccaagaaggcgaggagccagaggagttctggaattcccttggagggaaaaaagactaccagacctcaccactactggaaacccaggctgaagaccatccacctcggctttacggctgctctaacaaaactggaagatttgttattgaagagattccaggagagttcacccaggatgatttagctgaagatgatgtcatgttactagatgcttgggaacagatatttatttggattggcaaagatgctaatgaagttgagaaaaaagaatctctgaagtctgccaaaatgtaccttgagacagacccttctggaagagacaagaggacaccaattgtcatcataaaacagggccatgagccacccacattcacaggctggttcctgggctgggattccagcaagtggtaaattggtatttgtaaaaagcaaacaaacattacaaggcagttatctcattgctgttttgggagaggaacgggaaaagctttttgcttatttgtcttttgaaaattaaggctgggcgcggtggctcacacctgtaatcccagcactttgagaggatgaggtaggcggatcactggggtcaggatttcgagaccagcctggccaacatggcgaaacctcgcctctactaaaaatacaaaaaaattagctgcgcgtggtggtgcacgcctgtagtccctgctacttggaaggctgagacaggaaaattgcttgaacccaggaggctgaggttgcagtgagccaggattgcgccaccacactccagcctgggcaacagagactctgtctcaaaaaaaaaaaaaaaaaaaattaacctcaaccaaacgcctattttttaatgcttagttttggcttgaaattcttcttcacctggagttttcttacgttaatacattaaaataacctgaaggaaactttcgttatgggccaatattagctcttatggaaactgatttatatcttttttatgacctttcaaaagtaaaatactatgcataatctagaaagatttgtcagataggaaattttataatgcattagccattagtcagagttgttttttaacatgccagagaaaaagttgtaatgccttggaagttattctcttttctatagattgtgttcaaaagatgtgtatacttgcaataaggtttatgttaaggtggctttaacaattggttgctattttccttctttagtcaatatgactttgatgtcattaactgctaagtgttattttctaagaggaattttatcttttttttaaccttattaaaagcctttgaaaacacaaaaaaaaaaaaaaaa

SEQ ID NO: 31 - protein, 715 aa, Homo sapiens
MARELYHEEFARAGKQAGLQVWRIEKLELVPVPQSAHGDFYVGDAYLVLHTAKTSRGFTYHLHFWLGKECSQDESTAAAIFTVQMDDYLGGKPVQNRELQGYESNDFVSYFKGGLKYKAGGVASGLNHVLTNDLTAKRLLHVKGRRVVRATEVPLSWDSFNKGDCFIIDLGTEIYQWCGSSCNKYERLKANQVATGIRYNERKGRSELIVVEEGSEPSELIKVLGEKPELPDGGDDDDIIADISNRKMAKLYMVSDASGSMRVTVVAEENPFSMAMLLSEECFILDHGAAKQIFVWKGKDANPQERKAAMKTAEEFLQQMNYSKNTQIQVLPEGGETPIFKQFFKDWRDKDQSDGFGKVYVTEKVAQIKQIPFDASKLHSSPQMAAQHNMVDDGSGKVEIWRVENNGRIQVDQNSYGEFYGGDCYIILYTYPRGQIIYTWQGANATRDELTTSAFLTVQLDRSLGGQAVQIRVSQGKEPVHLLSLFKDKPLIIYKNGTSKKGGQAPAPPTRLFQVRRNLASITRIVEVDVDANSLNSNDVFVLKLPQNSGYIWVGKGASQEEEKGAEYVASVLKCKTLRIQEGEEPEEFWNSLGGKKDYQTSPLLETQAEDHPPRLYGCSNKTGRFVIEEIPGEFTQDDLAEDDVMLLDAWEQIFIWIGKDANEVEKKESLKSAKMYLETDPSGRDKRTPIVIIKQGHEPPTFTGWFLGWDSSKW

SEQ ID NO: 32 - DNA, 1284 bp, Homo sapiens
agttgttatacggcttttgtcttaacgaagccacccattacggtatgatgatgtcaaacgtgatgctgatgctacagttacagcccctgctggcgcagcctctctgattctctctccctctccgcgtccagtgctgggctttttcagacaagtgcatctcctaaccaggtcacatttcagccgcgacccactctccgccagtcaccggaggcagaccgcgggaggagagctgaggacagccgcgtgcgcttcgccagcagcggggtgggaggaaggacattaaaatactgcagaagtcaagacccccccaggtcgaacccagaccacgatgcgcgccccgggctgcgggcggctggtgctgccgctgctgctcctggccgcggcagccctggccgaaggcgacgccaaggggctcaaggagggcgagacccccggcaatttcatggaggacgagcaatggctgtcgtccatctcgcagtacagcggcaagatcaagcactggaaccgcttccgagacgttccctcgtccgacccacccagtacaacgcaggccacaccatgacctctcccgttttctccttaaactgcgtgcatccacaagttccttggaaatttgtttaaaacacacatgcttcataggctatccagccaagtgatgggaagaggcagagatttggatctttttgttaccgtcagacaagtgactgtttctccccaaagtcaaactggcaggttgtaattcacagaggacagcaggaaccacgggcagccaggtctccaggctgagtggccgggccccgcgatgggctgctgatctccgccttcgccccacatgcgacctgtccagccgcaggagcctggagccccgcgaggccttcccgaagcacctgcaggcccagcgcagatgagccgcagtctcctgccgcttcctgggcagcagcccggccacagcagcgagggggcggcagcaggccggcccatccggaatccatgtgtcactggcctaggcctccctccgactcgctggctgtttgatctgccaaaatgggaccagtgaaggtcagagggaaaagaaatgcttaatcagaacaggaaaggtttaaaataaaagattcctccccacctcccttcttccacctgcttccaatagccagctccgtgggtgagaataaatgacttactgcttgaccccatggcactgggctacccacctgtctctctgtattggtgccaaccacttcccccttcctctcatgctcatcagaagaaaggcactagatttga

SEQ ID NO: 33 - protein, 77 aa, Homo sapiens
MRAPGCGRLVLPLLLLAAAALAEGDAKGLKEGETPGNFMEDEQWLSSISQYSGKIKHWNRFRDVPSSDPPSTTQATP

SEQ ID NO: 34 - DNA, 4749 bp, Homo sapiens
acagagcgtgttgttagagctgtgctgagcgggtgtttgggttgttggctgctttcttccccctttctcacacacttgtatattattttgaggtggtgttcgcagagtttgaaaggagagagaattaaaaaaaaaagccgcaagcgtttcactcttttatttttataatccccttcaatttggggttaaaaaaaagacaagaaaacaggaaggaagagaaataaggaaatgagatgtggtaaaagaagctaaaaggtgccttttaaaagatcgttgctgtgaagtgaaaaaaatctccagagaaaccaaaaagcaccgccgagacctcttccgaaccaaaggagtttgtgtttgcttttagggaagaagaaagatcattcattcggaggaataacaaccaattaaaagacaaataaaaaaagtttggagtgggacgcagagcgagcgagaggagctgccggcgggcggtggggcgcggagcccgcactttcccggccgggtgagcggcggccgcggcgccgggctcggcgggtgcgcctcggcggagcgaacgtcggagcgttgccttgggagacgcgcgccggacaatgcccgcggcgggccagtgacgcccgcggggaatgcggagcggcccggcagccggcacccagccgccgccgcgcgttcctgccgcccgtgtcacgcgagacccggcgggggccgggaccgcccgagccgcccctcagaccgagccggccgcctccgctgccgcggccgcctcctcttcggggtcattaaagccaatgagccgcgcgcctctgccgagcgcagccaactaaatcggcttggatgattcgcgacctgagcaagatgtacccgcagaccagacacccggcaccgcatcagcctgctcaaccctttaaatttacaatttccgaatcctgtgatcggattaaggaagagtttcagtttttacaggctcaataccacagtctgaagctggaatgtgagaaactcgccagtgagaagacagagatgcagcggcattatgtcatgtattatgaaatgtcctatgggttgaatatagaaatgcacaagcaggcagagattgtcaagaggctgaatgctatctgtgcacaagtcattcctttcctgtcccaagagcaccagcaacaagtggtgcaggctgtggaacgggccaagcaggtgaccatggcagaactgaacgccatcattgggcaacaactccaggcccagcatttatcacatggacatggtctccccgtacctctgactccacacccttcagggctccagccccctgccattccacccatcggtagcagtgccgggcttctggccctctccagtgctctaggaggtcagtcccatcttccaattaaagatgagaagaagcaccatgacaatgatcaccaaagagacagagactccatcaagagctcttcagtatccccatcagccagtttccgaggtgctgagaagcacagaaactccgcagactactcctcagagagcaaaaagcagaaaactgaagaaaaggaaattgcagctcgttatgacagcgatggtgagaaaagtgatgacaacttggtggttgacgtttccaatgaggatccatcttcccctcgagggagcccagcacattcccccagagagaatggcctagacaagacacgcctgctcaagaaagatgccccgattagtccagcctctattgcatcttccagcagtactccctcctccaaatccaaagaacttagccttaatgaaaaatctactactcccgtctcaaagtccaatacccctactccacgaactgatgcgcccaccccaggcagtaactctactcccggattgaggcctgtacctggaaaaccaccaggagttgaccctttggcctcaagcctaaggaccccaatggcagtaccttgtccatatccaactccatttgggattgtgccccatgctggaatgaacggagagctgaccagccccggagcggcctacgctgggctccacaacatctcccctcagatgagcgcagctgctgccgccgccgctgctgctgctgcctatgggagatcaccagtggtgggatttgatccacaccatcacatgcgtgtgccagcaatacctccaaacctgacaggcattccaggaggaaaaccagcatactccttccatgttagcgcagatggtcagatgcagcctgtcccttttccacccgacgccctcatcggacctggaatcccccggcatgctcgccagatcaacaccctcaaccacggggaggtggtgtgcgcggtgaccatcagcaaccccacgagacacgtgtacacgggtgggaagggctgcgtcaaggtctgggacatcagccacccaggcaataagagtcctgtctcccagctcgactgtctgaacagggataactacatccgttcctgcagattgctccctgatggtcgcaccctaattgttggaggggaagccagtactttgtccatttgggacctggcggctccaaccccacgcatcaaggcagagctgacatcctcggcccccgcctgctatgccctggccatcagccccgattccaaggtctgcttctcatgctgcagcgacggcaacatcgctgtgtgggatctgcacaaccagaccttggtgaggcaattccagggccacacagatggagccagctgtattgacatttctaatgatggcaccaagctctggacaggtggtttggacaacacggtcaggtcctgggacctgcgcgaggggcggcagctgcagcagcacgacttcacctcccagatcttttctctgggctactgcccaactggagagtggcttgcagtggggatggagaacagcaatgtggaagttttgcatgtcaccaagccagacaaataccaactacatcttcatgagagctgtgtgctgtcgctcaagtttgcccattgtggcaaatggtttgtaagcactggaaaggacaaccttctgaatgcctggagaacaccttatggggccagtatattccagtccaaagaatcctcatcggtgcttagctgtgacatctccgtggacgacaaatacattgtcactggctctggggataagaaggccacagtttatgaagttatttattaaagacaaatcttcatgcagactggacttctcctcctggtagcactttgctctgtcatcctttttgttcacccccatccccgcatctaaaaccaaggatttcagatactcattgcagttgtggagtttaatcccctttcttaacctcacttcccacttgctattgaattgtgaatagtcattaaaaacctgtgataccaaatcttcagctgtctacttggaagaacatggaataagcatacttaacagtgaaaagaatctttaattatgtattatatctgtaatatatttattttgtttaaagaaggctttctaacaatgactgactaaataaagctgtctgctcctgcattgataatgaaggtgcgttgtatttgatacccctccccccctttttttggcaaaggaggggaaaggaaggtttaaaataattgatttaaaatgtcactaagtgtagactgatgactgtatagagatgtgaaatgtataattacacatggaagcaatatgttgctgtgttgttattaggttttttttgtttttgttttctacatcttttaaagacttttggaaatttggctgaacaattagaacacaacaggccaactcatactcatttggatctatttagacaacgttaaccaatatatctatagctttagattatattcgataaaagtaattggactttttttctttttttgactcgttgacaagtgtctttgtaatatgtttttagttccctttttttgttgtattataggcagatgaacaaattaaatttggcctcaaagagagaacttactcccttctggatatttttgccacatttctttgcaaaaggagatatatatatctttagtcagttttgttgttatgagaaattatgggttattttgtggcatgctctttgggagctgcacagttatggggaggactcccactgctgtgcaagttaagtcttttacaaaacaaggacagcagaggagggtttgcagagacctccctctgaaaaacacaaagaatggactctctcctgggatgaggacttgctttctttacctccggttctttccatgtcttagttggatgtccctgaaatggacacaggctgtgccattgtgccagaaacattgtgttatcttttatgttgttgttgttgctgttaaactataatatgtgacttctttttttattattttttgtttgaatgctttaaaaatcttttaagtctgtggatctgctgatgtacagtgcctttgctgctatggatcaaaatcaaaagaaccgtgtagatatactttattgtataagtagaaaattacttaatttcatactagaaatggatggatgctgcaagttgaaatggactgtccattgacgttcctaatgtggtagcagaaaaaaaaaaatggtgtcttaagtgcttagtgtttgatgtcattaacagtttcgtaaaactctacagtgtagaaagattttgatactaaactgtgcgttgtacatagttctaatgcattgtattgaccaccagtacttctataatggtagattgtttgtgaattcagacttttaagcattaaacataaataacttctagaa

SEQ ID NO: 35 - protein, 773 aa, Homo sapiens
MIRDLSKMYPQTRHPAPHQPAQPFKFTISESCDRIKEEFQFLQAQYHSLKLECEKLASEKTEMQRHYVMYYEMSYGLNIEMHKQAEIVKRLNAICAQVIPFLSQEHQQQVVQAVERAKQVTMAELNAIIGQQLQAQHLSHGHGLPVPLTPHPSGLQPPAIPPIGSSAGLLALSSALGGQSHLPIKDEKKHHDNDHQRDRDSIKSSSVSPSASFRGAEKHRNSADYSSESKKQKTEEKEIAARYDSDGEKSDDNLVVDVSNEDPSSPRGSPAHSPRENGLDKTRLLKKDAPISPASIASSSSTPSSKSKELSLNEKSTTPVSKSNTPTPRTDAPTPGSNSTPGLRPVPGKPPGVDPLASSLRTPMAVPCPYPTPFGIVPHAGMNGELTSPGAAYAGLHNISPQMSAAAAAAAAAAAYGRSPVVGFDPHHHMRVPAIPPNLTGIPGGKPAYSFHVSADGQMQPVPFPPDALIGPGIPRHARQINTLNHGEVVCAVTISNPTRHVYTGGKGCVKVWDISHPGNKSPVSQLDCLNRDNYIRSCRLLPDGRTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMENSNVEVLHVTKPDKYQLHLHESCVLSLKFAHCGKWFVSTGKDNLLNAWRTPYGASIFQSKESSSVLSCDISVDDKYIVTGSGDKKATVYEVIY

SEQ ID NO: 36 - DNA, 3752 bp, Homo sapiens
actcacgcctacgtactcagtcaccagcggaaccctctccaccaggctccgcgtcgaggccctacggggcgtcaatacgcacgcgtactttgtgtcccaggtagctcgtctcttagccatgtacgcctctgcattccggtcactgggagcggggccgtgggggctgcacatgcgcactgcgagtcctcgcctactcccagaaaccacccgggcgcatacgcggtgcggccgctgcttcaggtcggcgggctccagggagacctgggtgggcagcgtcgccgtttctcctttcttgggcagtatttttcccagcgccacgcggaggctgggccattatgagctctgcatttccaggacctggtcactattcaggacacggttccagcgcagtgcgcgttagccatgtctcagggatgagtgacattccaagatgtggccattgacttctccaaggaagagtggggattcctgaaccctgctcagagagatttgtacacaactgtgatgctggagaattatcagaacctggtctggctgggactttccatttctaaatctgtgatttcactgttggagaaaaggaaactgccttggataatggcaaaagaagagataagaggcccattgccaggatatttcaaggtgtcagagatgaccatctcccaggagccaaaggcaaagactagaactctctttggaaaaggttaaattatttactacacaatggctaagatttattttagcaaaagatataaaacaaaatcagcaaagatgtgccaggtgcagagattaaggagttatctgcaaagagggctattaatgaagtattatcgcagtttgacacagtgataaaatgtacaagaaacgtatgtaaggaatgtggaaatctatactgccacaatatgcagcttactctccataagagaaatcatacacaaaagaaatgcaatcagtgtttagattgtgggaaatacttcactcgtcaatcaactctcattcagcatcaaagaatccacacgggagagagaccctataaatgtaacgaatgtattaaaaccttcaaccagagggcacaccttacctagcatgagagaattcacactggtgagaaaccttacaaatgtaaggaatgcaggaaaaccttcagccagatgactcatctcacacagcatcagactacacatacgagagaaaagttccatgaatgcagtgaatgtggaaaggccttcagccgtgtctcagctcttatagatcaccagcgaattcatagtggagaaaagccgtatgaatgtaaggagtgtggaagagccttcactcaaagtgcccagctcattagacatcagaaaactcattctggagaaaaaccctatgagtgtagtaagtgtaagaaatcttttgtgcacctgtcttccctgattgaacattggagaattcacactggagaaaaaccatatcaatgtaaggactgcaaaaagaccttttgtcgtgtgatgcagttcactctgcacaggagaattcatactggtgaaaaaccctatgaatgcaaggaatgtggaaagtccttcagcgcccattcttctcttgttactcataagagaacacacagtggagaaaaaccgtataaatgcaaggaatgtggaaaagccttcagtgcgcactcttcccttgttactcataagagaacacacagtggagagaaaccctatacatgccatgcctgtgggaaggcctttaatacttcctccacactttgtcaacataatagaattcatactggtgaaaaaccctttcagtgcagtcaatgcgggaagtcctttagctgcagctctcaccttactcgacactgtagaatgtgtaatggaaaatttagcaaataaccaaaactcaaaatgttaattctgattattctgtatcatcaccagtactttgtactttgagtctttttaaatatgctctgttagtttgtatggaatgatatttcattgcacttttaatttaacttaaaaatgccttcccttgactgttgatatgtattttttgtgaagtgtgtgttgaagccttgcagattattttttaaattagaggtttatcactaggtatatctcctaatgctatccctcccccctccccctaatgtaaatgacgagttaatggtgcagcacaccaacatggcacatgtatacatatgtaacaaacctgcacgttgtgcacatggtaccctaaaacttaaagtataattaaaaaaaaaaattagaggtttttcttcgggtgcatttatgagagttcaccagattttggaaatattttctattctgtaacctaccttttcatttacataacagtgataaaagctttaagtttgatcacatccaattaatcacattttttaatagttatgcttttatttgtttttcagacagggtcttactctgtcacccaggctggagtgcagtggcacaatcatggctcactgcagtctctgcctcccaggctcaagtgatcctcccacctcagcctctcaagtagctgggactacaggcatgtgccaccacacctaatttctatattttttgtagagactaggtttcaccatgttgtccaggcttgtcttgacctcctgagctcaagcagtccatctgcctcagcctcccaaagtgctgggactacagacatgagccattgtgcctgacctagttctgctttttatgttctaagaagtattatttattctatgctgagcttacatagaatttcttccatgttttctatagctatagtgtatttttaccttttactgttagatctatttcatgtttttattatggataatggaggaggtaaaggttgaattatattttcccccatattattgacccatagtgacaccagttttgggagatgtctttcattttcgcccttacgttgttattggcaccttttgcaaaaatcaattatttatcatgagtatacttctgaaatgatctgtttcatcaatctatttgtcttctcagtagttccacattgtcttaattatagctttagaggaagtcttgaaatttggtagtaagtcttccagttttgttattttaacaaatttctctaggctgggcatggtggctcatgcctataatcccagcactttaggagccccagacaggaggatcacttaagcgcagaagatcaagaccagcctgggaaatatagcaagaccttatctctacaaaaaaatttagaaattagctgggcatggtggcacccatctgtagtcctaactgctcaggaggctgaggtgggaggattgcttgagtccaggagttcaaggctgcagtgagttgtgatcacaccattgcagtccagccaggttgacagaatgagaccctgtctcttaaaaaaaaaaaaaaaaatcctggtgatgcttttctggattaaaaaataaaataaaataaaaattggcctcactactctagcttatttgcattttctaataaatgttcagagtcaccttgtcagttcctaccaaaaagcctgtcggtattataattgagattgcatgaagtctgtagacaaacttgaagagaattgacacttaacagcagtgacttccagtctgtaaccttagcataagttctatttgtctcctttaatttttctcagaaatacctgtagttttctgtatagagatcttgaatgtattttcttaagtttattaatagacattttcacaatatgtaaaaaaaaaaaaaaaaaaaaaa

SEQ ID NO: 37 - protein, 146 aa, Homo sapiens
MLENYQNLVWLGLSISKSVISLLEKRKLPWIMAKEEIRGPLPDVPGAEIKELSAKRAINEVLSQFDTVIKCTRNVCKECGNLYCHNMQLTLHKRNHTQKKCNQCLDCGKYFTRQSTLIQHQRIHTGERPYKCNECIKTFNQRAHLT

SEQ ID NO: 38 - DNA, 3105 bp, Homo sapiens
caactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcaatgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttcccttgtagaaagtgaggctcagattaaattttagaatattttctaaatgtctttttcacaatcatgtactgggaaggcaatttcatactaaactgattaaataatacatttataatctacaactgtttgcacttacagctttttttgtaaatataaactataatttattgtctattttatatctgttttgctgtaacattgaaggaaagaccagacttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggca

SEQ ID NO: 39 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 40 - DNA, 627 bp, Homo sapiens
gcggccgcaagctcggcactcacggctctgagggctccgacggcactgacggccatggcgcgttcgaacctcccgctggcgctgggcctggccctggtcgcattctgcctcctggcgctgccacgcgacgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccagctggtggccggcatcaagtacttcctgacgatggagatggggagcacagactgccgcaagaccagggtcactggagaccacgtcgacctcaccacttgccccctggcagcaggggcgcagcaggagaagctgcgctgtgactttgaggtccttgtggttccctggcagaactcctctcagctcctaaagcacaactgtgtgcagatgtgataagtccccgagggcgaaggccattgggtttggggccatggtggagggcacttcaggtccgtgggccgtatctgtcacaataaatggccagtgctgcttcttgcaaaaaaaaaaaaaaaaaaa

SEQ ID NO: 41 - protein, 149 aa, Homo sapiens
MARSNLPLALGLALVAFCLLALPRDARARPQERMVGELRDLSPDDPQVQKAAQAAVASYNMGSNSIYYFRDTHIIKAQSQLVAGIKYFLTMEMGSTDCRKTRVTGDHVDLTTCPLAAGAQQEKLRCDFEVLVVPWQNSSQLLKHNCVQM

SEQ ID NO: 42 - DNA, 1216 bp, Homo sapiens
ggcagctgcacggctcctggccccggagcatgcgcgagagccgccccggagcgccccggagccccccgccgtcccgcccgcggcgtcccgcgccccgccgccagcgcacccccggacgctatggcccacccctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttt

SEQ ID NO: 43 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 44 - DNA, 39 bp, Artificial
  primer
aggaagagagtggtaaagaggaaaatgagtgataggatt

SEQ ID NO: 45 - DNA, 58 bp, Artificial
  primer
cagtaatacgactcactatagggagaaggctccacacacaaacctactaaaatccaaa

SEQ ID NO: 46 - DNA, 36 bp, Artificial
  primer
aggaagagaggtaayggggtaaattttatggtggtt

SEQ ID NO: 47 - DNA, 57 bp, Artificial
  primer
cagtaatacgactcactatagggagaaggctraactacactacaacctacrccctaa

SEQ ID NO: 48 - DNA, 40 bp, Artificial
  primer
aggaagagagttttttygtgaatygtttttgtattggtat

SEQ ID NO: 49 - DNA, 58 bp, Artificial
  primer
cagtaatacgactcactatagggagaaggctccrtcraaaccctcaaaaccrtaaata

SEQ ID NO: 50 - DNA, 40 bp, Artificial
  primer
aggaagagagtaggaattgagygaatttttttagtggttt

SEQ ID NO: 51 - DNA, 57 bp, Artificial
  primer
cagtaatacgactcactatagggagaaggctccaactccaccacaaacttacctaaa

SEQ ID NO: 52 - DNA, 38 bp, Artificial
  primer
aggaagagaggggagyggaatttttttttgtagtattt

SEQ ID NO: 53 - DNA, 57 bp, Artificial
  primer
cagtaatacgactcactatagggagaaggctaaaaacrcraaaaaactacccttaaa

SEQ ID NO: 54 - DNA, 40 bp, Artificial
  primer
aggaagagagagaggtataggttttggaaggtatagagtt

SEQ ID NO: 55 - DNA, 61 bp, Artificial
  primer
cagtaatacgactcactatagggagaaggcttcttcccraaactatataaactacaacaaa

SEQ ID NO: 56 - DNA, 40 bp, Artificial
  primer
aggaagagaggattttattagttttygygggtttygaatt

SEQ ID NO: 57 - DNA, 59 bp, Artificial
  primer
cagtaatacgactcactatagggagaaggctccraaactcccttccatcrttactataa

SEQ ID NO: 58 - DNA, 38 bp, Artificial
  primer
aggaagagaggtgaaatttgtttgtaaaagtgtggatt

SEQ ID NO: 59 - DNA, 60 bp, Artificial
  primer
cagtaatacgactcactatagggagaaggctaataaaaaccrctatcacatccatattaa

SEQ ID NO: 60 - DNA, 34 bp, Artificial
  primer
aggaagagaggttttttagatttagagaagttgg

SEQ ID NO: 61 - DNA, 58 bp, Artificial
  primer
cagtaatacgactcactatagggagaaggcttccraaacaaactatacaccttaacta

SEQ ID NO: 62 - DNA, 34 bp, Artificial
  primer
aggaagagagtyggagtagtttygtgattagatt

SEQ ID NO: 63 - DNA, 60 bp, Artificial
  primer
cagtaatacgactcactatagggagaaggctcctactaaaaccaaactaaacctactaaa

SEQ ID NO: 64 - DNA, 34 bp, Artificial
  primer
aggaagagagatagtgygatttagtaggttttgt

SEQ ID NO: 65 - DNA, 55 bp, Artificial
  primer
cagtaatacgactcactatagggagaaggctacctaaattcacaacaaacccata

SEQ ID NO: 66 - DNA, 40 bp, Artificial
  primer
aggaagagagtgaggttgttgattggttagtatagaagtt

SEQ ID NO: 67 - DNA, 56 bp, Artificial
  primer
cagtaatacgactcactatagggagaaggctacrcactcacctcacctcaatacta

SEQ ID NO: 68 - DNA, 40 bp, Artificial
  primer
aggaagagagygttaaattgttgttgtggtaatygtagtt

SEQ ID NO: 69 - DNA, 59 bp, Artificial
  primer
cagtaatacgactcactatagggagaaggcttatacccactctcraactaaacaacraa

SEQ ID NO: 70 - DNA, 39 bp, Artificial
  primer
aggaagagaggatattttagyggagaattggaggagatt

SEQ ID NO: 71 - DNA, 60 bp, Artificial
  primer
cagtaatacgactcactatagggagaaggctcractaaaactaaactaccccccataata

SEQ ID NO: 72 - DNA, 40 bp, Artificial
  primer
aggaagagagagaaattygaggttttaagtttaggtgttt

SEQ ID NO: 73 - DNA, 58 bp, Artificial
  primer
cagtaatacgactcactatagggagaaggctaaaaacrcttccttactacacccttaa

SEQ ID NO: 74 - DNA, 40 bp, Artificial
  primer
aggaagagagggtagtgatgggtatgtataaaggagtgtt

SEQ ID NO: 75 - DNA, 57 bp, Artificial
  primer
cagtaatacgactcactatagggagaaggctaccttattcrccrccactttataata

SEQ ID NO: 76 - DNA, 36 bp, Artificial
  primer
aggaagagagttgtgyggtttttggtgagaggtata

SEQ ID NO: 77 - DNA, 58 bp, Artificial
  primer
cagtaatacgactcactatagggagaaggctaacctccacctcaaaaattttaacaaa

SEQ ID NO: 78 - DNA, 37 bp, Artificial
  primer
aggaagagagagattaggygyggattttagtttggtt

SEQ ID NO: 79 - DNA, 59 bp, Artificial
  primer
cagtaatacgactcactatagggagaaggcttaaaacrctacraaaccraacaaatata

SEQ ID NO: 80 - DNA, 33 bp, Artificial
  primer
aggaagagaggygggaggagagttgaggatagt

SEQ ID NO: 81 - DNA, 55 bp, Artificial
  primer
cagtaatacgactcactatagggagaaggctaaccccraatctctaccttaacaa

SEQ ID NO: 82 - DNA, 40 bp, Artificial
  primer
aggaagagaggttatttgttagtagtgaaatgatgggtat

SEQ ID NO: 83 - DNA, 61 bp, Artificial
  primer
cagtaatacgactcactatagggagaaggctacacaactctaacaacacrctctatataaa

SEQ ID NO: 84 - DNA, 40 bp, Artificial
  primer
aggaagagaggaattygttaaatgtgygagttgggtagtt

SEQ ID NO: 85 - DNA, 57 bp, Artificial
  primer
cagtaatacgactcactatagggagaaggctaaataacaaccccaaacccaacaata

SEQ ID NO: 86 - DNA, 40 bp, Artificial
  primer
aggaagagagtttgtgttttaggtagttygttttttagtt

SEQ ID NO: 87 - DNA, 56 bp, Artificial
  primer
cagtaatacgactcactatagggagaaggctcctaaacraatccctaaacctctaa

SEQ ID NO: 88 - DNA, 20 bp, Artificial
  PRIMER
ttggagcaggaagaaacgca

SEQ ID NO: 89 - DNA, 20 bp, Artificial
  PRIMER
aacccgagcacaaaacaggc

SEQ ID NO: 90 - DNA, 22 bp, Artificial
  PRIMER
aagaggctgattggacggaaat

SEQ ID NO: 91 - DNA, 22 bp, Artificial
  PRIMER
tctacttgcactttgggcttgc

SEQ ID NO: 92 - DNA, 20 bp, Artificial
  PRIMER
aaggcaagacgctcagaggg

SEQ ID NO: 93 - DNA, 20 bp, Artificial
  PRIMER
gaggctccaaaatcacgctg

SEQ ID NO: 94 - DNA, 23 bp, Artificial
  PRIMER
aacctcagaccttccagatcgct

SEQ ID NO: 95 - DNA, 21 bp, Artificial
  PRIMER
agaggccgatttccttggtca

SEQ ID NO: 96 - DNA, 22 bp, Artificial
  PRIMER
cggtgataggatttttgggacc

SEQ ID NO: 97 - DNA, 22 bp, Artificial
  PRIMER
tgaaatccacagccacatcctc

SEQ ID NO: 98 - DNA, 22 bp, Artificial
  PRIMER
agggcttatgagtcccgatctc

SEQ ID NO: 99 - DNA, 20 bp, Artificial
  PRIMER
tcgactggtgcttgggtagg

SEQ ID NO: 100 - DNA, 22 bp, Artificial
  PRIMER
gcactttatagaccagcaccgg

SEQ ID NO: 101 - DNA, 21 bp, Artificial
  PRIMER
tggtactgctcatccgtcagg

SEQ ID NO: 102 - DNA, 22 bp, Artificial
  PRIMER
tcttacgaacgacctgacagcc

SEQ ID NO: 103 - DNA, 24 bp, Artificial
  PRIMER
acgaggaaccacaccactgataaa

SEQ ID NO: 104 - DNA, 20 bp, Artificial
  PRIMER
ccccgattagtccagcctct

SEQ ID NO: 105 - DNA, 23 bp, Artificial
  PRIMER
tggatatggacaaggtactgcca

SEQ ID NO: 106 - DNA, 22 bp, Artificial
  PRIMER
agtaagcaagaagagggcctgg

SEQ ID NO: 107 - DNA, 24 bp, Artificial
  PRIMER
accgcatctctttgggtactagaa

SEQ ID NO: 108 - DNA, 23 bp, Artificial Sequence
  PRIMER
gatgggagtggtaattggggttt

SEQ ID NO: 109 - DNA, 27 bp, Artificial Sequence
  PRIMER
atacaaccnaaccaaacnaaaactaaa

SEQ ID NO: 110 - DNA, 47 bp, Artificial Sequence
  PRIMER
tgtgtagatgtttttttaatatacaaccnaaccaaacnaaaactaaa

SEQ ID NO: 111 - DNA, 18 bp, Artificial Sequence
  PRIMER
aacgtctacacgcgtcga

SEQ ID NO: 112 - DNA, 28 bp, Artificial Sequence
  PRIMER
gggttgngggtttttttatttttagagt

SEQ ID NO: 113 - DNA, 49 bp, ARTIFICIAL
  PRIMER
acaccaaaaaaccaaaaccaagggttgngggtttttttatttttagagt

SEQ ID NO: 114 - DNA, 27 bp, ARTIFICIAL
  PRIMER
aacacaaaaaaaacaaaaaccccataa

SEQ ID NO: 115 - DNA, 38 bp, ARTIFICIAL
  PRIMER
tgggttttttgaacacaaaaaaaacaaaaaccccataa

SEQ ID NO: 116 - DNA, 14 bp, ARTIFICIAL
  PRIMER
aaccccgcgccaaa

SEQ ID NO: 117 - DNA, 36 bp, ARTIFICIAL
  PRIMER
tggaatgtggatcccttcncaatcactataatacaa

SEQ ID NO: 118 - DNA, 27 bp, ARTIFICIAL
  PRIMER
tanggggtngttattttgagattttag

SEQ ID NO: 119 - DNA, 25 bp, ARTIFICIAL
  PRIMER
tcccttcncaatcactataatacaa

SEQ ID NO: 120 - DNA, 16 bp, ARTIFICIAL
  PRIMER
tcaacgcctaacgaat

SEQ ID NO: 121 - DNA, 29 bp, ARTIFICIAL
  PRIMER
ttgtatttnggtgtagaagtttgggtagt

SEQ ID NO: 122 - DNA, 20 bp, ARTIFICIAL
  PRIMER
cncctaacncaccctcataa

SEQ ID NO: 123 - DNA, 39 bp, ARTIFICIAL
  PRIMER
tgttgttgggtgttgggtgcncctaacncaccctcataa

SEQ ID NO: 124 - DNA, 19 bp, ARTIFICIAL
  PRIMER
aataaacatctcccgcgcc

SEQ ID NO: 125 - DNA, 26 bp, ARTIFICIAL
  PRIMER
tttcgggttttatttcgtaggttagt

SEQ ID NO: 126 - DNA, 24 bp, ARTIFICIAL
  PRIMER
ctcccacccaaacctctaaattaa

SEQ ID NO: 127 - DNA, 39 bp, ARTIFICIAL
  PRIMER
tgatggtttggggttctcccacccaaacctctaaattaa

SEQ ID NO: 128 - DNA, 15 bp, ARTIFICIAL
  PRIMER
cgtcgaaaaacgcca

SEQ ID NO: 129 - DNA, 28 bp, ARTIFICIAL
  PRIMER
gagttttggngaggtagtttttatttgt

SEQ ID NO: 130 - DNA, 29 bp, ARTIFICIAL
  PRIMER
ccaaaaacnaacnaactaaaaaactaata

SEQ ID NO: 131 - DNA, 44 bp, ARTIFICIAL
  PRIMER
tggaggagggtgatgccaaaaacnaacnaactaaaaaactaata

SEQ ID NO: 132 - DNA, 19 bp, ARTIFICIAL
  PRIMER
catcttaactccgccccga